(19) United States
(12) Patent Application Publication
(10) Pub. No.: US 2025/0354163 A1
(43) Pub. Date: Nov. 20, 2025

(54) OPTIMIZED BASE EDITORS

(71) Applicants: BASF AGRICULTURAL SOLUTIONS US LLC, Research Traingle Park, NC (US); VIB VZW, Gent (BE); UNIVERSITEIT GENT, Gent (BE)

(72) Inventors: Frank MEULEWAETER, Gent Zwijnaarde (BE); David DE VLEESSCHAUWER, Gent Zwijnaarde (BE); Katelijn D'HALLUIN, Gent Zwijnaarde (BE); Thomas JACOBS, Gent (BE); Christophe GAILLOCHET, Gent (BE); Alexandra Pena FERNANDEZ, Gent (BE)

(73) Assignee: VB VZW, Gent (BE)

(21) Appl. No.: 18/853,012

(22) PCT Filed: Mar. 30, 2023
(86) PCT No.: PCT/EP2023/058232

§ 371 Date: Sep. 30, 2024

(30) Foreign Application Priority Data
Mar. 30, 2022 (EP) 22165461.9
Jan. 5, 2023 (EP) 23150383.0

(51) Int. Cl. C12N 15/82; C12N 9/22; C12N 9/78; C12N 15/11

(52) U.S. Cl. CPC C12N 15/8213; C12N 9/226; C12N 9/78; C12N 15/11; C12N 2310/20

(57) ABSTRACT

The present invention relates to an adenine base editor (ABE), and components thereof. The present invention also relates to a complex comprising an adenine base editor (ABE) and a guide RNA in a functionally associated form. The present invention further relates to a nucleic acid molecule encoding the ABE/guide RNA, an expression construct or a vector comprising a nucleic acid sequence encoding the adenine base editor and/or the nucleic acid sequence encoding the guide RNA. The present invention further relates to a cell comprising an adenine base editor (ABE) and a method of adenine base editing of a target site in a genome of interest in at least one cell of a prokaryotic organism, including bacterial and archaeal organisms, or eukaryotic organism. Besides that, the present invention relates to various methods, kits and uses associated with the ABEs provided.

### CROSS-REFERENCE TO RELATED APPLICATIONS

The present application is a national stage application filed under 35 U.S.C. 371 based on International Patent Application No. PCT/EP2023/058232, filed Mar. 30, 2023, which claims priority to EP Patent Application No. 22165461.9, filed Mar. 30, 2022, EP Patent Application No. 23150383.0, filed Jan. 5, 2023, the disclosures of each of which are hereby incorporated by reference in their entireties.

### REFERENCE TO SEQUENCE LISTING SUBMITTED ELECTRONICALLY

The sequence listing of the present application is submitted electronically as an XML file named “038771-00009_Substitute_SequenceListing.xml”, created on Jul. 2, 2025, and having a size of 374,120 bytes. This sequence listing submitted electronically is an integral part of the specification and is incorporated herein by reference in its entirety.

### TECHNICAL FIELD

The present invention relates to an adenine base editor (ABE), and components thereof. The present invention also relates to a complex comprising an adenine base editor (ABE) and a guide RNA in a functionally associated form. The present invention further relates to a nucleic acid molecule encoding the ABE/guide RNA, an expression construct or a vector comprising a nucleic acid sequence encoding the adenine base editor and/or the nucleic acid sequence encoding the guide RNA. The present invention further relates to a cell comprising an adenine base editor and a method of adenine base editing of a target site in a genome of interest in at least one cell of a prokaryotic organism, including bacterial and archaeal organisms, or eukaryotic organism. Besides that, the present invention relates to various methods, kits and uses associated with the ABEs provided.

### BACKGROUND OF THE INVENTION

Base editors meanwhile represent incredibly useful biotechnology tools that generate precise nucleotide substitutions at specific DNA target sites, particularly for site-specific eukaryotic and prokaryotic (including bacterial and archaeal) cell genome editing of complex genomes, where high precision is of utmost importance. There are currently two predominant types: cytidine/cytosine (CBE) and adenine/adenosine base editors (ABE). CBEs are usually created by fusing a cytidine deaminase domain to a catalytically-inactive Cas9, either the dead (D10A/H840A) or a nickase (D10A) Cas9. A variety of cytidine deaminases have been used for base editing including APOBEC1 (A1), A3A, A38, PmCDA1, AID, and their derivatives (Rees and Liu, 2018. Nat. Rev. Genet; doi: 10.1038/s41576-018-0059-1). CBEs catalyze the deamination of cytidines into uracil on the non-target DNA strand ultimately creating a C-G to T-A mutation (for CBEs, see Komor et al., Nature 533, 420-460, 2016; Komor et al., 2017, Science Advances, doi.10.1126/sciadv.aao4774). Regarding the Cas9 variant suitable for base editors, nCas9 is thought to be more active than dCas9 because nicking of the target strand causes the non-target strand to be used as a template in mismatch mediated repair (e.g., Eid et al., Biochem J. 2018 Jun. 15; 475(11): 1955-1964).

ABEs are derived from an evolved TadA from Escherichia coli(Gaudelli et al., Nature, 551: 464-471, 2017) and catalyze adenine into inosine, which is repaired as guanine, leading to A-T to G-C transitions. Similar to CBEs, the use of the D10A Cas9 nickase increases the frequency editing and editing windows are similar (Gaudelli et al., 2017, supra; Koblan et al., Nature Biotech, 36, 843-846, 2018). Unlike CBEs, the repair products are more accurate and fewer indels are observed (Rees and Liu, Nat Rev Genet, 19(12): 770-788, 2018).

CBEs and ABEs have now been used in a wide range of species and Cas9 has been the predominant nuclease platform, which has limited target range since the PAM and deamination window control the target space. Numerous groups have utilized alternative Cas9 PAM variants to overcome this limitation (e.g., Tan et al., Nat Comm, 110, 439, 2019).

Besides Cas9, Cas12a (earlier named Cpf1, a CRISPR class II Type V nuclease; see Zetsche et al., Cell, 163(3): 759-7741, 2015) represents another programmable DNA endonuclease guided by a single guide RNA (gRNA; sgRNA) that meanwhile represents an important tool for genome editing in higher eukaryotic cells, including plant cells (Bandyopadhyay et al., Front Plant Sci, 11: 58411, 2020). Meanwhile, various Cas12a variants with altered and enhanced PAM specificities were provided (Gao et al., Nat Biotechnol, 35: 789-792, 2017; Töth et al., Nucleic Acids Res., 48: 3722-3733, 2020). Furthermore, also temperature-tolerant variants of Cas12a have been described (Schindele and Puchta, 2020 Plant Biotechnology Journal, 18, 1118-1120). ABEs based on Cas9-variants in combination with TadA8e and TadA9 were also reported to have certain off-target effects in plants (Li et al., Genome Biology 2022, 23:51, https://doi.org/10.1186/s13059-022-02618-w) hampering a broad and targeted use of these ABEs.

Cas12a-derived base editors have also been reported occasionally, but systematic reports on the activity of base editors using Cas12a are not available at date and the functionality reported for Cas12a-derived editors in comparison to Cas9-based base editors is usually much lower in view of the fact that a highly active nickase variant of Cas12a (like the nCas9 D10A mutant) is not available. Particularly, a suitable Cas12a-derived base editor, let alone an ABE, suitable for applications in plants and having high activity and specificity is not yet available (cf. Molla et al., Nature Plants, 7: 1166-1187, 2021, see particularly p. 1173).

WO 20221020407A1 describes Cas12a-based ABEs, which show functionality in plants. These ABEs have a heterodimeric structure with regard to their adenosine deaminase domains, wherein one of the two adenosine deaminase domains is an evolved/mutated adenosine deaminase domain.

In view of the specific PAM targeting space of Cas12a in comparison to Cas9, Cas12a base editors would be of great interest for basic science as well as for precision genome editing applications in therapy, for applications in unicellular organisms (prokaryotic and eukaryotic, including yeast), and for plant genome editing. Cas12a nucleases cleave DNA in a distal region in relation to the respective PAM sequence, which is thus less critical for target binding and cleavage making Cas12a-based potentially very useful for gene inactivation, since the cleaved and subsequently repaired DNA sequence may be re-cleaved as the critical recognition motifs are maintained.

Usually, the optimization of genome editing tools such as base editors requires the laborious testing of large numbers of configurations (architectures) and targets (Komor et al., 2017, supra; Gaudelli et al., supra; Gao et al., supra), as generally applicable high-throughput platforms for testing and modifying new base editors are not yet available. Furthermore, findings for one base editor system, e.g. an nCas9 base CBE, cannot be simply extrapolated when trying to define a new base editor based on a different basic nuclease like Cas12a. As detailed above, CBEs and ABEs also significantly differ in structure, applicability and specificity.

Presently, optimized ABEs with a high average editing efficiency, a wide editing window, less off-target effects and having a broad applicability on various kinds of host cells are still missing. Meanwhile, the skilled person is aware of several TadA variants, orthologs and mutants identified in silico and evolved and tested for functionality in ABEs (cf. Zhang et al., Nature Communications, 2023, 14:414, https.//doi.org/10.1038/s41467-023-36003-3 and the Supplementary Data thereof). Still, exclusively focusing on specific Cas9-based ABEs and not even testing Cas12a-based ABEs or TadA9 alone, let alone in combination as ABE, Zhang et al. 2023 shows the significant difficulties associated with the identification of functional ABEs, as all functional moieties of an ABE fusion have to be optimized regarding the overall architecture and with respect to the individual moieties (protein and linker) to provide a functional ABE.

Even if certain ABEs may already be available for very specific purposes, the development of a novel ABE with improved functionality is extremely difficult. Developing, testing and validating such large fusion proteins requires starting from scratch, since, despite the large size of such fusion proteins, even the smallest changes may result in complete loss of function.

Using a systematic approach, called ITER (Iterative Testing of Editing Reagents) herein, it was thus an objective of the present invention to de novo develop and iteratively optimize a Cas12a-ABE by modifying various nuclear localization signal (NLS), linker, adenosine deaminase domain, and crRNA components in several iterative cycles to provide new ABE tools with applicability in broad range of target cells (prokaryotic and eukaryotic), showing high, targeted and specific activity for improving genome editing technologies in general, particularly in plants.

It was another object of the present invention to provide Cas12a-based ABEs, which are functional in plants and which have a comparatively simple domain architecture making them extremely versatile ad suitable for diverse applications. Cas-12a-based ABEs according to the present invention have a monomeric structure with regard to their adenosine deaminase domain. This simpler architecture in comparison to known Cas-12a-based ABEs, which are functional in plants, may allow for increased versatility in terms of use of the respective ABEs according to the present invention due to reduction of complexity. Furthermore, the comparatively simple architecture of the Cas12a-based results in a comparatively lower molecular weight and size, which is beneficial in terms of efficiency in many methods for gene delivery and transfection.

It was a further object of the present invention to provide Cas12a-based ABEs, which are functional in plants, wherein the comparatively simple domain architecture of the Cas12a-based ABEs still allows for an at least equal, preferably even better, editing efficiency as compared to known ABEs, which are functional in plants.

### BRIEF DESCRIPTION OF FIGURES

FIG. 1 a-b, Schematic representation of tested Cas12a base editor (BE) expression construct architectures (a) and guide RNA (gRNA) expression construct architectures (b). c, Fluorescent reporter system used for measuring ABE activity in wheat protoplasts. Upon AT to G:C base editing, mutation of a stop codon into a Gln (Q) codon restores a functional GFP coding sequence.

FIG. 2 a-c, Editing efficiencies measured by rate of GFP recovery (GFP cells/mCherry cells [%]) determined during iterative testing of different Cas12a base editor architectures in combination with different gRNA architectures. BE and gRNA architectures are referred to as numbers (1-12) and letters (a-h), respectively, as described in FIG. 1.

FIG. 3 a-b, Editing efficiencies in wheat protoplasts as measured by rate of GFP recovery (GFP cells/mCherry cells [%]) determined during iterative testing of different Cas12a base editor architectures in combination with different gRNA architectures. c, Key BE-gRNAs architectures obtained along the optimization path were compared side by side. Components leading to increased activity are shown at the right of the panel. BE and gRNA architectures are referred to as numbers (1-12) and letters (a-h), respectively, as described in FIG. 1.

FIG. 4 a, Schematic representation of tested Cas12a base editor (BE) protein architectures and guide RNA (gRNA) architectures. b, Editing efficiencies in maize protoplasts as measured by rate of GFP recovery (GFP cells/mCherry cells [%]) determined during iterative testing of different Cas12a base editor architectures in combination with different gRNA architectures. Components leading to increased activity are shown at the right of the panel.

FIG. 5 Base editing efficiencies of 6 Cas12a-ABE configurations in simplex as measured by the proportion of reads converted from AT to G:C in wheat. Different BE configurations are labelled with numbers and letters referring to FIGS. 1a and b. Barplot displays AT to G:C conversion rates at individual on-target target site. X-axis indicates targeted adenine at different positions along the protospacer, with the PAM-adjacent base being position 1. For each targeted adenine seven individual bar plots are shown, representing from left to right (i) a negative control (no BE), (ii) v6a, (iii) v9a, (iv) v6h, (v) v9h, (vi) v11h, (vii) v12h. Editing rates were calculated from 2 or 3 independent biological replicates that are depicted as dots on barplots. Violin plot represent pooled efficiencies at all target sites for individual base editor architecture. Significance is calculated with Kruskal-Wallis test with Dunn post-hoc test a P<0.05.

FIG. 6 Base editing efficiencies of 6 Cas12a-ABE configurations in multiplex as measured by the proportion of reads converted from AT to G:C in wheat. Different BE configurations are labelled with numbers and letters referring to FIGS. 1a and b. Barplot displays A:T to G:C conversion rates at individual on-target target site. X-axis indicates targeted adenine at different positions along the protospacer, with the PAM-adjacent base being position 1. For each targeted adenine seven individual bar plots are shown, representing from left to right (i) a negative control (no BE), (ii) v6a, (iii) v9a, (iv) v6h, (v) v9h, (vi) v11h, (vii) v12h. Editing rates were calculated from 3 independent biological replicates that are depicted as dots on barplots. Violin plot represent pooled efficiencies at all target sites for individual base editor architecture. Significance is calculated with Kruskal-Wallis test with Dunn post-hoc test a P<0.05.

FIG. 7 a-b, Frequency of T0 wheat plants with AT to G:C base editing at independent positions of the target site as measured by NGS (n>153) for each of the 2 base editors. Heterozygous (between 25% and 75% editing rate) and homozygous mutations (higher than 75%) are displayed. Two Cas12a-ABEs (v9h and v11h) are compared at TS60-A (a) and TS112-A (b). c-d, Frequency of individual T0 wheat plants carrying heterozygous or homozygous AT to G:C conversion at individual positions of the target site (n>153). Frequency for heterozygous (HZ) and homozygous (HM) T0 plants are shown in d) and combined frequencies in c). Values are depicted for TaTS60 and TaTS112 in the 3 subgenomes. Asterisks in (a-d) depicts significant difference in efficiency between v9h and v11h as measured by z-score test for two proportions (*: p<0.05; **: p<0.01; ***: p<0.001).

FIG. 8 a, Frequency of genotypes generated by Cas12a-ABE v9h and v11h. A>G indicates base editing measured at the target site position. Aa and aa denote heterozygous and homozygous base editing on subgenome A respectively. b, Percentage of T0 wheat plants carrying at least one mutation in one of the subgenome TS60 and/or TS112 loci. Asterisks depicts significant difference in editing efficiency between v9h and v11h as measured by z-score test for two proportions (*: p<0.05; **: p<0.01; ***: p<0.001).

FIG. 9 a-b, Comparison of editing rates as measured by ddPCR drop-off or NGS A:T to G:C conversion rate for TaTS60-A (a) or TaTS112-A (b) in individual T0 wheat plants (n>54) for each of 2 base editors (v9h and v11h). Gradient grey scale represents editing efficiency.

FIG. 10 Base editing efficiencies of 6 Cas12a-ABE configurations in multiplex as measured by the proportion of reads converted from AT to G:C in maize. Different BE configurations are labelled with numbers and letters referring to FIGS. 1a and b. Barplot displays AT to G:C conversion rates at individual on-target target site. X-axis indicates targeted adenine at different positions along the protospacer, with the PAM-adjacent base being position 1. For each targeted adenine seven individual bar plots are shown, representing from left to right (i) a negative control (no BE), (ii) v6a, (iii) v9a, (iv) v6h, (v) v9h, (vi) v11h, (vii) v12h. Editing rates were calculated from 3 independent biological replicates that are depicted as dots on barplots. Violin plot represent pooled efficiencies at all target sites for individual base editor architecture. Significance in (a-c) is calculated with Kruskal-Wallis test with Dunn post-hoc test a P<0.05.

FIG. 11 a-e shows: a-c the frequency of T0 maize plants with AT to G:C base editing at independent positions of target site Zm-TS3 (a), Zm-TS4 (b) and Zm-TS8 (c) as measured by Sanger sequencing for base editor v9h (n=25), v11h (n=27) and v12h (n=21). Heterozygous (between 25% and 75% editing rate) and homozygous mutations (higher than 75%) are displayed. d-e: the frequency of individual T0 maize plants carrying heterozygous or homozygous AT to G:C conversion at individual positions of target site Zm-TS1, Zm-TS3, Zm-TS4 and Zm-TS8. The activity of three base editors: v9h (n=25), v11h (n=27) and v12h (n=21) is compared. Frequency for heterozygous (HZ) and homozygous (HM) T0 plants are shown in e) and combined frequencies in d). Asterisks in (c-e) depict significant difference in efficiency between v12h and v9h or v11h as measured by z-score test for two proportions (*: p<0.05).

FIG. 12 shows the distribution of plants (wheat) with A-to-G edits in Cas12-ABE transgene free T1 generation. 12 independent T1 lines were analyzed. WT: wild type; HZ: heterozygous; HM; homozygous.

FIG. 13 a-b shows the ABE activity of TadA9>(GGGSS)6×>dLbCas12a-D156R in canola and soybean protoplasts. a. GFP fluorescence from of a defective GFP gene in canola protoplasts, reflecting the editing of a TAG stop codon into a functional CAG codon, compared to the fluorescence from a functional GFP gene (GFP control). b. Level of ABE activity at endogenous target sites as determined by NGS (in canola protoplasts) or ddPCR (in soybean protoplasts).

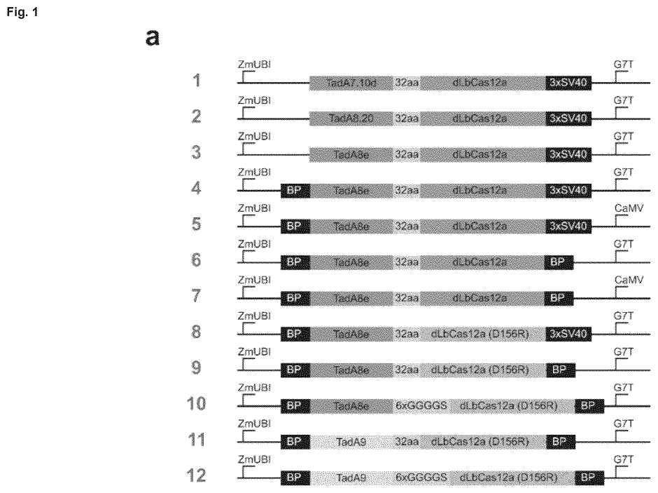

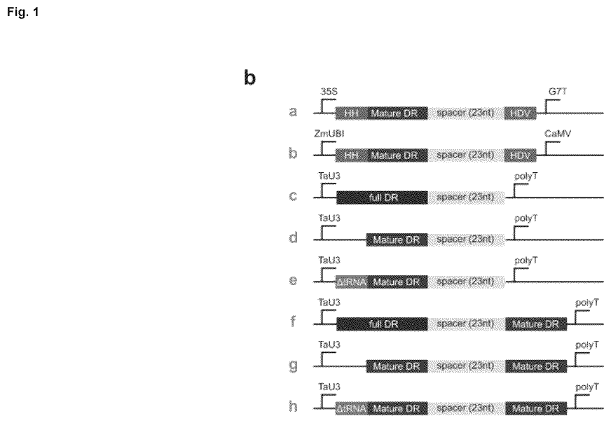

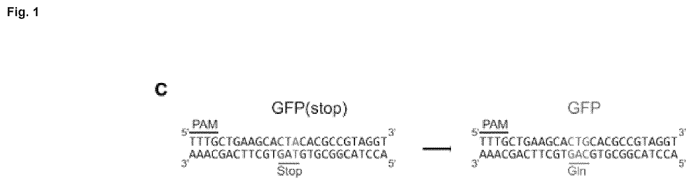

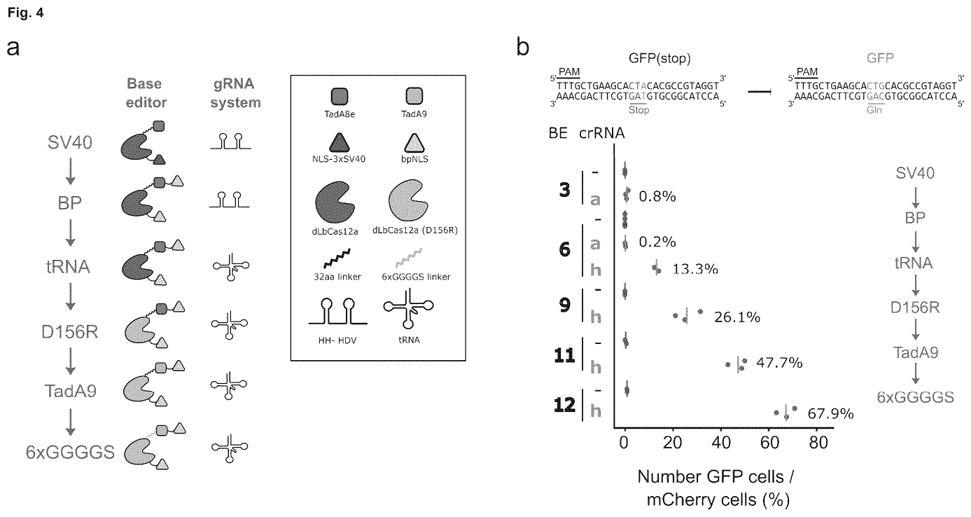

### BRIEF DESCRIPTION OF SEQUENCES

| SEQ ID | |
|---|---|
| NO. | Description |
| 1 | LbCas12a - Amino acid sequence of wildtype Cas12a protein from |
| | Lachnospiraceae bacterium |
| 2 | AsCas12a - Amino acid sequence of wildtype Cas12a protein from |
| | Acidaminococcus sp. (strain BV3L6) |
| 3 | FtCas12a - Amino acid sequence of wildtype Cas12a protein from |
| | Francisella tularensis (WP_216370596.1) |
| 4 | FnCas12a (FnCpf1) - Amino acid sequence of wildtype Cas12a protein |
| | from Francisella tularensis subsp. novicida |
| 5 | FnCas12a - Amino acid sequence of wildtype Cas12a protein from |
| | Francisella tularensis subsp. novicida (strain U112) |
| 6 | PcCas12a - Amino acid sequence of wildtype Cas12a protein from |
| | Porphyromonas crevioricanis |
| 7 | ErCas12a - Amino acid sequence of wildtype Cas12a protein from |
| | Eubacterium rectale |
| 8 | MmCas12a - Amino acid sequence of wildtype Cas12a protein from |
| | Methanomethylophilus alvus |
| 9 | MbCas12a - Amino acid sequence of wildtype Cas12a protein from |
| | Moraxella bovoculi |
| 10 | BsCas12a - Amino acid sequence of wildtype Cas12a protein from |
| | Butyrivibrio sp. NC3005 |
| 11 | TsCas12a - Amino acid sequence of wildtype Cas12a protein from |
| | Thiomicrospira sp. XS5 |
| 12 | Mb2Cas12a - Amino acid sequence of wildtype Cas12a protein from |
| | Moraxella bovoculi |
| 13 | Lb5Cas12a - Amino acid sequence of wildtype Cas12a protein from |
| | Lachnospiraceae bacterium |
| 14 | dLbCas12a - Amino acid sequence of DNase-dead mutant Cas12a |
| | protein from Lachnospiraceae bacterium (D832A mutant protein) |
| 15 | dLbCas12a - Amino acid sequence of DNase-dead mutant Cas12a |
| | protein from Lachnospiraceae bacterium (E925A mutant protein) |
| 16 | dLbCas12a - Amino acid sequence of DNase-dead mutant Cas12a |
| | protein from Lachnospiraceae bacterium (D832A/E925A mutant protein) |
| 17 | dAsCas12a - Amino acid sequence of DNase-dead mutant Cas12a |
| | protein from Acidaminococcus sp. (strain BV3L6; D908A mutant protein) |
| 18 | dAsCas12a - Amino acid sequence of DNase-dead mutant Cas12a |
| | protein from Acidaminococcus sp. (strain BV3L6; E993A mutant protein) |
| 19 | dAsCas12a - Amino acid sequence of DNase-dead mutant Cas12a |
| | protein from Acidaminococcus sp. (strain BV3L6;; D908A/E993A mutant |
| | protein) |
| 20 | dFtCas12a - Amino acid sequence of DNase-dead mutant Cas12a |
| | protein from Francisella tularensis (WP_216370596.1; D917A mutant |
| | protein) |
| 21 | dFtCas12a - Amino acid sequence of DNase-dead mutant Cas12a |
| | protein from Francisella tularensis (WP_216370596.1; E1006A mutant |
| | protein) |
| 22 | dFtCas12a - Amino acid sequence of DNase-dead mutant Cas12a |
| | protein from Francisella tularensis (WP_216370596.1; D917A/E1006A |
| | mutant protein) |
| 23 | dFnCas12a - Amino acid sequence of DNase-dead mutant Cas12a |
| | protein from Francisella tularensis subsp. novicida (D917A mutant protein) |
| 24 | dFnCas12a - Amino acid sequence of DNase-dead mutant Cas12a |
| | protein from Francisella tularensis subsp. novicida (E1006A mutant |
| | protein) |
| 25 | dFnCas12a - Amino acid sequence of DNase-dead mutant Cas12a |
| | protein from Francisella tularensis subsp. novicida (D917A/E1006A |
| | mutant protein) |
| 26 | dFnCas12a (strain U112) - Amino acid sequence of DNase-dead mutant |
| | Cas12a protein from Francisella tularensis subsp. novicida (strain U112; |
| | D917A mutant protein) |
| 27 | dFnCas12a (strain U112) - Amino acid sequence of DNase-dead mutant |
| | Cas12a protein from Francisella tularensis subsp. novicida (strain U112; |
| | E1006A mutant protein) |
| 28 | dFnCas12a (strain U112) - Amino acid sequence of DNase-dead mutant |
| | Cas12a protein from Francisella tularensis subsp. novicida (strain U112; |
| | D917A/E1006A mutant protein) |
| 29 | Amino acid sequence of DNase-dead, more active and/or thermotolerant |
| | mutant Cas12a protein from Lachnospiraceae bacterium (D832A/D156R |
| | mutant protein) |
| 30 | Amino acid sequence of DNase-dead, more active and/orthermotolerant |
| | mutant Cas12a protein from Lachnospiraceae bacterium (E925A/D156R |
| | mutant protein) |
| 31 | Amino acid sequence of DNase-dead, more active and/or thermotolerant |
| | mutant Cas12a protein from Lachnospiraceae bacterium (D832A/E925A/ |
| | D156R mutant protein) |
| 32 | Amino acid sequence of DNase-dead, more active and/or thermotolerant |
| | mutant Cas12a protein from Acidaminococcus sp. (strain BV3L6; D908A/ |
| | E174R mutant protein) |
| 33 | Amino acid sequence of DNase-dead, more active and/or thermotolerant |
| | mutant Cas12a protein from Acidaminococcus sp. (strain BV3L6; E993A/ |
| | E174R mutant protein) |
| 34 | Amino acid sequence of DNase-dead, more active and/or thermotolerant |
| | mutant Cas12a protein from Acidaminococcus sp. (strain BV3L6; D908A/ |
| | E993A/E174R mutant protein) |
| 35 | Amino acid sequence of DNase-dead, more active and/or thermotolerant |
| | mutant Cas12a protein from Francisella tularensis (WP_216370596.1; |
| | D917A/E184R mutant protein) |
| 36 | Amino acid sequence of DNase-dead, more active and/or thermotolerant |
| | mutant Cas12a protein from Francisella tularensis (WP_216370596.1; |
| | E1006A/E184R mutant protein) |
| 37 | Amino acid sequence of DNase-dead, more active and/or thermotolerant |
| | mutant Cas12a protein from Francisella tularensis (WP_216370596.1; |
| | D917A/E1006A/E184R mutant protein) |
| 38 | Amino acid sequence of DNase-dead, more active and/or thermotolerant |
| | mutant Cas12a protein from Francisella tularensis subsp. novicida (D917A/ |
| | E184R mutant protein) |
| 39 | Amino acid sequence of DNase-dead, more active and/or thermotolerant |
| | mutant Cas12a protein from Francisella tularensis subsp. novicida |
| | (E1006A/E184R mutant protein) |
| 40 | Amino acid sequence of DNase-dead, more active and/or thermotolerant |
| | mutant Cas12a protein from Francisella tularensis subsp. novicida (D917A/ |
| | E1006A/E184R mutant protein) |
| 41 | Amino acid sequence of DNase-dead, more active and/or thermotolerant |
| | mutant Cas12a protein from Francisella tularensis subsp. novicida (strain |
| | U112; D917A/E184R mutant protein) |
| 42 | Amino acid sequence of DNase-dead, more active and/or thermotolerant |
| | mutant Cas12a protein from Francisella tularensis subsp. novicida (strain |
| | U112; E1006A/E184R mutant protein) |
| 43 | Amino acid sequence of DNase-dead, more active and/or thermotolerant |
| | mutant Cas12a protein from Francisella tularensis subsp. novicida (strain |
| | U112; D917A/E1006A/E184R mutant protein) |
| 44 | Amino acid sequence of PAM recognition mutant of LbCas12a - RR |
| | mutant protein (G532R/K595R), recognizes TYCV PAM sites |
| 45 | Amino acid sequence of PAM recognition mutant of LbCas12a - RVR |
| | mutant protein (G532R/K538V/Y542R), recognizes TATV PAM sites |
| 46 | Amino acid sequence of PAM recognition mutant of LbCas12a - RVRR |
| | mutant protein (G532R/K538V/Y542R/K595R), recognizes TACV, CTCV, |
| | and CCCV PAM sites |
| 47 | Amino acid sequence of PAM recognition mutant of LbCas12a - |
| | enLbCas12a mutant protein (D156R/G532R/K538R), recognizes TTYN, |
| | VTTV, and TRTV PAM sites, plus the RVRR PAM sites |
| 48 | Amino acid sequence of XTEN 32aa linker |
| 49 | Amino acid sequence of XTEN 48aa linker |
| 50 | Amino acid sequence of GGGGS linker |
| 51 | Amino acid sequence of Hexa-GGGGS linker |
| 52 | Amino acid sequence of triple SV40 nuclear localization signal sequence |
| 53 | Amino acid sequence of bipartite SV40 nuclear localization signal |
| | sequence |
| 54 | Amino acid sequence of SV40 nuclear localization signal sequence |
| 55 | Amino acid sequence of flag-tagged SV40 nuclear localization signal |
| | sequence |
| 56 | Amino acid sequence of nucNLS, nuclear localization signal of the |
| | nucleoplasmin gene of Xenopus laevis |
| 57 | Amino acid sequence of transfer RNA adenine deaminase 8e sequence |
| | (TadA8e) |
| 58 | Amino acid sequence of transfer RNA adenine deaminase 9 sequence |
| | (TadA9) |
| 59 | DNA sequence for scaffold architecture for LbCas12a crRNA - Each |
| | scaffold architecture comprises in sequential order and in 5′ to 3′ |
| | orientation a truncated tRNA, one and/or more (mature) direct repeats, a |
| | spacer (target region) of variable length (18 to 30 nucleotides, preferably, |
| | 20 to 27 nucleotides, preferably 21 to 25 nucleotides, especially preferably |
| | 22 to 24 nucleotides), one and/or more (mature) direct repeats, and |
| | optionally a poly-T tail of variable length (3 to 10 nucleotides, preferably 5 |
| | to 9 nucleotides, especially preferably 6 to 8 nucleotides) |
| 60 | DNA sequence for scaffold architecture for AsCas12a crRNA - Each |
| | scaffold architecture comprises in sequential order and in 5′ to 3′ |
| | orientation a truncated tRNA, one and/or more (mature) direct repeats, a |
| | spacer (target region) of variable length (18 to 30 nucleotides, preferably, |
| | 20 to 27 nucleotides, preferably 21 to 25 nucleotides, especially preferably |
| | 22 to 24 nucleotides), one and/or more (mature) direct repeats, and |
| | optionally a poly-T tail of variable length (3 to 10 nucleotides, preferably 5 |
| | to 9 nucleotides, especially preferably 6 to 8 nucleotides) |
| 61 | DNA sequence for scaffold architecture for FnCas12a crRNA - Each |
| | scaffold architecture comprises in sequential order and in 5′ to 3′ |
| | orientation a truncated tRNA, one and/or more (mature) direct repeats, a |
| | spacer (target region) of variable length (18 to 30 nucleotides, preferably, |
| | 20 to 27 nucleotides, preferably 21 to 25 nucleotides, especially preferably |
| | 22 to 24 nucleotides), one and/or more (mature) direct repeats, and |
| | optionally a poly-T tail of variable length (3 to 10 nucleotides, preferably 5 |
| | to 9 nucleotides, especially preferably 6 to 8 nucleotides) |
| 62 | pCG392: pZmUBI-BP-TadA8e-XTEN linker-LbCas12a(D156R-D832A)- |
| | BP-G7T, 11321 bp - DNA sequence of complete construct for adenine |
| | base editor expression comprising in sequential order and in 5′ to 3′ |
| | orientation a promoter (pZmUBI), an NLS (Bipartite SV40), an adenine |
| | deaminase domain (TadA8e), a linker domain (XTEN linker), a dCas12a |
| | (dead, more active and/or thermotolerant D832A/D156R mutant of |
| | LbCas12a), an NLS (Bipartite SV40), and a G7 terminator |
| 63 | pCG434: pZmUBI-BP-TadA9-XTEN linker-LbCas12a(D156R-D832A)-BP- |
| | G7T, 11321 bp - DNA sequence of complete construct for adenine base |
| | editor expression comprising in sequential order and in 5′ to 3′ orientation |
| | a promoter (pZmUBI), an NLS (Bipartite SV40), an adenine deaminase |
| | domain (TadA9), a linker domain (XTEN linker), a dCas12a (dead, more |
| | active and/or thermotolerant D832A/D156R mutant of LbCas12a), an |
| | NLS (Bipartite SV40), and a G7 terminator |
| 64 | pCG463: pZmUBI-BP-TadA8e-6xGGGGS linker-LbCas12a(D156R- |
| | D832A)-BP-G7T, 11315 bp - DNA sequence of complete construct for |
| | adenine base editor expression comprising in sequential order and in 5′ to |
| | 3′ orientation a promoter (pZmUBI), an NLS (Bipartite SV40), an adenine |
| | deaminase domain (TadA8e), a linker domain (Hexa-GGGGS linker), a |
| | dCas12a (dead, more active and/or thermotolerant D832A/D156R |
| | mutant of LbCas12a), an NLS (Bipartite SV40), and a G7 terminator |
| 65 | pCG466: pZmUBI-BP-TadA9-6xGGGGS linker-LbCas12a(D156R- |
| | D832A)-BP-G7T, 11315 bp - DNA sequence of complete construct for |
| | adenine base editor expression comprising in sequential order and in 5′ to |
| | 3′ orientation a promoter (pZmUBI), an NLS (Bipartite SV40), an adenine |
| | deaminase domain (TadA9), a linker domain (Hexa-GGGGS linker), a |
| | dCas12a (dead, more active and/or thermotolerant D832A/D156R |
| | mutant of LbCas12a), an NLS (Bipartite SV40), and a G7 terminator |
| 66 | pTaU3-tRNA-matureDR- spacer-matureDR-polyT, 3349 bp - DNA |
| | sequence of complete construct for crRNA expression comprising in |
| | sequential order and in 5′ to 3′ orientation a promoter (TaU3), (truncated) |
| | tRNA, mature direct repeat, spacer (variable), mature direct repeat, poly-T |
| | tail |
| 67 | pCG406: pTaU3-tRNA-matureDR-TS60A spacer-matureDR-polyT, 3349 |
| | bp - DNA sequence of complete construct for crRNA expression |
| | comprising in sequential order and in 5′ to 3′ orientation a promoter |
| | (TaU3), (truncated) tRNA, mature direct repeat, spacer (TS60A spacer), |
| | mature direct repeat, poly-T tail |
| 68 | pCG408: pTaU3-tRNA-matureDR-TS112A gRNA-matureDR-polyT, 3349 |
| | bp - DNA sequence of complete construct for crRNA expression |
| | comprising in sequential order and in 5′ to 3′ orientation a promoter |
| | (TaU3), (truncated) tRNA, mature direct repeat, spacer (TS112A spacer), |
| | mature direct repeat, poly-T tail |
| 69 | Monocot pol III promoter pU3 wheat I |
| 70 | Monocot pol III promoter pU3 wheat II (cf. Marshallsay et al., 1992) |
| 71 | Monocot pol III promoter pU6.1 wheat (cf. Zhang et al., 2019; doi: |
| | 10.1111/pbi.13088) |
| 72 | Monocot pol III promoter pU6.3 wheat (cf. Zhang et al., 2019; doi: |
| | 10.1111/pbi.13088) |
| 73 | Monocot pol III promoter pU3 barley (cf. Kumar et al 2018; doi: |
| | 10.1111/pbi.12924) |
| 74 | Dicot pol III promoter Pu6 Arabidopsis thaliana (cf. Nekrasov et al., 2013) |
| 75 | Dicot pol III promoter P-u6-26 Arabidopsis thaliana from AT3G13855.1 (cf. |
| | Cai et al., 2018) |
| 76 | Dicot pol III promoter P-u6-29 Arabidopsis thaliana (cf. Ma et al., 2015; |
| | PubMed 25917172) |
| 77 | Dicot pol III promoter P-u6-6 Medicago truncatula (cf. Kim et al., 2013) |
| 78 | Dicot pol III promoter P-u3 Arabidopsis thaliana from AT5G54075.1 (cf. |
| | Ma et al., 2015) |
| 79 | Target site used to evaluate ABE activity at endogenous target in wheat |
| 80 | Target site used to evaluate ABE activity at endogenous target in wheat |
| 81 | Target site used to evaluate ABE activity at endogenous target in wheat |
| 82 | Target site used to evaluate ABE activity at endogenous target in wheat |
| 83 | Target site used to evaluate ABE activity at endogenous target in wheat |
| 84 | Target site used to evaluate ABE activity at endogenous target in wheat |
| 85 | Target site used to evaluate ABE activity at endogenous target in wheat |
| 86 | Target site used to evaluate ABE activity at endogenous target in wheat |
| 87 | Target site used to evaluate ABE activity at endogenous target in maize |
| 88 | Target site used to evaluate ABE activity at endogenous target in maize |
| 89 | Target site used to evaluate ABE activity at endogenous target in maize |
| 90 | Target site used to evaluate ABE activity at endogenous target in maize |
| 91 | Forward primer used for ddPCR drop-off assay to determine base editing |
| | levels in transgenic wheat plants at target site Ta-TS60-A |
| 92 | Reverse primer used for ddPCR drop-off assay to determine base editing |
| | levels in transgenic wheat plants at target site Ta-TS60-A |
| 93 | Reference probe (FAM) used for ddPCR drop-off assay to determine base |
| | editing levels in transgenic wheat plants at target site Ta-TS60-A |
| 94 | BE probe (HEX) used for ddPCR drop-off assay to determine base editing |
| | levels in transgenic wheat plants at target site Ta-TS60-A |
| 95 | Forward primer used for ddPCR drop-off assay to determine base editing |
| | levels in transgenic wheat plants at target site Ta-TS112-A |
| 96 | Reverse primer used for ddPCR drop-off assay to determine base editing |
| | levels in transgenic wheat plants at target site Ta-TS112-A |
| 97 | Reference probe (FAM) used for ddPCR drop-off assay to determine base |
| | editing levels in transgenic wheat plants at target site Ta-TS112-A |
| 98 | BE probe (HEX) used for ddPCR drop-off assay to determine base editing |
| | levels in transgenic wheat plants at target site Ta-TS112-A |
| 99 | FIG. 1C/FIG. 4B GFP Stop upper strand |
| 100 | FIG. 1C/FIG. 4B GFP Stop lower strand 5′ to 3′ |
| 101 | FIG. 1C/FIG. 4B GFP Gln upper strand |
| 102 | FIG. 1C/FIG. 4B GFP Gln lower strand 5′ to 3′ |
| 103 | pCG496_Maize-transformation_pZmUBI-BP-TadA8e- |
| | TaLbCas12a(D156R-D832A)-BP_Array-1-3-4-8 used in Example 11 |
| 104 | pCG497_Maize-transformation_pZmUBI-BP-TadA9-TaLbCas12a(D156R- |
| | D832A)-BP_Array-1-3-4-8 used in Example 11 |
| 105 | pCG498_Maize transformation_pZmUBI-BP-TadA9-6xGGGGS- |
| | TaLbCas12a(D156R-D832A)-BP_Array-1-3-4-8 used in Example 11 |
| 106 | Zm-TS1 forward primer |
| 107 | Zm-TS1 reverse primer |
| 108 | Zm-TS3 forward primer |
| 109 | Zm-TS3 reverse primer |
| 110 | Zm-TS4 forward primer |
| 111 | Zm-TS4 reverse primer |
| 112 | Zm-TS8 forward primer |
| 113 | Zm-TS8 reverse primer |
| 114 | Amino acid sequence of TadA-7.10 |
| 115 | Amino acid sequence of TadA8.20 |
| 116 | Amino acid sequence of alternative TadA8e (cf. SEQ ID NO: 57) with C- |
| | terminal extension |
| 117 | Amino acid sequence of alternative TadA9 (cf. SEQ ID NO: 58) with C- |
| | terminal extension |
| 118 | pBSU096: vector encoding a mutated GFP gene containing an early TAG |
| | stop codon instead of a CAG codon |
| 119 | pBSU215: pUbi10At > TadA9 > (GGGSS)6x > dLbCas12a-D156RCas12a- |
| | ABE > 3′pin2, 8720 bp - DNA sequence of complete adenine base editor |
| | expression construct comprising in sequential order and in 5′ to 3′ |
| | orientation a promoter (pUbi10At), an NLS (Bipartite SV40), an adenine |
| | deaminase domain (TadA9), a linker domain (Hexa-GGGGS linker), a |
| | dCas12a (dead, more active and/or thermotolerant D832A/D156R |
| | mutant of LbCas12a), an NLS (Bipartite SV40), and a terminator from the |
| | potato proteinase inhibitor II gene. |
| 120 | pBSU207: pAtU6-26 > matureDR-spacer-matureDR > polyT, 3228 bp - DNA |
| | sequence of complete construct for crRNA expression comprising in |
| | sequential order and in 5′ to 3′ orientation a promoter (AtU6-26), mature |
| | direct repeat, spacer targeting a defective GFP gene, mature direct repeat, |
| | poly-T |
| 121 | pBas05034: pAtU6-26 > matureDR-spacer-matureDR > polyT, 3228 bp - |
| | DNA sequence of complete construct for crRNA expression comprising in |
| | sequential order and in 5′ to 3′ orientation a promoter (AtU6-26), mature |
| | direct repeat, spacer targeting a FAD2 gene from Brassica napus, mature |
| | direct repeat, poly-T |
| 122 | pBSU343: pAtU6-26 > matureDR-spacer-matureDR > polyT, 3229 bp - DNA |
| | sequence of complete construct for crRNA expression comprising in |
| | sequential order and in 5′ to 3′ orientation a promoter (AtU6-26), mature |
| | direct repeat, spacer targeting an ALS3 gene from Brassica napus, mature |
| | direct repeat, poly-T |
| 123 | pBas04972: pGmU6 > matureDR-spacer-matureDR > polyT, 3106 bp - DNA |
| | sequence of complete construct for crRNA expression comprising in |
| | sequential order and in 5′ to 3′ orientation a promoter (GmU6), mature direct |
| | repeat, spacer targeting a FAD2 gene from Glycine max, mature direct |
| | repeat, poly-T |

“Identity” and/or “homology” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A AAGATACTG; length: 9 bases
Seq B: GATCTGA; length: 7 bases

Hence, the shorter sequence is sequence B.

Producing a pairwise global alignment which is showing both sequences over their complete lengths results in:

| | |
|---|---|
| Seq A: | AAGATACTG- |
| Seq B: | --GAT-CTGA |

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| Seq B: | GAT-CTGA |

Producing a pairwise alignment which is showing sequence A over its complete length according to the invention consequently results in:

| | |
|---|---|
| Seq A: | AAGATACTG |
| Seq B: | --GAT-CTG |

Producing a pairwise alignment which is showing sequence B over its complete length according to the invention consequently results in:

| | |
|---|---|
| Seq A: | GATACTG- |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

### DETAILED DESCRIPTION

In a first aspect, the present invention provides an adenine base editor (ABE), which may comprise, in sequential order, the following structural elements: a.) at least one N-terminal NLS sequence; b.) an adenosine deaminase domain being selected from a TadA9 domain and a TadA8 domain, preferably a TadA9 domain, or a functional variant of the aforementioned domains; c.) at least one linker domain; d.) a dCas12a, or a functional fragment thereof, or a nCas12a, or a functional fragment thereof, wherein the dCas12a or the nCas12a, or the a functional fragment thereof, comprises at least one or more mutations, wherein the at least one or more mutations confers increased activity and/or enhanced temperature tolerance, preferably wherein the one of the at least one or more mutations corresponds to a mutation in a dCas12a ortholog or homolog at a position homologous to D156 of SEQ ID NOs: 14, 15, or 16, E174 of SEQ ID NOs: 17, 18, or 19, and E184 of SEQ ID NOs: 20, 21, 22, 23, 24, 25, 26, 27, or 28, respectively, the at least one mutation conferring increased activity and/or enhanced temperature tolerance, particularly wherein the at least one mutation in the dCas12a ortholog or homolog corresponds to a D to R, an E to R, or a K to D/E mutation at the homologous position of any one of the deadCas12a variants of SEQ ID NOs: 14 to 43 as reference sequence, respectively; e.) at least one C-terminal NLS sequence; wherein the at least one N-terminal and the at least one C-terminal NLS sequence can be the same or different.

The skilled person is familiar with the nomenclature and structure of TadA molecules and the classification thereof (see Gaudelli et al., 2017 supra; Gaudelli et al., 2020; https://doi.org/10.1101/2020.03.13.990630). TadA8e, for example, is known to originate from TadA-7.10 (cf. SEQ ID NO: 114) by introducing 8 amino acid changes. TadA8.20 (cf. SEQ ID NO: 115) was also derived from TadA-7.10 but contained only 5 amino acid changes that are different from the ones in TadA8e. TadA9 was derived from TadA8e by introducing two of the amino acid mutations (V82S and Q154R) from TadA8.20, for example. As used herein, a certain class of TadA, e.g., TadA8e, TadA9, or TadA-7.10 means a molecule originating from TadA from Escherichia coli and having the characterizing mutations, also called signature mutations, of the respective TadA sub-class. Still, as the skilled person is aware of, certain further mutations, insertions or deletions at positions other than at the class-characterizing position may be present, e.g., a truncated N- or C-terminus, a mutation at a different site than at the class-characterizing position and the like. Such a variant having at least 80%, at least 85%, at least 90%, and preferably at least 95% sequence identity on an amino acid level to the corresponding TadA molecule will also be considered as falling under the same class. E.g., a TadA9 molecule having all the class-characterizing positions as the TadA9 sequence of SEQ ID NO: 58, but having certain variation (e.g., 4%) will still be considered as a TadA9 molecule as long as it has the overall deaminase functionality of the TadA9 and the class-characterizing positions as described in the art as shown with, for example, SEQ ID NO: 117. For example, a TadA8e (e.g., SEQ ID NO: 57, 116) or a TadA9 (e.g., SEQ ID NO: 58) have signature mutations at position 81 and 153, respectively, that allow the skilled person to identify the TadA class. Additionally, further mutations may be present that influence properties of the TadA other than the deaminase function. For example, in one embodiment a TadA, including TadA8e and TadA9, may comprise a mutation V105W at position 105 according to SEQ ID NO: 57 and 58 to reduce off-target activity and/or N107Q/S according to SEQ ID NO: 57 and 58 to further reduce cytosine deaminase activity (Jeong et al., 2021, Nature Biotechnology, https://doi.org/10.1038/s41587-021-00943-2). Further, in an additional embodiment, a TadA, including TadA8e and TadA9, may comprise a mutation F147A at position 147 according to SEQ ID NO: 57 and 58 to narrow the editing range (cf. Li et al., 2023, https.//doi.org/10.1016/jomtn.2022.12.001). With these additional mutation(s), a TadA8e and TadA9 will still be recognized as belonging to the TadA8e and TadA9 class, respectively, by one skilled in the art. Based on the above, a “functional variant” or a “functional fragment” in the context of a TadA or in the context of any dCas12, nCas12a or ABE as disclosed and claimed herein refers to a TadA, a dCas12a, an nCas12a or an ABE having the same class-characterizing (or signature) positions as the TadA it originates from, but a functional variant may be a shorter variant, for example, a truncated variant still comprising the relevant catalytically active site and the class-characterizing positions, or in another embodiment or aspect, for instance, a functional variant may be a molecule having high (>80%, preferably at least 90%, more preferably at least 95% on amino acid level) sequence identity to a TadA molecule it originates from and comprises certain mutations, but the variant still comprises the class-characterizing positions.

The terms “protein”, “polypeptide” and “amino acid sequence”, e.g. in the context of an adenine base editor, are used interchangeably herein.

The terms “adenine” and “adenosine”, e.g. in the context of a nucleic acid or a base editor, are used interchangeably herein.

The terms “cytosine” and “cytidine”, e.g. in the context of a nucleic acid or a base editor, are used interchangeably herein.

The term “in sequential order” as used herein in the context of a polypeptide/protein describes that the respective (sub-)element (also referred to as domain, moiety or (sub-) portion herein) is present in the overall polypeptide/protein in the specified sequential order from the N-terminus to the C-terminus of the amino acid sequence building up the polypeptide/protein. The term “in sequential order” also implies that any additional intervening sequence(s), linkers and the like can be present in between the moieties present in a given sequential order. When applied to “in sequential order” implies the orientation from the 5′ to the 3′ end of the respective nucleic acid sequence.

The term “structural element” as used herein, e.g. in the context of a protein or an adenine base editor, describes a region of a protein's polypeptide chain that represents a separate functional entity.

The term “NLS sequence” as used herein describes a nuclear localization signal, which is a part of a protein facilitating transport of the respective protein into the cell nucleus by means of nuclear transport. Typical characteristics of nuclear localization signals, such as the presence of positively charged amino acids like e.g. lysine and arginine are known to the skilled person. Mechanisms of nuclear transport are also known to the skilled person.

The term “increased activity and/or enhanced temperature tolerance” as used herein, i.e. in the context of adenine base editors (ABEs), describes an increase in enzymatic activity and/or an increase in temperature tolerance in active Cas12a, which may be induced by at least one or more mutations in the coding sequence of an active Cas12a, wherein the at least one or more mutations in the coding sequence lead to at least one or more amino acid exchanges in the amino acid sequence of the active Cas12a. In case an ABE comprises a Cas12, or a dCas12a, or a nCas12 carrying at least one or more mutations conferring increased activity and/or enhanced temperature tolerance as described above, an increased activity and/or enhanced temperature tolerance can thus, in turn, be conveyed to the ABE as such.

An adenine base editor according to the present invention may comprise at least one N-terminal NLS sequence, preferably one N-terminal NLS sequence, which is a Triple SV40 NLS sequence (3×SV40) corresponding to SEQ ID NO: 52 or having at least 90%, 91%, 92%, 93%, 94%, 95%, 96%, 97%, 98% or at least 99% sequence identity to SEQ ID NO: 52.

In one embodiment, an adenine base editor according to the present invention may comprise at least one N-terminal NLS sequence, preferably one N-terminal NLS sequence, which is a Bipartite SV40 NLS sequence (BP) corresponding to SEQ ID NO: 53 or having at least 90%, 91%, 92%, 93%, 94%, 95%, 96%, 97%, 98% or at least 99% sequence identity to SEQ ID NO: 53.

In one embodiment, an adenine base editor according to the present invention may comprise at least one N-terminal NLS sequence, preferably one N-terminal NLS sequence, which is an SV40 NLS sequence (SV40) corresponding to SEQ ID NO: 54 or having at least 90%, 91%, 92%, 93%, 94%, 95%, 96%, 97%, 98% or at least 99% sequence identity to SEQ ID NO: 54.

In one embodiment, an adenine base editor according to the present invention may comprise at least one N-terminal NLS sequence, preferably one N-terminal NLS sequence, which is a Flag-tagged SV40 nuclear localization signal sequence corresponding to SEQ ID NO: 55 or having at least 90%, 91%, 92%, 93%, 94%, 95%, 96%, 97%, 98% or at least 99% sequence identity to SEQ ID NO: 55.

In one embodiment, an adenine base editor according to the present invention may comprise at least one N-terminal NLS sequence, preferably one N-terminal NLS sequence, which is nucNLS, nuclear localization signal of the nucleoplasmin gene of Xenopus laevis corresponding to SEQ ID NO: 56 or having at least 90%, 91%, 92%, 93%, 94%, 95%, 96%, 97%, 98% or at least 99% sequence identity to SEQ ID NO: 56.

In yet another embodiment, an adenine base editor according to the present invention may comprise at least one or more N-terminal NLS sequence(s) selected from the group consisting of Triple SV40 NLS sequence (3×SV40) corresponding to SEQ ID NO: 52 or having at least 90%, 91%, 92%, 93%, 94%, 95%, 96%, 97%, 98% or at least 99% sequence identity to SEQ ID NO: 52 and Bipartite SV40 NLS sequence (BP) corresponding to SEQ ID NO: 53 or having at least 90%, 91%, 92%, 93%, 94%, 95%, 96%, 97%, 98% or at least 99% sequence identity to SEQ ID NO: 53 and SV40 NLS sequence (SV40) corresponding to SEQ ID NO: 54 or having at least 90%, 91%, 92%, 93%, 94%, 95%, 96%, 97%, 98% or at least 99% sequence identity to SEQ ID NO: 54 and Flag-tagged SV40 nuclear localization signal sequence corresponding to SEQ ID NO: 55 or having at least 90%, 91%, 92%, 93%, 94%, 95%, 96%, 97%, 98% or at least 99% sequence identity to SEQ ID NO: 55 and nucNLS, nuclear localization signal of the nucleoplasmin gene of Xenopus laevis corresponding to SEQ ID NO: 56 or having at least 90%, 91%, 92%, 93%, 94%, 95%, 96%, 97%, 98% or at least 99% sequence identity to SEQ ID NO: 56 or combinations thereof.

An adenine base editor according to the present invention may comprise at least one C-terminal NLS sequence, preferably one C-terminal NLS sequence, which is a Triple SV40 NLS sequence (3×SV40) corresponding to SEQ ID NO: 52 or having at least 90%, 91%, 92%, 93%, 94%, 95%, 96%, 97%, 98% or at least 99% sequence identity to SEQ ID NO: 52.

In one embodiment, an adenine base editor according to the present invention may comprise at least one C-terminal NLS sequence, preferably one C-terminal NLS sequence, which is a Bipartite SV40 NLS sequence (BP) corresponding to SEQ ID NO: 53 or having at least 90%, 91%, 92%, 93%, 94%, 95%, 96%, 97%, 98% or at least 99% sequence identity to SEQ ID NO: 53.

In one embodiment, an adenine base editor according to the present invention may comprise at least one C-terminal NLS sequence, preferably one C-terminal NLS sequence, which is an SV40 NLS sequence (SV40) corresponding to SEQ ID NO: 54 or having at least 90%, 91%, 92%, 93%, 94%, 95%, 96%, 97%, 98% or at least 99% sequence identity to SEQ ID NO: 54.

In one embodiment, an adenine base editor according to the present invention may comprise at least one C-terminal NLS sequence, preferably one C-terminal NLS sequence, which is a Flag-tagged SV40 nuclear localization signal sequence corresponding to SEQ ID NO: 55 or having at least 90%, 91%, 92%, 93%, 94%, 95%, 96%, 97%, 98% or at least 99% sequence identity to SEQ ID NO: 55.

In one embodiment, an adenine base editor according to the present invention may comprise at least one C-terminal NLS sequence, preferably one C-terminal NLS sequence, which is nucNLS, nuclear localization signal of the nucleoplasmin gene of Xenopus laevis corresponding to SEQ ID NO: 56 or having at least 90%, 91%, 92%, 93%, 94%, 95%, 96%, 97%, 98% or at least 99% sequence identity to SEQ ID NO: 56.

In one embodiment, an adenine base editor according to the present invention may comprise at least one or more C-terminal NLS sequence(s) selected from the group consisting of Triple SV40 NLS sequence (3×SV40) corresponding to SEQ ID NO: 52 or having at least 90%, 91%, 92%, 93%, 94%, 95%, 96%, 97%, 98% or at least 99% sequence identity to SEQ ID NO: 52 and Bipartite SV40 NLS sequence (BP) corresponding to SEQ ID NO: 53 or having at least 90%, 91%, 92%, 93%, 94%, 95%, 96%, 97%, 98% or at least 99% sequence identity to SEQ ID NO: 53 and SV40 NLS sequence (SV40) corresponding to SEQ ID NO: 54 or having at least 90%, 91%, 92%, 93%, 94%, 95%, 96%, 97%, 98% or at least 99% sequence identity to SEQ ID NO: 54 and Flag-tagged SV40 nuclear localization signal sequence corresponding to SEQ ID NO: 55 or having at least 90%, 91%, 92%, 93%, 94%, 95%, 96%, 97%, 98% or at least 99% sequence identity to SEQ ID NO: 55 and nucNLS, nuclear localization signal of the nucleoplasmin gene of Xenopus laevis corresponding to SEQ ID NO: 56 or having at least 90%, 91%, 92%, 93%, 94%, 95%, 96%, 97%, 98% or at least 99% sequence identity to SEQ ID NO: 56 or combinations thereof.

Particularly preferably, in certain embodiments, an adenine base editor according to the present invention may comprise one or more N-terminal and one or more C-terminal NLS sequence(s).

Especially preferably, an adenine base editor according to the present invention may comprise one or more N-terminal and one or more C-terminal NLS sequence(s), wherein the one or more N-terminal and C-terminal NLS sequence(s) is/are a Bipartite SV40 NLS sequence (BP) corresponding to SEQ ID NO: 53 or having at least 90%, 91%, 92%, 93%, 94%, 95%, 96%, 97%, 98% or at least 99% sequence identity to SEQ ID NO: 53.

The term “domain” as used herein describes a region of a protein's polypeptide chain that is self-stabilizing and that preferably folds independently from the rest of the protein.

The term “adenosine deaminase domain” as used herein describes a part of a protein and/or fusion protein facilitating the deamination of adenosine to inosine by substitution of an amino group by a keto group catalysed by the respective protein and/or fusion protein.

Suitable adenosine deaminase domains are disclosed herein, or are known to the skilled person (Huang et al., 2021, Nature Protocols, 16, 1089-1128; doi: 10.1038/s41596-020-00450-9).

The adenine base editor according to the present invention may comprise an adenosine deaminase domain, which is a TadA8 domain, preferably a TadA8e domain corresponding to SEQ ID NO: 57 or corresponding to a sequence having at least 85%, 86%, 87%, 88%, 89%, 90%, 91%, 92%, 93%, 94%, 95%, 96%, 97%, 98% or at least 99% sequence identity to SEQ ID NO: 57.

In one embodiment, the adenine base editor according to the present invention may comprise an adenosine deaminase domain, which is a TadA9 domain, preferably a TadA9 domain corresponding to SEQ ID NO: 58 or corresponding to a sequence having at least 85%, 86%, 87%, 88%, 89%, 90%, 91%, 92%, 93%, 94%, 95%, 96%, 97%, 98% or at least 99% sequence identity to SEQ ID NO: 58.

The term “linker domain” as used herein describes a part of a fusion protein connecting two functional domains of the respective fusion protein and thus facilitating the prevention of undesired effects, such as misfolding of the respective fusion protein. Particularly, the linker can guarantee a proper spacing between different elements so that each structural element or entity may exert its function within the fusion correctly.

The adenine base editor according to the present invention may comprise at least one linker domain, preferably an XTEN 32aa linker domain, especially preferably an XTEN 32aa linker domain corresponding to SEQ ID NO: 48 or corresponding to a sequence having at least 90%, 91%, 92%, 93%, 94%, 95%, 96%, 97%, 98% or at least 99% sequence identity to any of the sequences corresponding to SEQ ID NO: 48.

In one embodiment, the adenine base editor according to the present invention may comprise at least one linker domain, preferably an XTEN 48aa linker domain, especially preferably an XTEN 48aa linker domain corresponding to SEQ ID NO: 49 or corresponding to a sequence having at least 90%, 91%, 92%, 93%, 94%, 95%, 96%, 97%, 98% or at least 99% sequence identity to any of the sequences corresponding to SEQ ID NO: 49.

In one embodiment, the adenine base editor according to the present invention may comprise one or more linker domain(s) selected from the group consisting of sequences corresponding to SEQ ID NO: 48, SEQ ID NO: 49, SEQ ID NO: 50, SEQ ID NO: 51, or one or more linker domain(s) individually corresponding to a sequence having at least 90%, 91%, 92%, 93%, 94%, 95%, 96%, 97%, 98% or at least 99% sequence identity to any of the sequences corresponding to SEQ ID NO: 48, 49, 50, or 51.

In one embodiment, the adenine base editor according to the present invention may comprise at least one linker domain, preferably a GGGGS linker domain corresponding to SEQ ID NO: 50.

Particularly preferably, the adenine base editor according to the present invention may comprise at least one linker domain, preferably a Hexa-GGGGS linker domain corresponding to SEQ ID NO: 51.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a XTEN 32aa linker corresponding to SEQ ID NO: 48, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a XTEN 32aa linker corresponding to SEQ ID NO: 48, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 32aa linker corresponding to SEQ ID NO: 48, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 32aa linker corresponding to SEQ ID NO: 48, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 32aa linker corresponding to SEQ ID NO: 48, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a Hexa-GGGGS linker corresponding to SEQ ID NO: 51, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a XTEN 32aa linker corresponding to SEQ ID NO: 48, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 32aa linker corresponding to SEQ ID NO: 48, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a Hexa-GGGGS linker corresponding to SEQ ID NO: 51, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a XTEN 32aa linker corresponding to SEQ ID NO: 48, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 32aa linker corresponding to SEQ ID NO: 48, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 32aa linker corresponding to SEQ ID NO: 48, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 523, a TadA8e domain corresponding to SEQ ID NO: 57, an XTEN 32aa linker corresponding to SEQ ID NO: 48, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a XTEN 48aa linker corresponding to SEQ ID NO: 49, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a XTEN 48aa linker corresponding to SEQ ID NO: 49, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 29, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 30, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 31, and a bipartite SV40 nuclear localization signal corresponding to SEQ ID NO: 53.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a GGGGS linker corresponding to SEQ ID NO: 50, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, a XTEN 48aa linker corresponding to SEQ ID NO: 49, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA9 domain corresponding to SEQ ID NO: 58, an XTEN 48aa linker corresponding to SEQ ID NO: 49, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a GGGGS linker corresponding to SEQ ID NO: 50, a dCas12a according to SEQ ID NO: 31, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, a XTEN 48aa linker corresponding to SEQ ID NO: 49, a dCas12a according to any one of SEQ ID NO: 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 29, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 30, and a triple SV40 nuclear localization signal corresponding to SEQ ID NO: 52.

In one embodiment, the adenine base editor according to the present invention may comprise, in sequential order, a bipartite SV40 nuclear localization signal corresponding to SEQ ID NO: 53, a TadA8e domain corresponding to SEQ ID NO: 57, an XTEN 48aa linker corresponding to SEQ ID NO: 49, a dCas12a according to SEQ ID NO: 31, and a triple SV40 nuclear localization signal corresponding to SEQ ID NO: 52.

The term “dCas12a” as used herein describes any mutant of an ortholog of Cas12a harbouring at least one mutation significantly diminishing or abolishing at least the DNase activity of the corresponding wildtype Cas12a enzyme. Such DNase-dead mutants of a Cas12a nuclease (dead Cas12a), or functional fragments thereof, may comprise one, two, three, or more mutations, especially preferably one or two mutations, rendering their respective DNase activity at least diminished or even abolished. Preferably, the one, two, three, or more mutations, especially preferably one or two mutations, rendering the respective nuclease activity non-functional are located in the nuclease active site, e.g. the RuvC site, of the respective Cas12a nuclease, or functional fragment thereof.

The term “functional fragment” as used herein defines a sub-domain of an enzyme or protein used, particularly of a Cas12a or a TadA, that is able to fold and to exert at least one function of the full-length protein it is derived from, but which only comprises at least one functional domain or fragment of the full-length protein. The functional fragment may also include an N-terminally or C-terminally truncated version of the corresponding full-length protein. In any case, a functional fragment will be smaller and thus sterically less demanding than the corresponding full-length protein. In the context of an ABE, as such representing a multi-domain protein, the term “functional fragment” or “functional variant” refers to an ABE with substantially the same overall architecture regarding the CRISPR effector and the TadA deaminase and the position of at least one linker, but comprising certain additional mutations or domains, which additional mutations or domains, however, do not influence the overall ABE base editor activity as measurable by monitoring a given ABE yielding editing activity at at least one target site of interest.

The term “genome” as used herein describes all genetic information of an organism, which consists of nucleotide sequences, which may exist in the form of deoxyribonucleic acid (DNA) and/or ribonucleic acid (RNA).

The term “target site” as used herein describes a nucleotide sequence, typically a DNA sequence, which can be subjected to base editing using a base editor as described herein. Typically, a target site is part of a genome.

The term “PAM” as used herein describes a protospacer adjacent motif, which is a short nucleotide sequence, typically a DNA sequence, which typically is about 2 to 6 base pairs long and which is located within or in proximity to a given target site. Different types of nucleases recognize and bind to one or more specific PAM sequence(s). In case of Cas9 nucleases, Cas-9-mediated DNA cleavage occurs in the PAM-proximal sequence region. In case of Cas12a nucleases, Cas12a-mediated DNA cleavage occurs in a more distal region in relation to the respective PAM sequence.

The terms “PAM” and “PAM sequence” are used interchangeably herein.

Preferably, the adenine base editor according to the present invention may comprise a dCas12a, or functional fragment thereof, wherein the RNA processing activity of the dCas12a, or functional fragment thereof, is not affected by the at least one mutation significantly diminishing or abolishing at least the DNase activity of the corresponding wildtype Cas12a enzyme.

The adenine base editor according to the present invention may comprise a dCas12a, or a functional fragment thereof, wherein one of the one, two, three, or more mutations, especially preferably one or two mutations, rendering the nuclease activity non-functional correspond(s) to a mutation in a Cas12a ortholog or homolog, or a functional fragment thereof, at a position homologous to D832 of SEQ ID NOs: 1, 13, 15, 30, 44, 45, 46, or 47, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 15, 30, 44, 45, 46, or 47, or a functional fragment thereof.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to a mutation in a Cas12a ortholog or homolog, or a functional fragment thereof, at a position homologous to D908 of SEQ ID NOs: 2, 18, or 33, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 2, 18, or 33, or a functional fragment thereof.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to a mutation in a Cas12a ortholog or homolog, or a functional fragment thereof, at a position homologous to D917 of SEQ ID NOs: 3, 4, 5, 21, 24, 27, 36, 39, or 42, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 3, 4, 5, 21, 24, 27, 36, 39, or 42, or a functional fragment thereof.

Preferably, the adenine base editor according to the present invention may comprise a dCas12a, or a functional fragment thereof, wherein one of the one, two, three, or more mutations, especially preferably one or two mutations, rendering the nuclease activity non-functional corresponds to a D to A mutation in a Cas12a ortholog or homolog.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to a mutation in a Cas12a ortholog or homolog, or a functional fragment thereof, at a position homologous to E925 of SEQ ID NOs: 1, 13, 14, 29, 44, 45, 46, or 47, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 29, 44, 45, 46, or 47, or a functional fragment thereof.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to a mutation in a Cas12a ortholog or homolog, or a functional fragment thereof, at a position homologous to E993 of SEQ ID NOs: 2, 17, or 32, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 2, 17, or 32, or a functional fragment thereof.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to a mutation in a Cas12a ortholog or homolog, or a functional fragment thereof, at a position homologous to E1006 of SEQ ID NOs: 3, 4, 5, 20, 23, 26, 35, 38, or 41, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 3, 4, 5, 20, 23, 26, 35, 38, or 41, or a functional fragment thereof.

In one embodiment, the adenine base editor according to the present invention may comprise a dCas12a, or a functional fragment thereof, wherein one of the one, two, three, or more mutations, especially preferably one or two mutations, rendering the nuclease activity non-functional corresponds to an E to A mutation in a Cas12a ortholog or homolog.

In another embodiment, the adenine base editor according to the present invention may comprise a dCas12a, or a functional fragment thereof, wherein the dCas12a, or the functional fragment thereof, comprises at least one or more mutations conferring increased activity and/or enhanced temperature tolerance.

In one embodiment, the adenine base editor according to the present invention may comprise a dCas12a, or a functional fragment thereof, wherein the dCas12a, or the functional fragment thereof, comprises at least one or more mutations that confer increased activity and/or enhanced temperature tolerance, if present in a Cas12a.

In one embodiment, the adenine base editor according to the present invention may comprise a dCas12a, or a functional fragment thereof, wherein one of the at least one or more mutations conferring enhanced temperature tolerance corresponds to a mutation in a dCas12a ortholog or homolog, or a functional fragment thereof, at a position homologous to D156 of SEQ ID NOs: 1, 13, 14, 15, 44, 45, 46, or 47, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 44, 45, 46, or 47.

In one embodiment, the adenine base editor according to the present invention may comprise a dCas12a, or a functional fragment thereof, wherein one of the at least one or more mutations conferring enhanced temperature tolerance corresponds to a mutation in a dCas12a ortholog or homolog, or a functional fragment thereof, at a position homologous to E174 of SEQ ID NOs: 2, 17, or 18, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 2, 17, or 18.

In one embodiment, the adenine base editor according to the present invention may comprise a dCas12a, or a functional fragment thereof, wherein one of the at least one or more mutations conferring enhanced temperature tolerance corresponds to a mutation in a dCas12a ortholog or homolog, or a functional fragment thereof, at a position homologous to E184 of SEQ ID NOs: 3, 4, 5, 20, 21, 23, 24, 26, or 27, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 3, 4, 5, 20, 21, 23, 24, 26, or 27.

In one embodiment, the adenine base editor according to the present invention may comprise a dCas12a, or a functional fragment thereof, wherein one of the at least one or more mutations conferring increased activity and/or enhanced temperature tolerance corresponds to a D to R mutation.

In one embodiment, the adenine base editor according to the present invention may comprise an nCas12a, or a functional fragment thereof, wherein the nCas12a, or the functional fragment thereof, comprises at least one or more mutations that confer increased activity and/or enhanced temperature tolerance, if present in a Cas12a.

In one embodiment, the adenine base editor according to the present invention may comprise a dCas12a, or a functional fragment thereof, or a nCas12a, or a functional fragment thereof, wherein the dCas12a or the nCas12a, or a functional fragment thereof, comprises at least one or more mutations, wherein the at least one or more mutations confer increased activity and/or enhanced temperature tolerance, and wherein one of the least one or more mutations corresponds to a mutation in a dCas12a ortholog or homolog at a position homologous to D156 of SEQ ID NOs: 14, 15, or 16, and wherein the at least one mutation in the dCas12a ortholog or homolog corresponds to a D to R mutation at the homologous position.

In another embodiment, the adenine base editor according to the present invention may comprise a dCas12a, or a functional fragment thereof, wherein one of the at least one or more mutations conferring increased activity and/or enhanced temperature tolerance is an E to R mutation. In one embodiment, the adenine base editor according to the present invention may comprise a dCas12a, or a functional fragment thereof, or a nCas12a, or a functional fragment thereof, wherein the dCas12a or the nCas12a, or a functional fragment thereof, comprises at least one or more mutations, wherein the at least one or more mutations confer increased activity and/or enhanced temperature tolerance, and wherein one of the least one or more mutations corresponds to a mutation in a dCas12a ortholog or homolog at a position homologous to E174 of SEQ ID NOs: 17, 18, or 19, and wherein the at least one mutation in the dCas12a ortholog or homolog corresponds to a E to R mutation at the homologous position.

In one embodiment, the adenine base editor according to the present invention may comprise a dCas12a, or a functional fragment thereof, or a nCas12a, or a functional fragment thereof, wherein the dCas12a or the nCas12a, or a functional fragment thereof, comprises at least one or more mutations, wherein the at least one or more mutations confer increased activity and/or enhanced temperature tolerance, and wherein one of the least one or more mutations corresponds to a mutation in a dCas12a ortholog or homolog at a position homologous to E184 of SEQ ID NOs: 20, 21, 22, 23, 24, 25, 26, 27, or 28, and wherein the at least one mutation in the dCas12a ortholog or homolog corresponds to a E to R mutation at the homologous position.

In yet another embodiment, the adenine base editor according to the present invention may comprise a dCas12a, or a functional fragment thereof, wherein one of the at least one or more mutations conferring enhanced temperature tolerance is a K to D/E mutation in a direct comparison to any one of the deadCas12a variants of SEQ ID NOs: 14 to 43 as reference sequence, respectively.

In one embodiment, the adenine base editor according to the present invention comprises a dCas12a, or functional fragment thereof, carrying one or more mutations, preferably one to five mutations, especially preferably two to four mutations, conferring an altered PAM specificity as compared to the respective wildtype Cas12a nuclease, or functional fragment thereof, preferably wherein this altered PAM specificity leads to the recognition of one or more PAM sequences selected from the group consisting of TYCV, TATV, TACV, CTCV, CCCV, TTYN, VTTV, and TRTV.

In one embodiment, one of the one or more mutations, preferably one to five mutations, especially preferably two to four mutations, conferring an altered PAM specificity as compared to the respective wildtype Cas12a nuclease, or functional fragment thereof, is a mutation in a dCas12a ortholog or homolog, or a functional fragment thereof, at a position homologous to G532 of SEQ ID NOs: 1, 13, 14, 15, 29, or 30, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 29, or 30.

In one embodiment, one of the one or more mutations, preferably one to five mutations, especially preferably two to four mutations, conferring an altered PAM specificity as compared to the respective wildtype Cas12a nuclease, or functional fragment thereof, corresponds to a mutation in a dCas12a ortholog or homolog, or a functional fragment thereof, at a position homologous to K595 of SEQ ID NOs: 1, 13, 14, 15, 29, or 30, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 29, or 30.

In one embodiment, one of the one or more mutations, preferably one to five mutations, especially preferably two to four mutations, conferring an altered PAM specificity as compared to the respective wildtype Cas12a nuclease, or functional fragment thereof, corresponds to a mutation in a dCas12a ortholog or homolog, or a functional fragment thereof, at a position homologous to K583 of SEQ ID NOs: 1, 13, 14, 15, 29, or 30, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 29, or 30.

In one embodiment, one of the one or more mutations, preferably one to five mutations, especially preferably two to four mutations, conferring an altered PAM specificity as compared to the respective wildtype Cas12a nuclease, or functional fragment thereof, corresponds to a mutation in a dCas12a ortholog or homolog, or a functional fragment thereof, at a position homologous to Y542 of SEQ ID NOs: 1, 13, 14, 15, 29, or 30, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 29, or 30.

Particularly preferably, each of the one or more mutations, preferably one to five mutations, especially preferably two to four mutations, conferring an altered PAM specificity as compared to the respective wildtype Cas12a nuclease, or functional fragment thereof, is selected from the group consisting of G to R, K to R, K to V, and Y to R mutations.

Particularly preferably, the one or more mutations, preferably one to five mutations, especially preferably two to four mutations, conferring an altered PAM specificity as compared to the respective wildtype Cas12a nuclease, or functional fragment thereof, is/are individually selected from the group consisting of G532R, K595R, K538R, and Y542R in relation to any of the sequences according to SEQ ID NOs: 1, 13, 14, 15, 29, or 30, or of a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 1, 13, 14, 15, 29, or 30.

The term “nCas12a” as used herein describes mutants of Cas12a, or functional fragments thereof, showing nickase activity and hence are capable of introducing a single-strand cut (“nick”), preferably with comparable or the same specificity as the respective wildtype Cas12a nucleases introduce double-strand breaks.

In the art, nCas12a variants have been described (e.g. WO2017/127807, WO2019/233990A1, and WO2018/176009). However, to date no nCas12a having functionality in vivo have been reported. Thus, further development can be expected. In view of the fact that nCas9 nickases, which are much easier to create and use in view of the discrete nuclease domains of wildtype Cas9, are very suitable as CBE and ABE elements, any nCas12a can be used as part of an ABE as disclosed herein instead of a dCAs12a in an analogous way.

In one embodiment, the adenine base editor (ABE) may comprise, in sequential order, the following structural elements: a.) at least one N-terminal NLS sequence; b.) an adenosine deaminase domain being selected from a TadA8 or a TadA9 domain, or a functional variant thereof; c.) at least one linker domain, wherein the at least one linker comprises a hexa-GGGGS linker according to SEQ ID NO: 51; d.) a dCas12a, or a functional fragment thereof, or a nCas12a, or a functional fragment thereof; e.) at least one C-terminal NLS sequence; wherein the at least one N-terminal and the at least one C-terminal NLS sequence can be the same or different.

In a preferred embodiment, the adenine base editor (ABE) may comprise, in sequential order, the following structural elements: a.) at least one N-terminal NLS sequence; b.) an adenosine deaminase domain being selected from a TadA8 or a TadA9 domain, or a functional variant thereof; c.) at least one linker domain, wherein the at least one linker comprises a hexa-GGGGS linker according to SEQ ID NO: 51; d.) a dCas12a, or a functional fragment thereof, or a nCas12a, or a functional fragment thereof; e.) at least one C-terminal NLS sequence; wherein the at least one N-terminal and the at least one C-terminal NLS sequence are identical. In one embodiment, the dCas12a or the nCas12a, or the functional fragment thereof, may comprise at least one or more additional mutations as defined above, wherein one of the at least one or more additional mutations conferring enhanced temperature tolerance corresponds to a mutation in a dCas12a ortholog or homolog at a position homologous to position D156 of SEQ ID NO: 14, 15, or 16, or to position E174 of SEQ ID NO: 17, 18, or 19, or to position E184 of SEQ ID NO: 20, 21, 22, 23, 24, 25, 26, 27, or 28, or to a homologous position within a Cas12a ortholog or homolog; preferably wherein one of the at least one or more additional mutations conferring temperature tolerance corresponds to D156R in comparison to SEQ ID NO: 14, 15, or 16 as reference sequences, or at an homologous position within a Cas12a ortholog or homolog, or wherein one of the at least one or more additional mutations conferring temperature tolerance corresponds to E174R in comparison to SEQ ID NO: 17, 18, or 19 as reference sequences, or at an homologous position within a Cas12a ortholog or homolog, or wherein one of the at least one or more additional mutations conferring temperature tolerance corresponds to E184R in comparison to SEQ ID NO: 20, 21, 22, 23, 24, 25, 26, 27, or 28 as reference sequences, or at an homologous position within a Cas12a ortholog or homolog; more preferably wherein the at least one or more additional mutations correspond to (i) D156R and D832A or (ii) D156R and E925A or (iii) D156R and D832A and E925A in comparison to SEQ ID NO: 1 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog, or wherein the at least one or more additional mutations correspond to (iv) E174R and D908A or (v) E174R and E993A or (vi) E174R and D908A and E993A in comparison to SEQ ID NO: 2 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog, or wherein the at least one or more additional mutations correspond to (viii) E184R and D917A or (ix) E184R and E1006A or (x) E184R and D917A and E1006A in comparison to SEQ ID NO: 3 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog.

In one embodiment, the at least one N-terminal NLS sequence and/or the at least one C-terminal NLS sequence is/are selected from a triple SV40 NLS (SEQ ID NO: 52), a bipartite SV40 NLS (SEQ ID NO: 53), a SV40 NLS(SEQ ID NO: 54), a FNLS (SEQ ID NO: 55), or a nucNLS (SEQ ID NO: 56), preferably wherein the at least one N-terminal and the at least one C-terminal NLS sequence is at least one bipartite SV40 NLS (SEQ ID NO: 53), or a functional homolog thereof, or a sequence having at least 95%, 96%, 97%, 98% or at least 99% sequence identity to SEQ ID NO: 53.

In another embodiment, the adenosine deaminase domain may be a TadA8e domain according to SEQ ID NO: 57, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 57.

In another embodiment, the adenosine deaminase domain is a TadA9 domain according to SEQ ID NO: 58, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 58.

In another aspect, the present invention relates to a complex comprising an adenine base editor as described herein and a guide RNA in a functionally associated form, or a sequence encoding the guide RNA, wherein the guide RNA is specific for the dCas12a or for the nCas12a as defined herein, optionally wherein the guide RNA is expressed from a construct comprising a truncated tRNA at the 5′ end and at least one direct repeat structure 5′- and 3′- of the sequence of or encoding the spacer RNA.

The term “complex” as used herein describes an adenine base editor that is functionally associated with at least one guide RNA. The skilled person knows that the nuclease domain of a given adenine base editor is usually non-covalently and reversibly associated with a respective guide RNA.

In the present disclosure, the terms guide RNA and crRNA are used interchangeably. The skilled person in the relevant technical field is aware of the fact that a naturally occurring CRISPR nuclease and the cognate guiding RNA are mutually compatible. Further, the skilled person knows that a different CRISPR/Cas effector is guided by a different type of guiding RNA.

Certain CRISPR nucleases, including Cas9, for example, use a dual heteroduplex guiding RNA (crRNA:tracrRNA), which can also be combined as single guide RNA when used in molecular biology. Other CRISPR nucleases, including the class 2 type V CRISPR nuclease Cas12a, and variants thereof, i.e. a dCas12a or a nCas12a, use a single crRNA RNA as guiding molecule. A guide RNA as used herein is the general term for describing any kind of RNA guiding a CRISPR-nuclease, or a variant thereof. Therefore, when used in the context of a Cas12a effector, the term guide RNA thus refers to a crRNA, or any suitable crRNA-based construct suitable to interact with and guide a Cas12a variant, or an ABE or fusion protein comprising the same, to a target site of interest comprising a suitable PAM.

Advantageously, a construct or nucleic acid molecule for expression of the guide RNA comprising at least one direct repeat structure 5′- and 3′- of the sequence of or encoding the spacer RNA facilitates accurate 3′-end processing of the guide RNA transcript allowing for production of precise guide RNA molecules due to the still intact RNA-processing activity of the dCas12a, or functional fragment thereof and/or the nCas12a, or functional fragment thereof.

The term “spacer RNA” as used herein describes an RNA sequence that is complementary to a specific target region and thus facilitates (i) localization of the respective target region by the complex comprising an adenine base editor as described herein and a functionally associated guide RNA and (ii) binding of the complex to the respective target region.

Preferably, the guide RNA may be expressed from a construct comprising a sequence encoding a spacer RNA, wherein the spacer RNA and the sequence encoding the spacer RNA are 18 to 30 nucleotides in length, preferably 20 to 27 nucleotides in length, especially preferably 21 to 25 nucleotides in length, particularly preferably 22 to 24 nucleotides in length.

The guide RNA may be expressed from a construct comprising a T-stretch terminator 3′- of the direct repeat structure located 3′- of the sequence encoding the spacer RNA. Preferably, this T-stretch terminator consists of 3 to 15, preferably of 4 to 10, especially preferably of 5 to 8 thymine (T) residues.

The terms “thymine” and “thymidine”, e.g. in the context of nucleic acids and/or base editors, are used interchangeably herein.

Pol III, as it is known to the skilled person, terminates transcription at heterogeneous positions within a T-stretch terminator.

Advantageously, an expression construct and/or nucleic acid as described herein comprising a T-stretch terminator located 3′- of the direct repeat structure located 3′- of the sequence encoding the spacer RNA eliminates the drawback of heterogeneous transcription termination by pol 11 as the dCas12a, or functional fragment thereof, or the nCas12a, or functional fragment thereof, cleaves off the direct repeat structure located 3′- of the sequence encoding the spacer RNA together with the poly-U tail transcribed from the T-stretch terminator during processing of the guide RNA utilizing its still intact RNA-processing activity.

The guide RNA may be expressed from a construct comprising at least one Polymerase (pol) 11 promoter. The skilled person knows pol 11 promoters, which are typically used in the art. Preferably, the at least one pol 11 promoter is individually selected from the sequences corresponding to SEQ ID NOs: 69, 70, 71, 72, 73, 74, 75, 76, 77, 78, or 79, or from a sequence having 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 69, 70, 71, 72, 73, 74, 75, 76, 77, 78, or 79.

Particularly preferably, the guide RNA may be expressed from a construct comprising at least one pol III promoter corresponding to SEQ ID NO: 69 or to a sequence having 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 69.

In one embodiment, the guide RNA may be encoded by a scaffold architecture as provided with any one of SEQ ID NO: 59, 60, or 61, or a sequence having at least 90%, 91%, 92%, 93%, 94%, 95%, 96%, 97%, 98% or 99% sequence identity to at least one of the corresponding reference sequences of SEQ ID NO: 59, 60, or 61, respectively.

The term “scaffold architecture” as used herein describes a DNA sequence containing all necessary elements required for transcription of a guide RNA in a way allowing for functional association of a complex comprising an adenine base editor as described herein and the respective guide RNA

The position marked as “n” in any of the sequences corresponding to SEQ ID NOs: 59, 60, or 61 represents a variable region, which encodes for a spacer RNA, and which can be 18 to 30, preferably, 20 to 27, especially preferably 21 to 25, particularly preferably 22 to 24 nucleotides in length, wherein each position can be any nucleotide individually selected from the group consisting of A, G, C, and T.

In a further aspect, the present invention relates to a nucleic acid molecule encoding the adenine base editor as described herein, and/or a nucleic acid molecule encoding the guide RNA as described herein. According to all embodiments associated with a nucleic acid molecule encoding the adenine base editor as described herein, and/or a nucleic acid molecule encoding the guide RNA as described herein, each respective nucleic acid molecules may be codon optimized for expression in a particular species of interest. A particular species of interest may be a plant species, a bacterial species, a fungal species, an archaeal species, or an animal species.

One or more particular prokaryotic species of interested may be selected from the group consisting of Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., and Leptolyngbya sp.

One or more particular eukaryotic microbial species of interested may be selected from the group consisting of Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussu, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger, and Myceliophthora thermophila.

One or more particular plant species of interested may be selected from the group consisting of Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybemum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, and Ziziphus spp.

In yet another aspect, the present invention also relates to an expression construct or a vector comprising a nucleic acid sequence as described herein, wherein the nucleic acid sequence encoding the adenine base editor and/or the nucleic acid sequence encoding the guide RNA are present on the same expression construct or vector, or on at least two individual expression constructs or vectors, optionally wherein an expression construct or vector encoding a guide RNA is present and wherein the guide RNA is expressed from an RNA polymerase 11 promoter or an RNA polymerase II promoter, preferably wherein the promoter is selected from U3, U6, H1, and ubiquitin promoter.

The expression construct or the vector comprising a nucleic acid sequence as described herein, wherein the nucleic acid sequence encoding the adenine base editor and/or the nucleic acid sequence encoding the guide RNA are present, may comprise at least one pol 111 promoter. The skilled person knows pol 111 promoters, which are typically used in the art.

Preferably, the at least one pol 11 promoter is individually selected from the sequences corresponding to SEQ ID NOs: 69, 70, 71, 72, 73, 74, 75, 76, 77, 78, or 79, or from a sequence having 80%, 81%, 82%, 83%, 84%, 85%, 86%, 87%, 88%, 89%, 90%, 91%, 92%, 93%, 94%, 95%, 96%, 97%, 98% or at least 99% sequence identity to any of the sequences corresponding to SEQ ID NOs: 69, 70, 71, 72, 73, 74, 75, 76, 77, 78, or 79.

Preferably, the expression construct or the vector comprising a nucleic acid sequence as described herein, wherein the nucleic acid sequence encoding the adenine base editor and/or the nucleic acid sequence encoding the guide RNA are present, comprises at least one pol 11 promoter corresponding to SEQ ID NO: 69 or to a sequence having 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 69.

In a further aspect, the present invention also relates to a cell comprising an adenine base editor as described herein, or comprising a nucleic acid sequence encoding the complex as described herein, or comprising an expression construct or a vector of as described herein.

In another aspect, the present invention also relates to a method of adenine base editing of a target site in a genome of interest in at least one cell of a prokaryotic or eukaryotic organism, the method comprising the following steps: (a) providing at least one adenine base editor or at least one complex as described herein, or a nucleic acid molecule or expression construct encoding the same as described herein, to the at least one cell; (b) optionally: allowing functional expression and/or assembly of a complex into a functionally associated form as defined herein; (c) contacting the genome of interest of the at least one cell with at least one functionally associated form of a complex comprising at least one adenine base editor or at least one complex as described herein to obtain at least one modified cell; (d) optionally: selecting the at least one modified cells; and (e) obtaining at least one cell containing at least one adenine base edit at the target site, wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes and further wherein the method excludes the treatment of a human or animal body by therapy or surgery,

Optionally, where the method comprises the following step:

(f) regenerating at least one population of edited cells, tissues, organs, materials or whole organisms from the at least one edited cell.

Preferably, the at least one cell may be from a plant, algae, yeast or fungus organism, preferably wherein the at least one cell is a plant cell, preferably a plant cell belonging to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus offlicinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybemum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

The term “plant” as used herein encompasses whole plants, ancestors and progeny of the plants and plant parts, including seeds, shoots, stems, leaves, roots (including tubers), flowers, and tissues and organs. Further disclosed in the context of plants are plant cells, suspension cultures, callus tissue, embryos, meristematic regions, gametophytes, sporophytes, pollen and microspores from a plant that can be obtained, analyzed, treated in line with the disclosure provided herein.

Plants that are particularly useful in the methods of the invention include all plants which belong to the superfamily Viridiplantae, in particular monocotyledonous and dicotyledonous plants. In one embodiment the method of the invention relates to the use of fodder or forage legumes, ornamental plants, food crops, trees or shrubs. For example, the method of the invention relates to the use of crop plants, e.g. like the crop plants listed below. The plant can be selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Omithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybemum, Triticum macha, Triticum sativum, Triticum monococcum or Triticum vulgare), Tropaeolum minus, Tropaeolum majus, Vaccinium spp., Vicia spp., Vigna spp., Viola odorata, Vits spp., Zea mays, Zizania palustris, Ziziphus spp., amongst others.

Preferred plants are Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybemum, Triticum macha, Triticum sativum, Triticum monococcum or Triticum vulgare), Zea mays.

Especially preferred plants are Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybemum, Triticum macha, Triticum sativum, Triticum monococcum or Triticum vulgare), Zea mays.

In a further aspect, the present invention also relates to an edited cell, or a tissue, organ, material (e.g., a material from a leaf, or from a germ cell, or part of an organ, or part of a seed, for example, in crushed form etc.) or whole organism obtained by or obtainable by a method as described herein. As all methods and uses disclosed herein specifically exclude processes for modifying the germ line genetic identity of human beings, uses of human embryos for industrial or commercial purposes, processes for cloning human beings, and further processes for modifying the genetic identity of animals which are likely to cause them suffering without any substantial medical benefit to man or animal, and also animals resulting from such processes, the edited cell does not comprise a human germ line cell or any human embryos. The methods and uses disclosed herein, however, specifically refer to uses and methods using non-embryo and non-germ line human cells, e.g., primary human cells like macrophages, T-cells and the like, that are edited ex vivo under in vitro conditions.

Further, in all aspects and embodiments as disclosed herein, a method or use as described herein, as far as it refers to a plant cell, comprises that said at least one plant cell, tissue, organ, plant, or seed is not obtained by an essentially biological process. Instead, said at least one plant cell, tissue, organ, plant, or seed is obtained by at least one step of artificial human intervention in the form of using an ABE as disclosed herein as such not occurring in nature and influencing the plant cell by modifying and/or introducing a step of technical nature influencing sexually crossing and selecting. Such a step may include a step of genome editing, e.g., to exchange a base or nucleotide of interest, a chemical treatment, e.g. for chromosome doubling, an agent or gene or gene product including chromosome elimination, the introduction of an exogenous gene or genetic material into a plant genome (nuclear, mitochondrial or plastid genome) and the like, or any combination thereof.

In yet another aspect, the present invention also relates to a kit comprising (a) the adenine base editor as described herein and/or the complex as described herein and/or a nucleic acid molecule as described herein and/or an expression construct as described herein and/or a cell as described herein, and comprising (b) a container containing reaction components including buffers and optionally comprising (c) instructions for use.

According to all embodiments related to a kit as described herein the reaction components including buffers provide suitable reaction conditions to promote the activity of the adenine base editor as described herein and/or the complex as described herein and/or a nucleic acid sequence as described herein and/or an expression construct as described herein and/or a cell as described herein.

In yet a further aspect, there is provided a method of obtaining a plant or seed thereof, or progeny thereof regenerated from the plant or seed, wherein the method may comprise the propagation of the trait introduced by at least one adenosine base editor as described herein into the at least one genomic target site, i.e., the site to be modified and/or the site the ABE interacts with, of the plant or seed thereof. In one embodiment, the genome of the plant or seed modified by at least one targeted edit as mediated by the at least one adenosine base editor as described herein can thus be used to modify the genome of a progeny in a targeted way by specifically combining the genomes of originally polyploid plants, so that the at least one targeted edit will be present at at least allele in the progeny.

In one aspect, there may be provided the use of an adenine base editor, of a complex, or of an expression construct or vector as described herein for adenine base editing of a target site in a genome of interest in at least one cell of a prokaryotic organism, including bacterial and archaeal organisms, or eukaryotic organism.

### EXAMPLES

### Example 1: Molecular Methods

### Example 1.1: Cloning

PCR was performed using Q5® High-Fidelity DNA Polymerase (M0491, NEB) with DNA oligonucleotides from Integrated DNA Technologies (IDT). PCR products were gel purified using Gel Purification Kit (Zymo Research, no. D4002). To generate entry vectors, DNA fragments were inserted into Bsal-digested GreenGate empty entry vectors via Gibson assembly (2×NEBuilder Hifi DNA Assembly Mix, NEB) or restriction ligation with T4 DNA ligase (NEB). Base editors, gRNAs and fluorescent reporter vectors were assembled using Golden Gate cloning (30 cycles (37° C., 5 min; 16° C., 5 min); 50° C. for 5 min; 80° C. for 5 min) with Bsal or Bbsl. Vectors were transformed by heat-shock transformation into DH5a E. coli or One Shot™ ccdB Survival™ competent cells (Thermo Fisher Scientific). Cells were plated on lysogeny broth medium containing 100 μg mL⁻¹ carbenicillin, 100 μg mL⁻¹ spectinomycin, 25 μg mL⁻¹ kanamycin or 40 μg mL⁻¹ gentamycin depending on the selectable marker. Plasmids were isolated (GeneJET Plasmid Miniprep kit, Thermo Fisher Scientific) and confirmed by restriction enzyme digestion and/or Sanger sequencing (Eurofins, Mix2seq).

### Example 1.2: Entry Clones

TadA7.10d was synthesized on the BioXP3200 DNA synthesis platform (Codex DNA) based on the published sequence (Gaudelli et al., Nature 551, 464-471, 2017), TadA8e (#138489) and Tad8.20m (#136300) were ordered from Addgene.

The LbCas12a(D832A) sequence was codon-optimized for wheat and subsequently synthesized (Twist Biosciences). Three synthesized fragments were cloned into an entry vector using Gibson assembly. The LbCas12a(D156R-D832A) variant was generated by site-directed mutagenesis PCR via Gibson assembly. The 3×SV40-NLS and BPstar-NLS sequences were previously published (Richter at el., Nature Biotechnology 38, 883-891, 2020; Alok et al., Frontiers in Plant Science 11, 264, 2020) and were cloned by annealed oligos followed by ligation. Nuc-UGI-SV40 was amplified from A3A-PBE (Addgene #119768); and cloned into an entry vector via Gibson assembly. The CaMV terminator was isolated from the PABE-7 plasmid (Addgene #115628) and cloned into an entry vector via Gibson assembly.

### Example 2: Plant Growth Conditions

Wheat seeds (Fielder) were processed by successive washes with sterilized water for 3 min, isopropanol for 45 sec, sterile water for 3 min and 6% sodium hypochlorite (Chem-lab nv) for 10 min. Sterilized seeds were washed six times with sterile water in a laminar flow cabinet and sown on sterile growth media containing ½ MS pH 5.7 (Duchefa Biochemie, M0221.0050), 2.5 mM MES (Duchefa Biochemie, M1503.0100) and 0.5% plant tissue agar (NEOGEN, NCM0250A). Two seeds were sown per sterile, 175 ml cylindrical container (Greiner Bio-one, #960162) and stratified for 3 days at 4° C. in the dark. Plants were grown under SpectraluxPlus NL 36 W/840 Plus (Radium Lampenwerk) fluorescent bulbs under long days (16h light/8h dark) at 25° C.

B104 maize seeds were sown directly on Jiffy substrate (Jiffy Products International, No. 32170138). Seed germination was performed in long day (16h light/8h dark) conditions at 25° C., 55% relative humidity for 5 days under light provided by high-pressure sodium vapor (RNP-T/LR/400W/S/230/E40; Radium) and metal halide lamps with quartz burners (HRI-BT/400W/D230/E40; Radium). Seedlings were transferred to the dark for 8 days prior to protoplast isolation.

### Example 3: Protoplast Isolation and Transfection

### Example 3.1: Wheat Protoplast Isolation

Wheat leaves were harvested 7 or 8 days after germination (DAG). Approximately 40-50 second leaves were cut into latitudinal 0.5-1 mm strips with a sharp razor blade and leaf strips were incubated in 0.6 M D-mannitol (Sigma-Aldrich, M1902) for 10 min in the dark. The mannitol was removed and 25 ml cell wall enzyme solution (20 mM MES, 1.5% cellulase R10 (C8001.0010), 0.75% macerozyme R10 (M8002.0010), 0.6 M D-mannitol and 10 mM KCl, 0.1% BSA and 10 mM CaCl₂) was added to protoplasts for 8 hours incubation in the dark at 25° C. with 40 rpm shaking. After enzymatic digestion, 25 ml of W5 solution (2 mM MES pH 5.7, 154 mM NaCl, 125 mM CaCl2 0.5 mM KCl) was added to release the protoplasts. Protoplasts were collected by filtering the mixture through a sterile 40 μm cell strainer (Corning, #431750) and centrifugation at 80 g (slow acceleration and brake) for 3 min at room temperature. The supernatant was discarded and protoplasts were resuspended in 6 ml W5 solution and incubated on ice for 30 min. Protoplast yield was determined using a Neubauer chamber before adding MMGTa solution (4 mM MES pH 5.7, 0.4 M mannitol, 15 mM MgCl₂) onto the cell pellet to reach a concentration of 1×106 cells ml-1.

### Example 3.2: Wheat Protoplast Transfection

Protoplasts were then incubated on ice for ˜30 min before transfection. 12 μg of total plasmid DNA was added to MMGTa to a total volume of 20 μl in 1 ml strip tubes (National Scientific Supply Co, TN0946-08B). 100 μl of protoplasts (105 cells) and 110 μl of PEG solution (0.2 M mannitol, 100 mM CaCl₂), 40% PEG (Sigma 81240) were added using a multichannel pipette to DNA and immediately mixed by slowly inverting the strip. For individual strips, 8 transfections were processed in parallel. Protoplasts were incubated for 15-20 min and W5 solution was added to stop the transfection. After centrifugation at 80 g (slow acceleration and brake) for 3 min, the supernatant was discarded and the protoplast pellet resuspended in 1 ml of W5 solution. Cells were then transferred in 24-well plates (VWR 734-2325 EU catalog) and incubated in the dark at 25° C. for 42 to 46 hours.

### Example 3.3: Maize Protoplast Isolation

Etiolated maize leaves were harvested at 12 or 13 DAG. The middle part of the second or third leaf was cut into 0.5 mm strips. Strips were then infiltrated with 25 ml cell wall enzyme solution (0.6 M D-mannitol, 10 mM MES, 1.5% cellulose, 0.3% Macerozyme R10, 0.1% BSA and 1 mM CaCl2) using vacuum (50 mmMg) for 30 minutes in the dark and then incubated for 2 hours at 25° C. with shaking (40 rpm). The solution containing the protoplasts was filtered using a sterile 40 μm cell strainer (Corning) and collected by centrifugation at 100 g (slow acceleration and brake) for 3 min. The supernatant was removed and protoplasts were washed with ice-cold 0.6 M D-mannitol by centrifugation at 100 g (slow acceleration and brake) for 2 min. Cells were then resuspended in 5 ml of 0.6 M D-mannitol and incubated in the dark for 30 min. Protoplasts were resuspended in MMGZm solution (0.6 M mannitol, 15 mM MgCl2, 4 mM MES) and counted using a Neubauer chamber and adjusted to a concentration of 1×106 cells ml-1.

### Example 3.4: Maize Protoplast Transfection

20 μg of total plasmid DNA was added to MMGZm to a total volume of 20 μl in 1 ml strip tubes. 100 μl of protoplasts (105 cells) and 110 μl of PEG (0.2 M mannitol, 100 mM CaCl2), 40% PEG (Sigma 81240) solution were added using a multichannel pipette to DNA and immediately mixed by inverting the strip. For individual strips, 8 transfections were processed in parallel. Cells were then incubated for 10-15 min in the dark and W5 solution was added to stop the transfection. After centrifugation at 100 g (slow acceleration and brake) for 2 min, supernatant was discarded and the protoplast pellet resuspended in 1 ml of W5 solution. Cells were then transferred using tips with wide bore in 24-well plates (VWR) and incubated in the dark at 25° C. with shaking (20 rpm) for 2 days.

### Example 4: High Content Image Analysis

Two days after transfection, 50 μl of protoplasts were transferred to 96-well Cell carrier Ultra plates (#6055302) and imaged with the Opera Phenix® High Content Screening System (PerkinElmer). Image acquisition was performed using a 20× water immersion objective in confocal mode, taking 7 Z-planes and 9 fields of view per well and covering 4 image channels: brightfield, Chlorophyl, GFP and mCherry. Raw images were transferred to the Columbus™ Image Data Storage and Analysis system for automated image processing and quantification.

After flatfield correction and smoothing of the chlorophyll channel, single wheat cells were segmented and selected as protoplasts based on roundness. mCherry and GFP signals were used to identify nuclei and to exclude non-transformed protoplasts based on the absence of nuclear mCherry signal. The mCherry and GFP intensities in the nuclei of transformed protoplasts were used to identify and quantify the GFP expressing transformed protoplasts.

For maize, the chlorophyll channel could not be used for cell segmentation as the plants were etiolated. The analysis focused directly on transformed protoplast nuclei, segmenting based on mCherry and GFP channels. The same analysis as described above was used to identify and quantify the GFP expressing transformed nuclei.

Results were exported as a table and all calculations and image processing were performed on an in-house cluster (VIB). The time required from the start of imaging to obtaining processed results takes 3-4 hours for a 96-well plate. Codes for wheat and maize analysis workflows are available in supplemental data.

### Example 5: FACS

Images were captured using a BD Biosciences FACS imaging enabled prototype cell sorter that is equipped with an optical module allowing multicolor fluorescence imaging of fast flowing cells in a stream enabled by BD CellView™ Image Technology based on fluorescence imaging using radiofrequency-tagged emission (FIRE).

Two days after transfection, 500 μl of protoplast solution was used for sorting. Gating strategies for GFP were first established on cells expressing pZmUBI-GFP-NLS (p02243) and similar settings were used for all experiments in wheat and maize. A quality check was conducted by running the sorted cell fraction on the instrument and imaged using the imaging system integrated in the FACS instrument. For both wheat and maize, a 130 μm nozzle was used and 1,000 to 5,000 cells were sorted into 1.5 ml Eppendorf tubes containing 10 μl of dilution buffer from the Phire Tissue Direct PCR Master Mix kit (Thermo Fisher Scientific, F160L).

### Example 6: Genotyping and NGS Analysis

For genotyping individual wheat transformed plants, a piece of leaf (0.5-1 cm) was harvested in 1 ml tubes on 96-well plate (VWR, 732-3716) and flash frozen in liquid nitrogen. Two metal beads (3 mm) were added and tissue was ground to powder by shaking the plate at 20 Hz for 1 minute (Retsch, Mixer Mill MM 400). 400 μl of extraction buffer (100 mM Tris-HCl pH 8.0, 500 mM NaCl, 50 mM EDTA, 0.7% SDS) was added to individual samples and incubated 30 min at 60° C. Samples were centrifuged and 300 μl of the supernatant was mixed to 300 μl of isopropanol for DNA precipitation. Samples were then centrifuged and supernatant removed. The pellet was washed with 70% Ethanol, dried at room temperature, and dissolved in 100 μl of 10 mM Tris-HCl pH=8.0.

For sorted material, 2 μl of the solution containing sorted cells was used as template in a 20 μl total reaction volume for amplicon PCR using the Phire Plant Direct PCR Kit (Thermo Fisher Scientific, F160L) according to manufacturer's recommendations.

For dCas12-BE and nuclease-active LbCas12a, base editing and indel efficiencies were measured using NGS. 210-260 bp amplicons were designed to amplify target sites. 6-nt indices were added to forward and reverse primers for pooling and demultiplexing amplicon reads after sequencing. 5 μl of the Phire PCR reaction was verified on a 2% agarose gel with a low molecular weight ladder (NEB, no. N3233S). 15 μl of PCR products were pooled and purified using PCR Purification Kit (Zymo Research Co., D4013). Depending on the amplicon, an extra gel purification was conducted (Zymo Research, D4002) to specifically isolate the PCR band of the target site. The DNA concentration was measured with Qubit (Invitrogen) according to manufacturer's protocol and adjusted to 2 ng μl⁻¹. Paired end sequencing was performed with Eurofins NGSelect amplicons (5M reads 2×150 bp). Reads were demultiplexed using Je-demultiplex and individual fastq files were obtained using a Galaxy workflow (https://usegalaxy.be).

Base editing was calculated using CRISPResso2Pooled or CRISPRessoBatch. Editing window and read quality were defined as follows: cleavage offset was set to −1, quantification window size to 10, quantification window center to −12 and minimum average read quality to 30. Indels were calculated using CRISPResso2Pooled or CRISPRessoBatch with the following settings to define Cas12a cutting site and read quality: cleavage offset was set to −4 and minimum average read quality to 30.

### Example 7: Stable Wheat Transformation

Immature embryos 2-3 mm in size were isolated from sterilized ears of wheat cv. Fielder and bombarded using the PDS-1000/He particle delivery system (Bio-Rad) using the following particle bombardment parameters: diameter gold particles, 0.6 μm; target distance, 6 cm; bombardment pressure, 7.584 kPa; gap distance, 8-10 mm; microcarrier flight distance, 10 mm; vacuum within the bombardment chamber, 27.5″ Hg. For each shot approximately 150 μg of gold particles carrying 570 ng of plasmid DNA were delivered.

The applied plasmid DNA was a mixture of the Cas12a-ABE vectors pCG392 or pCG434, pCG406 and pCG408 (gRNAs) and pBAY02032 (selectable marker). The vector pBAY02032 contains an eGFP-BAR fusion gene under control of the 35S promoter. Bombarded immature embryos were transferred to non-selective WLS callus induction medium for about one week, then moved to WLS with 5 mg L-1 phosphinothricin (PPT) for a first selection round of about 3 weeks followed by a second selection round on WLS with 10 mg L⁻¹PPT for another 3 weeks. PPT resistant calli were selected and transferred to shoot regeneration medium with 5 mg L⁻¹PPT.

### Example 8: Iterative Testing of Cas12-ABE Components in Wheat

The different components of the Cas12a-ABE were subjected to extensive iterative testing in wheat protoplasts to develop an optimized Cas12a-ABE architecture.

Wheat protoplast isolation was performed according to Example 3.1: Wheat leaves were harvested 7 or 8 days after germination. Approximately 40-50 second leaves were cut into latitudinal 0.5-1 mm strips with a sharp razor blade and leaf strips were incubated in 0.6 M D-mannitol (Sigma-Aldrich) for 10 min in the dark. The mannitol was removed and 25 ml cell wall enzyme solution (20 mM MES, 1.5% cellulase R10, 0.75% macerozyme R10, 0.6 M D-mannitol and 10 mM KCl, 0.1% BSA and 10 mM CaCl2) was added to protoplasts for 8 hours incubation in the dark at 25° C. with 40 rpm shaking. After enzymatic digestion, 25 ml of W5 solution (2 mM MES pH 5.7, 154 mM NaCl, 125 mM CaCl₂) 0.5 mM KCl) was added to release the protoplasts. Protoplasts were collected and centrifuged at 80 g for 3 min at room temperature. The supernatant was discarded and protoplasts were resuspended in 6 ml W5 solution and incubated on ice for 30 min. Protoplast yield was determined using a Neubauer chamber before adding MMG solution (4 mM MES pH 5.7, 0.4 M mannitol, 15 mM MgCl₂) onto the cell pellet to reach a concentration of 1×10⁶ cells ml⁻¹.

Wheat protoplast transfection was performed according to Example 3.2: Protoplasts were then incubated on ice for +/−30 min before transfection. 12 μg of total plasmid DNA was added to MMG to a total volume of 20 μl in 1 ml strip tubes (National Scientific Supply Co). 100 μl of protoplasts (=1×10⁵ cells) and 110 μl of PEG solution (0.2 M mannitol, 100 mM CaCl₂, 40% PEG (Sigma 81240) were added using a multichannel pipette to DNA and immediately mixed by slowly inverting the strip. For individual strips, 8 transfections were processed in parallel. Protoplasts were incubated for 15-20 min and W5 solution was added to stop the transfection. After centrifugation at 80 g for 3 min, the supernatant was discarded and the protoplast pellet resuspended in 1 ml of W5 solution. Cells were then transferred in 24-well plates and incubated in the dark at 25° C. for 42 to 46 hours. For all examples 1.1 to 1.2 a wheat codon optimized version of LbCas12a was used.

In total, 12 different expression constructs comprising a nucleic acid sequence encoding an adenine base editor as described herein were constructed (see FIG. 1a; constructs 1 to 12). Besides that, a total of 8 different expression constructs comprising a nucleic acid sequence encoding a guide RNA as described herein were constructed (see FIG. 1b; constructs a to h). To test for ABE activity, wheat protoplasts were co-transfected with 3 vectors: (1) a vector encoding a mutated GFP gene in which a Gln (Q) codon (CAG) was mutated into a stop codon (TAG) (FIG. 1c), (2) a Cas12a-ABE expression vector carrying a p35S:mCherry-NLS cassette (3) a vector encoding a gRNA targeting the mutated GFP codon. Two days after transfection, protoplasts were transferred to 96-well Cell carrier Ultra plates and imaged with the Opera Phenix® High Content Screening System (Perkin Elmer). Image acquisition was performed using a 20× water immersion objective in confocal mode, taking 7 Z-planes and 9 fields of view per well and covering 4 image channels: brightfield, Chlorophyl, EGFP and mCherry. Raw images were transferred to the Columbus™ Image Data Storage and Analysis system for automated image processing and quantification. After flatfield correction and smoothening of the chlorophyll channel, single wheat cells were segmented and selected as protoplasts based on roundness. Editing of the TAG codon into a CAG codon restores the GFP coding sequence and results in GFP fluorescence. The ratio of GFP cells/mCherry cells [%] was determined as a measure for ABE activity. Thus, a higher ratio of GFP cells/mCherry cells indicates higher ABE activity.

### Example 8.1: Evaluation of the Adenosine Deaminase Domain

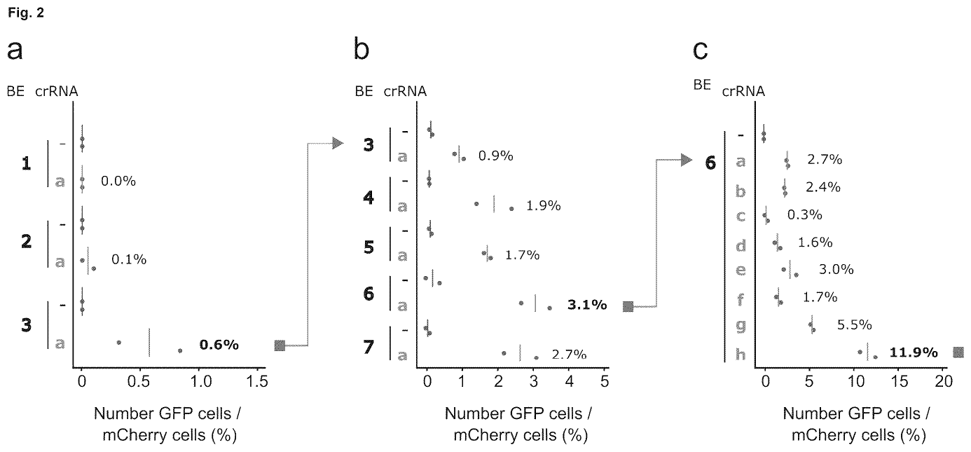

Three different expression constructs comprising a nucleic acid sequence encoding an adenine base editor as described herein were comparatively analyzed with respect to their ABE activity, as each of these expression constructs contained a different adenosine deaminase domain as the sole distinguishing feature: (i) TadA7.10d (construct 1; see FIG. 1a), (ii) TadA8.20 (construct 2; see FIG. 1a), (iii) TadA8e (construct 3; see FIG. 1a). The respective ABE activities were tested in wheat protoplasts and expressed as GFP cells/mCherry cells [%](see FIG. 2a). Each of the three different adenine base editor expression constructs was co-transfected with the same guide RNA expression construct (construct a; see FIG. 1b). As a negative control, all three adenine base editor expression constructs were tested without a guide RNA expression construct (see FIG. 2a).

The results show that ABE activity without any guide RNA was not detectable in all cases (see FIG. 2a). ABE activity with TadA8e as adenosine deaminase (0.6%) was consistently higher as compared to the other two tested adenosine deaminase domains (0.0% and 0.1% for TadA7.10d and TadA8.20, respectively; see FIG. 2a).

### Example 8.2: Evaluation of the NLS Sequence

Five different expression constructs comprising a nucleic acid sequence encoding an adenine base editor as described herein were comparatively analyzed with respect to their ABE activity. Each of these expression constructs contained a different combination of NLS sequence configuration and plant terminator sequence: (i) one 3×SV40 (SEQ ID NO: 52) 3′- of the dLbCas12a domain (construct 3; see FIG. 1a), (ii) one BP (SEQ ID NO: 53) 5′- of the adenosine deaminase domain and one 3×SV40 (SEQ ID NO: 52) 3′- of the dLbCas12a domain (constructs 4 [G7T terminator] and 5 [CaMV terminator]; see FIG. 1a), (iii) one BP (SEQ ID NO: 53) 5′- of the adenosine deaminase domain and one BP (SEQ ID NO: 53) 3′- of the dLbCas12a domain (constructs 6 [G7T terminator] and 7 [CaMV terminator]; see FIG. 1a). The respective ABE activities were tested in wheat protoplasts and expressed as GFP cells/mCherry cells [%](see FIG. 2b). Each of the different adenine base editor expression constructs were co-transfected with the same guide RNA expression construct (construct a; see FIG. 1a). As a negative control, all adenine base editor expression constructs were tested without a guide RNA expression construct (see FIG. 2b).

The highest ABE activity was found for constructs 6 and 7 (3.1% and 2.7% respectively; see FIG. 2b), comprising one BP (SEQ ID NO: 53) 5′- of the adenosine deaminase domain and one BP (SEQ ID NO: 53) 3′- of the dLbCas12a domain. The other constructs yielded ABE activities ranging from 0.9% to 1.9% (construct 3 to construct 5; see FIG. 2b). The impact of the terminator sequence on ABE activity was not significant.

### Example 8.3: Evaluation of the Guide RNA System

The adenine base editor expression construct 6 (see Example 1.2) was tested in combination with eight different guide RNA expression constructs (constructs a to h; see FIG. 1b). The respective ABE activities were determined in wheat protoplasts and expressed as GFP cells/mCherry cells [%](see FIG. 2c). As a negative control, the adenine base editor expression construct 6 was tested without a guide RNA expression construct (see FIG. 2c).

Adenine base editor expression construct 6 yielded the highest ABE activity in combination with guide RNA expression construct h (11.9%; see FIG. 2c), which comprised (i) a truncated tRNA 5′- of the first of two mature direct repeat sequences, (ii) one mature direct repeat sequence 5′- of the sequence encoding for the spacer RNA, (iii) a second mature direct repeat sequence 3′- of the sequence encoding for the spacer RNA, (iv) a poly-T tail (T-stretch terminator). In combination with adenine base editor expression construct 6, the other tested guide RNA expression constructs yielded ABE activities ranging from 0.3% to 5.5% (construct c to g; see FIG. 2c).

### Example 8.4: Evaluation of the Cas12a Domain

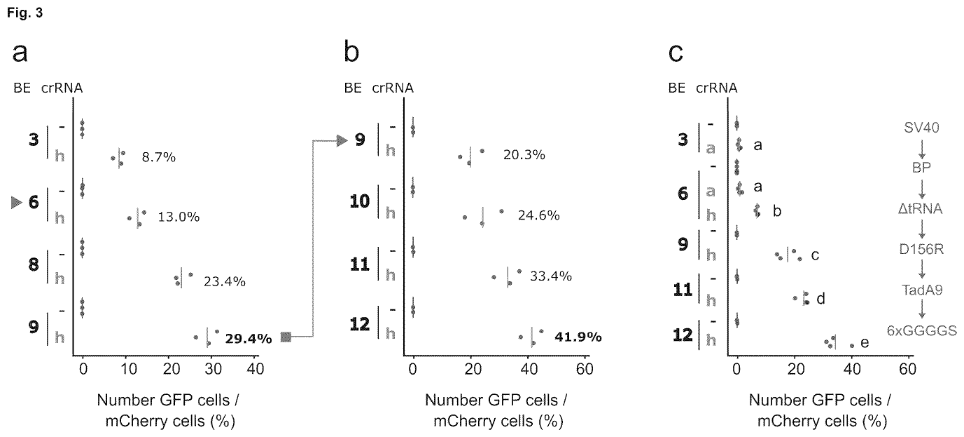

Four different adenine base editor expression constructs (constructs 3, 6, 8, and 9; see FIG. 1a) were tested in combination with guide RNA expression construct h (see FIG. 1b). The respective ABE activities were determined in wheat protoplasts and expressed as GFP cells/mCherry cells [%](see FIG. 3a). As a negative control, all adenine base editor expression constructs were tested without a guide RNA expression construct (see FIG. 3a).

For adenine base editor expression constructs 8 and 9 significantly higher ABE activities (23.4% and 29.4%, respectively; see FIG. 3a) were determined as compared to adenine base editor expression constructs 3 and 6 (8.7% and 13.0%, respectively; see FIG. 3a). In contrast to constructs 3 and 6 (both comprising dLbCas12a), constructs 8 and 9 comprised the D156R mutant of dLbCas12a displaying increased activity and/or enhanced temperature tolerance as compared to the wildtype LbCas12a enzyme (Schindele and Puchta, 2020, Plant Biotechnol. J., 18(5), p. 1118-1120. doi: https://doi.org/10.1111/pbi.13275). The highest ABE activity was detected for construct 9 (29.4%) comprising as the C-terminal NLS sequence one BP (SEQ ID NO: 53) 3′- of the dLbCas12a domain (see FIG. 1a). In contrast, construct 8 comprised as a C-terminal NLS sequence one 3×SV40 (SEQ ID NO: 52) 3′- of the dLbCas12a domain (see FIG. 1a).

### Example 8.5: Evaluation of TadA8e Vs. TadA9 and 32Aa Linker Vs. Hexa-GGGGS Linker

Four different adenine base editor expression constructs (constructs 9, 10, 11, and 12; see FIG. 1a) were tested in combination with guide RNA expression construct h (see FIG. 1b).

The respective ABE activities were tested in wheat protoplasts and expressed as GFP cells/mCherry cells [%](see FIG. 3b). As a negative control, all adenine base editor expression constructs were tested without a guide RNA expression construct (see FIG. 3b).

Overall, both constructs (constructs 11 and 12; see FIG. 1a) comprising TadA9 as an adenosine deaminase domain yielded higher ABE activities as compared to both constructs comprising TadA8e as an adenosine deaminase domain (constructs 9 and 10). The highest ABE activity (41.9%) was determined for construct 12 (see FIG. 3b) comprising TadA9 as an adenosine deaminase domain and a Hexa-GGGGS linker domain connecting TadA9 to dLbCas12a (D156R mutant) domain located 3′- of TadA9 (see FIG. 1a). In contrast, construct 11 comprised a 32aa linker domain (see FIG. 1a).

### Example 8.6: Comparative Analysis of Cas12a-ABEs in Wheat

We compared the successive BE architectures in a single wheat protoplast experiment to confirm that individual modifications along the optimization path of LbCas12a-ABE led to increased activity (FIG. 3c). In line with our previous experiments, introducing the truncated tRNA DR-DR crRNA system, the LbCas12a(D156R) variant, TadA9 and the 6×GGGGS linker all led to significant increases in base editing efficiencies (One-way ANOVA, Tukey HSD: P<0.05).

### Example 9: Comparative Analysis of Cas12a-ABEs in Maize

Six different combinations of adenine base editor expression constructs and guide RNA expression constructs were comparatively analyzed in maize protoplasts. The respective ABE activities were expressed as GFP cells/mCherry cells [%](see FIG. 4b). As a negative control, all adenine base editor expression constructs were tested without a guide RNA expression construct (see FIG. 4b).

The results show that introduction of an N- and C-terminal BP (SEQ ID NO: 53) as NLS in combination with the guide RNA architecture comprising a truncated tRNA flanked by a mature direct repeat sequence in 5′- and 3′- orientation (see construct h; FIG. 1b) leads to a first pronounced increase in editing efficiency (v6h with 13.3% vs. v3a with 0.8% and v6a with 0.2%; see FIGS. 4a and b). Additionally introducing a mutation conferring enhanced temperature tolerance to the dLbCas12a domain (D156R) leads to a further 2-fold increase in editing efficiency from 13.3% (construct v6h) to 26.1% (construct v9h; see FIGS. 4a and b). Furthermore, replacing the TadA8e adenosine deaminase domain with TadA9 further leads to an almost 2-fold increase in editing efficiency (47.7% for construct v11h; see FIGS. 4a and b). Finally, replacing the 32aa linker domain with a Hexa-GGGGS linker domain increases editing efficiency by another 20% (67.9% for construct v12h; see FIGS. 4a and b).

### Example 10: Validation of Cas12a-ABE Activity at Endogenous Target Sites in Wheat and Maize

### Example 10.1: Cas12a-ABE activity at endogenous target sites in wheat (Triticum aestivum)

Base editing activity of optimized LbCas12a-ABE was determined by measuring A to G substitutions at endogenous wheat genome sites. Transfected protoplast samples were sorted using a BD Biosciences FACS imaging enabled prototype cell sorter according to Example 5. Two days after transfection, 500 μl of protoplast solution was used for sorting. Gating strategies for GFP were first established on cells expressing pZmUBI-GFP-NLS and similar settings were used for all experiments in wheat and maize. For both wheat and maize, a 130 μm nozzle was used and 1,000 to 5,000 cells were sorted into 1.5 ml Eppendorf tubes containing 10 μl of dilution buffer from the Phire Tissue Direct PCR Master Mix kit (Thermo Fisher Scientific). 2 μl of the solution containing sorted cells was used as template in 20 μl total reaction volume for amplicon PCR using the Phire Plant Direct PCR Kit according to manufacturer's recommendations. PCR products were pooled and purified using PCR Purification Kit (Zymo Research Co., D4013). Paired end sequencing was performed with Eurofins NGSelect amplicons (5M reads 2×150 bp). Reads were demultiplexed using Je-demultiplex (Girardot et al., 2016 BMC Bioinformatics 17, 419) and individual .fastq files were obtained as previously described (Bollier et al., 2020 BioRxiv 11.13.381046) using a Galaxy workflow (https://usegalaxy.be). Base editing was calculated using CRISPResso2Pooled or CRISPRessoBatch (Clement et al., 2019 Nature Biotechnology 37, 224-226).

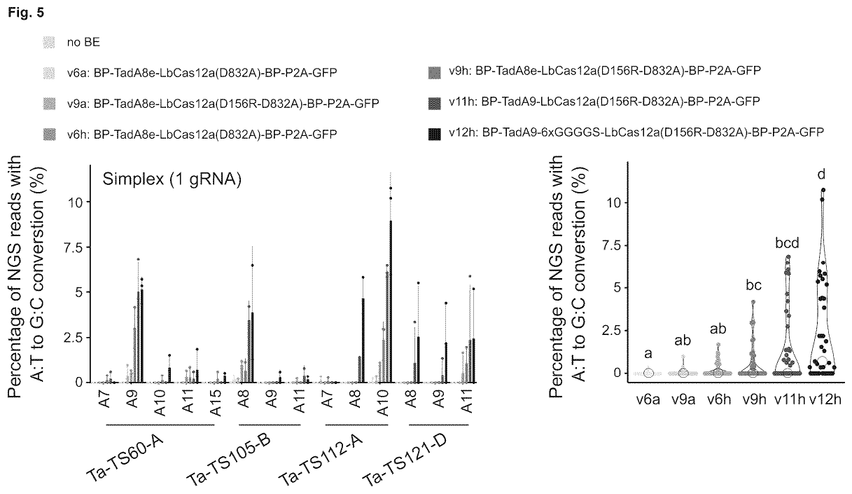

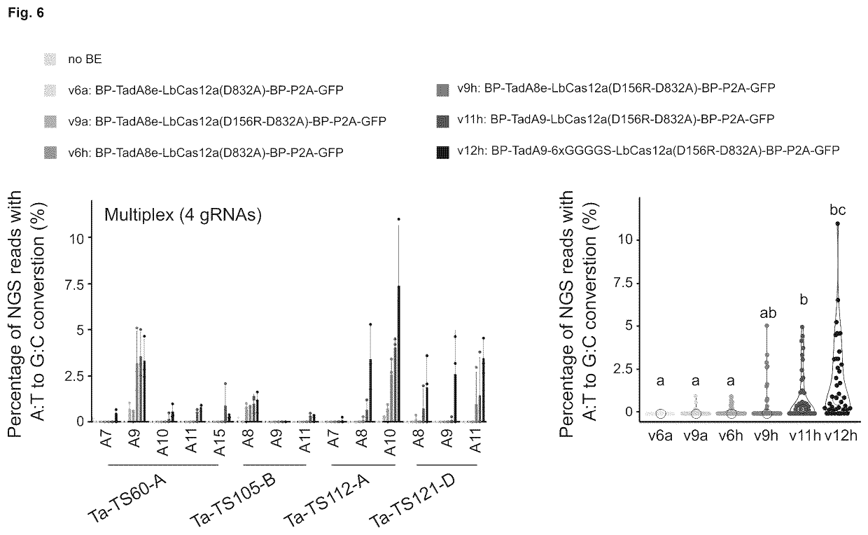

Six LbCas12a-ABE and crRNA architectures were selected (numbers and letters according to nomenclature of FIGS. 1a and b) and their respective ABE activity was determined at four sites (Ta-TS60-A, Ta-TS105-B, Ta-TS112-A, and Ta-TS121-D; Table 1) in simplex (1 guide RNA; see FIG. 5) and multiplex (4 guide RNAs; FIG. 6). At all four target sites, A to G conversions ranging from 0.5% to 10% within an editing window of A08 to A11 were observed for v9h, v11h, and v12h (see FIGS. 5 and 6). These findings show that including the (i) truncated tRNA and the two mature direct repeats 5′- and 3′- of the sequence encoding the spacer RNA into the guide RNA architecture and (ii) the D156R mutant of dLbCas12a and (iii) the TadA9 domain into the LbCas12a-ABE architecture leads to increased editing efficiency.

TABLE 1
Target sites used to evaluate ABE activity at
endogenous targets in wheat and maize.

| | | SEQ ID |
|---|---|---|
| Name | Sequence | NO |
| Ta-TS60-A | TCTCCTACAAAGCTAGAGTATCA | 79 |
| Ta-TS60-B | TCTCCTACAAAGCTAGAGTAACA | 80 |
| Ta-TS60-D | TCTCCTACAAAGCTAGAGTAAGG | 81 |
| Ta-TS105-B | GTCTTACAAGAGGAAAGGTGGGG | 82 |
| Ta-TS112-A | AAGATAAATATGAGTCAGACCGT | 83 |
| Ta-TS112-B | AAGATAAATACGAGTCAGACGGT | 84 |
| Ta-TS112-D | AAGATAAATATGAGTCAGACGGT | 85 |
| Ta-TS121-D | TCTCCCTAATATTGCTCGTCTTT | 86 |
| Zm-TS1 | ACACGGGAAATTACAGCAGGAGA | 87 |
| Zm-TS3 | GGAAGGCGCAGATCGAGTCCGCG | 88 |
| Zm-TS4 | TCGTACGTACGTACCATGCATGC | 89 |
| Zm-TS8 | CATAGCACTAGCACCTGCTTTTG | 90 |

To determine whether the ABE configurations showing higher ABE activity in wheat protoplasts also lead to efficient adenine base editing in wheat plants the activity on the two most efficient wheat genomic target sites (Ta-TS60-A and Ta-TS112-A) was tested in stably transformed wheat plants.

Wheat transformation was performed according to Example 7: Immature embryos, 2-3 mm size, were isolated from sterilized ears of wheat cv. Fielder and bombarded using the PDS-1000/He particle delivery system (Bio-Rad) essentially as described by Sparks and Jones (2014; Cereal Genomics: Methods in Molecular biology, vol. 1099, Chapter 17) using the following particle bombardment parameters: diameter gold particles, 0.6 μm; target distance, 6 cm; bombardment pressure, 7.584 k Pa; gap distance, 8-10 mm; microcarrier flight distance, 10 mm; vacuum within the bombardment chamber, 27.5″ Hg. For each shot approximately 150 μg of gold particles carrying 570 ng of plasmid DNA were delivered. The applied plasmid DNA was a mixture of the vectors pCG392 (ABE v9: SEQ ID NO: 62) or pCG434 (ABE v12: SEQ ID NO: 63) (ABE), pCG406 (crRNA h: SEQ ID NO: 67) and pCG408 (crRNA h: SEQ ID NO: 68) (gRNAs) and pBAY02032 (Selectable Marker (SM)). The vector pBAY02032 contains an eGFP-BAR fusion gene under control of the 35S promoter. The further culture of the bombarded immature embryos was essentially conducted as described by Ishida et al. (2015; Agrobacterium Protocols: Volume 1, Methods in Molecular Biology, vol. 1223, Chapter 15, 189-198). Bombarded immature embryos were transferred to non-selective WLS callus induction medium for about one week, then moved to WLS with 5 mg/L phosphinothricin (PPT) for a 1st selection round of about 3 weeks followed by a 2nd selection round on WLS with 10 mg/L PPT for another 3 weeks. PPT resistant calli were selected and transferred to shoot regeneration medium with 5 mg/L PPT.

In 2 experiments a total of 689 and 756 immature embryos were bombarded with the mixture of pCG392 base editor, 2 gRNA and SM plasmid DNAs and phosphinotricin (PPT) tolerant shoot regenerating lines were obtained from in total 140 and 227 embryos. In 2 experiments, a total of 776 and 766 immature embryos were bombarded with the mixture of plasmid DNAs with pCG434 base editor and PPT tolerant shoot regenerating lines were obtained from in total 223 and 180 embryos. All plants developed from one immature embryo were treated as a pool. Genomic DNA was extracted from pooled leaf samples for ddPCR analysis. ddPCR assays were designed using Primer3Plus software with modified settings compatible with the applied master mix. To avoid loss of binding sites, primers and reference probe were designed away from the cut site. PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases. Drop-off probes were designed to lose their binding site when one or more base substitutions are introduced within the base editing activity window. The sequences of the probes and primers are shown in Table 2.

TABLE 2
Primers and probes used for the ddPCR drop-off assay to determine base
editing levels in transgenic wheat plants.

| | | | SEQ ID |
|---|---|---|---|
| Target site | Primer/probe type | Sequence | NO |
| Ta-TS60-A | Forward Primer | ACGACACTAAAGATGTGC | 91 |
| | Reverse Primer | GGGTAGAAAGAATGGTAAGT | 92 |
| | Reference probe (FAM) | ACATGCCTCTCCCTTCCAGTAG | 93 |
| | BE probe (HEX) | CTCTCCTACAAAGCTAGAGT | 94 |
| Ta-TS112-A | Forward Primer | TCCACTGACCAACCAAG | 95 |
| | Reverse Primer | GGTAGACTGAAAGTACCAAGA | 96 |
| | Reference probe (FAM) | TGCAAGGTACAGCTGCAGC | 97 |
| | BE probe (HEX) | AGATAAATATGAGTCAGACCGT | 98 |

20×ddPCR mixes were composed of 18 μM forward and 18 μM reverse primers, 5 μM reference probe and 5 μM drop-off probe. The following reagents were mixed in a 96-well plate to make a 25-μl reaction: 11 μl of ddPCR Supermix for Probes (no dUTP), 1.1 μl of 10× assay mix (BioRad Laboratories, Hercules, CA, USA), 100-250 ng of genomic DNA in water, and water up to 22 μl. Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories, Hercules, CA, USA). Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min. After PCR, the droplets were analyzed using a QX100 Droplet Reader (BioRad Laboratories, Hercules, CA, USA) in ‘absolute quantification’ mode. The ddPCR drop-off levels are a measure for the level of base editing in the plant. Table 3 summarizes the observed editing frequencies. For Ta-TS60-A, between 17 and 38% of the pools have more than 10% edits and between 3 and 10% of the pools have more than 50% edits. Target site Ta-TS112-A shows higher levels of editing, with 35 to 57% of the pools having more than 10% edits and 9 to 27% of the pools having more than 50% edits. This shows that in several of the pools efficient adenine base editing has happened.

TABLE 3
Editing frequencies at two wheat target sites of base editor constructs pCG392
and pCG434 in pools of transformed wheat shoots based on ddPCR drop-off levels.

| | | | | # pools | | | % pools | | |
|---|---|---|---|---|---|---|---|---|---|
| | ABE | # pools | Target | 50-100% | 10-50% | 0-10% | 50-100% | 10-50% | 0-10% |
| Experiment | vector | analyzed | site | drop-off | drop-off | drop off | drop-off | drop-off | drop off |
| TMTA0949 | pCG392 | 140 | TS60-A | 8 | 26 | 106 | 5.7 | 18.6 | 75.7 |
| | | | TS112-A | 27 | 39 | 74 | 19.3 | 27.9 | 52.9 |
| TMTA0950 | pCG392 | 227 | TS60-A | 7 | 31 | 189 | 3.1 | 13.7 | 83.3 |
| | | | TS112-A | 21 | 57 | 147 | 9.3 | 25.3 | 65.3 |
| TMTA0951 | pCG434 | 223 | TS60-A | 23 | 62 | 138 | 10.3 | 27.8 | 61.9 |
| | | | TS112-A | 61 | 67 | 95 | 27.4 | 30.0 | 42.6 |
| TMTA0952 | pCG434 | 180 | TS60-A | 15 | 51 | 114 | 8.3 | 28.3 | 63.3 |
| | | | TS112-A | 25 | 62 | 93 | 13.9 | 34.4 | 51.7 |

For nearly all shoots that showed base editing, the drop-off level was around 50% or 100%.

Individual T0 wheat plants were analyzed by NGS to determine editing frequencies and alleles at the on- and off-target loci. DNA was isolated using the Edwards method (Edwards et al, 1991, Nucleic Acids Research 19, 1349). Amplicons were obtained by PCR using Q5 Polymerase (Invitrogen) and were pooled and purified using PCR Purification Kit (Zymo Research Co., D4013). Paired end sequencing was performed (Eurofins NGSelect amplicons: 5M reads 2×150 bp) and base editing was calculated using CRISPResso2 (Clement et al., 2019 Nature Biotechnology 37, 224-226).

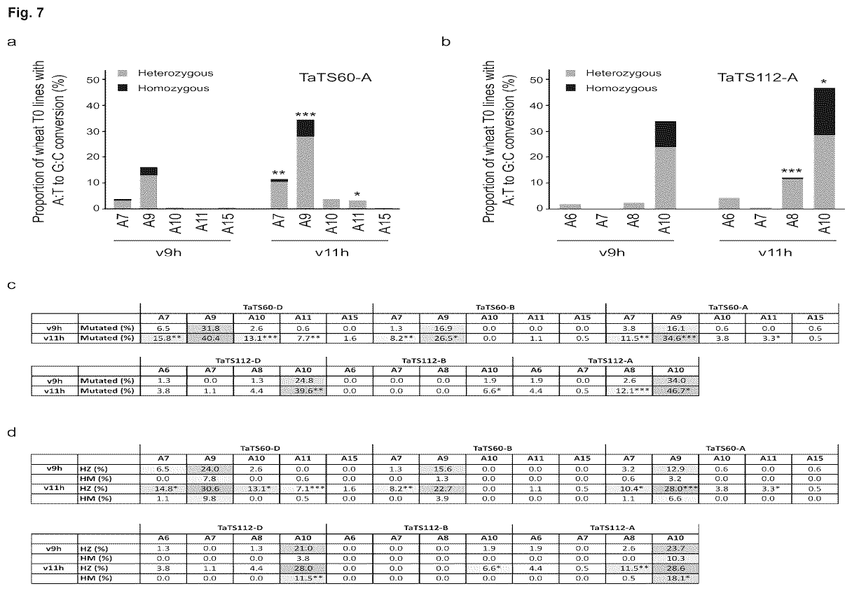

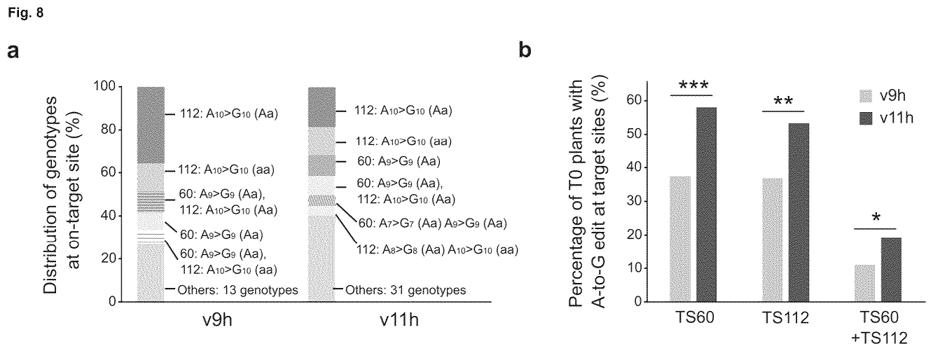

The proportion of wheat plants carrying AT to G:C conversions at position A7 and A9 for TaTs60-A and at position A8 and A10 for TaTS112-A ranged from 2.6 to 34% for v9h and from 11.5 to 46.7% for v11h (n>153 for each BE architectures; FIG. 7a-c). Consistent with results from protoplasts, v11h efficiency was significantly higher than v9h at these positions (1.4-4.7 fold increase, p<0.05 z-score test for two proportions; FIG. 7a-c). Base editing was also observed at secondary positions (A10, A11 and A15 for TaTS60-A and A6, A7 for TaTS112-A) and no indels were detected in any line. Altogether, v9h and v11h activity at TS60-A and TS112-A created a panel of 18 and 37 unique genotypes, respectively (FIG. 8a).

In line with previous protoplast results, Ta-TS112 was more active than Ta-TS60; 34-47% of the independent events were edited at TS112-A and 16-35% were edited at TS60-A (FIG. 7).

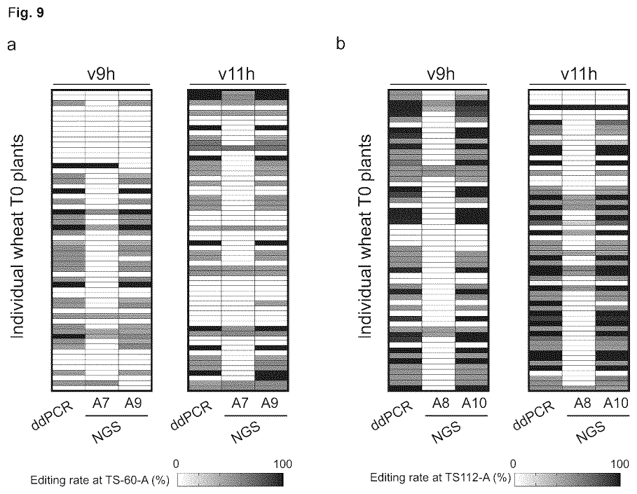

For a subset of the individual T0 lines (n>54), we compared editing rates obtained by ddPCR and NGS methods. For both v9h and v11h at TaTS60-A and TaTS112-A, we observe a strong correlation between editing rates obtained from the two methods (FIG. 9a-b) demonstrating that our ddPCR assay reliably predicts AT to G:C mutation levels.

We also analyzed off-target edits on homologous subgenomes by NGS (FIG. 7c-d, target sites TaTS60-D, TaTS60-B, TaTS112-D, and TaTS112-B) and observed A:T to G:C conversion consistent with previous results in protoplasts. The majority of the mutated lines were scored as heterozygous with 13 and 29% of the lines containing heterozygous on-target edits for the v11h architecture for TS60 and TS112, respectively (FIG. 7d). Homozygous lines were also recovered, with 3-18% for the primary target bases. Around 30% of the plants transformed with v9h carried at least one base edit in at least one of the homeologs at TS60 or TS112 and averaged 50% for v11h (FIG. 8b; p<0.01; z-score test for two proportions). Furthermore, double mutants for TS60 and TS112 were generated at a rate of 11% for v9h and 19% for v11h (FIG. 8b). These results show that the v9h and v11h Cas12a-ABEs can efficiently induce base editing in stable wheat lines without inducing indels. In the specific architecture as provided, and contrary to the literature (see Li et al., 2022, supra) our ABEs thus showed robust base editing efficiency without serious off-target effects.

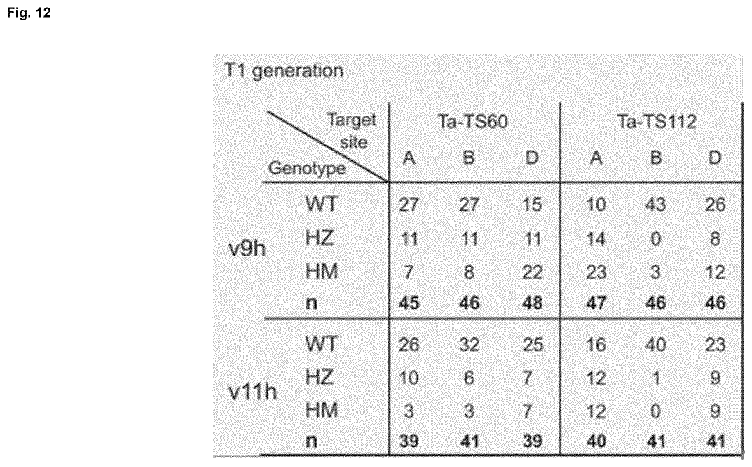

As confirmation, a subset of the T1 progeny was genotyped to confirm that Cas12a-ABEs could be used to generate stably transmitted alleles. To rule out continuous activity of the Cas12-ABEs in the T1s, we first screened for the absence of a functional Cas12a-ABE transgene in 20 v9h and 20 v11h lines. We selected 12 lines for both architectures and genotyped TS60 and TS112 sites via NGS from three or four transgene-free plants each (94 plants in total). Ten out of twelve and twelve out of twelve of the v9h and v11h lines contained edits in T1, respectively. Mutations were either heterozygous or homozygous A-to-G edits at all target sites and subgenomes for both architectures and no indels were detected (FIG. 12). Together, these results show that both Cas12a-ABEs can be used to efficiently introduce inheritable multiplex base edits in wheat without indels.

### Example 10.2: Cas12a-ABE Activity at Endogenous Target Sites in Maize (Zea mays)

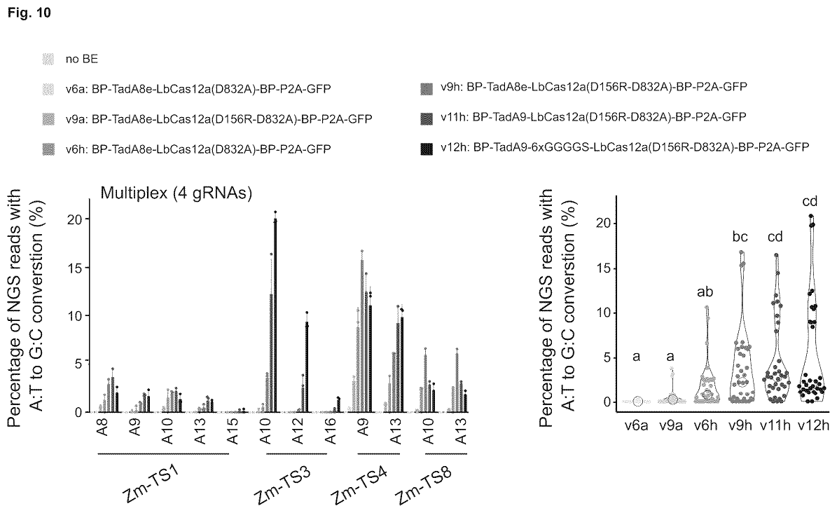

Similar experiments as described above for wheat were conducted for maize protoplasts (see FIG. 10). Base editing activity of optimized LbCas12a-ABE was determined by measuring A to G substitutions at endogenous maize genome sites. Six LbCas12a-ABE and crRNAs architectures were selected (numbers and letters according to nomenclature of FIGS. 1a and b) and their respective ABE activity was determined at four sites (Zm-TS1, Zm-TS3, Zm-TS4, and Zm-TS8; Table 1) in multiplex (4 guide RNAs; FIG. 10). Analogously to the results determined for wheat, v9h, v11h, and v12h performed best with editing efficiencies ranging from 0.5 to 20% (see FIG. 10).

Next, the activity of three Cas12a-ABEs architectures (v9h, v11h and v12h) was evaluated and compared at four endogenous sites in multiplex in maize T0 plants. Vectors containing a WUS-BBM cassette and an array of four crRNAs targeting Zm-TS1, Zm-TS3, Zm-TS4 and Zm-TS8 were constructed and transformed in Agrobacterium tumefaciens strain EHA105 according to Aesaert et al., 2022 (Aesaert, S., Impens, L., Coussens, G., Van Lerberge, E., Vanderhaeghen, R., Desmet, L., Vanhevel, Y., Bossuyt, S., Wambua, A. N., Van Lijsebettens, M., Inze, D., De Keyser, E., Jacobs, T. B., Karimi, M., Pauwels, L, 2022. Optimized Transformation and Gene Editing of the B104 Public Maize Inbred by Improved Tissue Culture and Use of Morphogenic Regulators. Frontiers in Plant Science 13.).

The four target sites Zm-TS1, Zm-TS3, Zm-TS4 and Zm-TS8 as well as the molecular tools used are shown in Table 4 below, SEQ ID NOs: 103 to 105 show specific maize targeting ABE constructs as designed, produced and used to target Zm-TS1, Zm-TS3, Zm-TS4 and Zm-TS8.

Maize transformation was conducted from immature maize embryos similarly to Aesaert et al., 2022. Leaf samples from individual regenerating shoots were harvested for DNA isolation and genotyping similarly to wheat analysis.

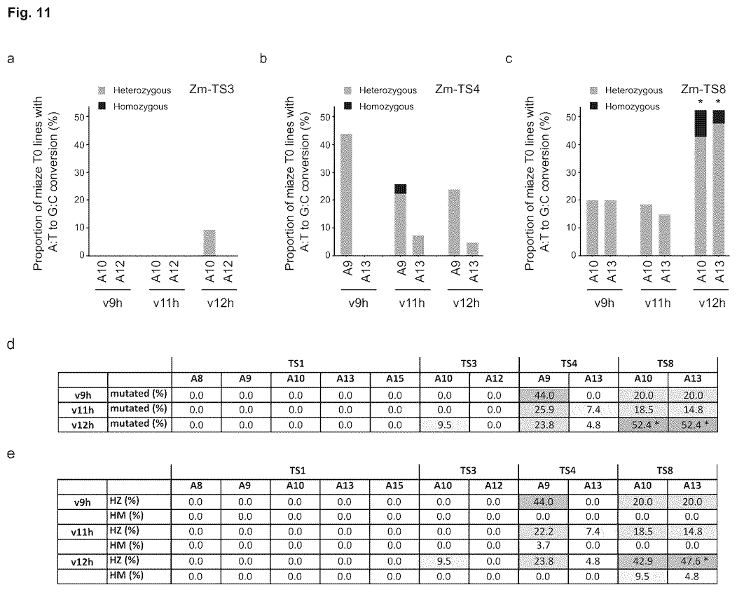

For v9h and v11h, we detected AT to G:C conversion at two out of four sites (Zm-TS4-, Zm-TS8) with up to 44% of the plants edited (FIG. 11b,d). In contrast, three out of four sites were edited for v12h (Zm-TS3, Zm-TS4 and Zm-TS8) with up to 52.4% of the plants edited (FIG. 11c,d). Consistently, v12h also showed a significantly higher activity at Zm-S8 (FIG. 11c-e; p<0.05: z-score test for two proportions). Most of the plants were shown to be heterozygous for the mutation, but also homozygous mutations for v11h and v12h were observed (cf. FIG. 11b, c, e). Similarly to the above results in wheat stable plants, none of the regenerated T0 plants showed indels. Analysis of progeny plants from one v9h, two v11h and one v12h maize lines showed that both the transgene insert and A-to-G edits were inherited to the next generation.

These results confirm that the optimized Cas12-ABEs can stably introduce A:T to G:C mutations at endogenous sites of another monocotyledon species and, therefore the optimized ABEs can be broadly and successfully applied in various plant species.

TABLE 4
Target sites used in Example 10.2

| | | | Target site | F primer | R primer |
|---|---|---|---|---|---|
| Target Site | Gene name | Gene ID | sequence | (5′-3#) | (5′-3′) |
| Zm-TS1 | FASCIATED | Zm00007a00002650 | SEQ ID | SEQ ID | SEQ ID |
| | EAR 3 (FEA3) | | NO: 87 | NO: 106 | NO: 107 |
| Zm-TS3 | SMALL KERNEL 1 | Zm00007a00012736 | SEQ ID | SEQ ID | SEQ ID |
| | (SNK1) | | NO: 88 | NO: 108 | NO: 109 |
| Zm-TS4 | LIGULELESS 2 | Zm00007a00014906 | SEQ ID | SEQ ID | SEQ ID |
| | (LG2) | | NO: 89 | NO: 110 | NO: 111 |
| Zm-TS8 | DWARF4 | Zm00007a00019935 | SEQ ID | SEQ ID | SEQ ID |
| | (DWF4) | | NO: 90 | NO: 112 | NO: 113 |

### Example 11: LbCas12a-ABE Activity in Soybean and Oilseed Rape

To test the activity of LbCas12a-ABE in dicot plants, additional experiments using oilseed rape (Brassica napus) and soybean (Glycine max) protoplasts were performed. Oilseed rape protoplasts were isolated from the leaves of 4- to 7-week-old aseptically grown plants. Healthy leaves were cut into fine strips with a sharp razor blade. The strips were infiltrated with cell wall-dissolving enzyme solution (1.5% cellulase R10 and 0.75% macerozyme R10 in 10 mM KCl and 0.6 M mannitol, pH 7.5) and incubated overnight in the dark with gentle shaking (40 rpm) at 24° C. After enzymatic digestion, the released protoplasts were collected by filtering the mixture through 40-μm nylon meshes and resuspended in W5 solution. The resuspended protoplasts were kept on ice and allowed to settle by gravity, after which the cell pellet was resuspended in MMG. For transformation, 200 μl of cells (2.5×105) were mixed with 20 μg plasmid DNA and 220 μl of freshly prepared polyethylene glycol (PEG) solution. The mixture was incubated for 15-20 min in the dark. After removing the PEG solution, the protoplasts were resuspended in 2 ml of W5 solution and incubated at 24° C. Soybean protoplasts were isolated from the unifoliate leaves of 6-day-old seedlings and transfected essentially as described for oilseed rape. After removing the PEG solution, the protoplasts were resuspended in 2 ml of WI solution.

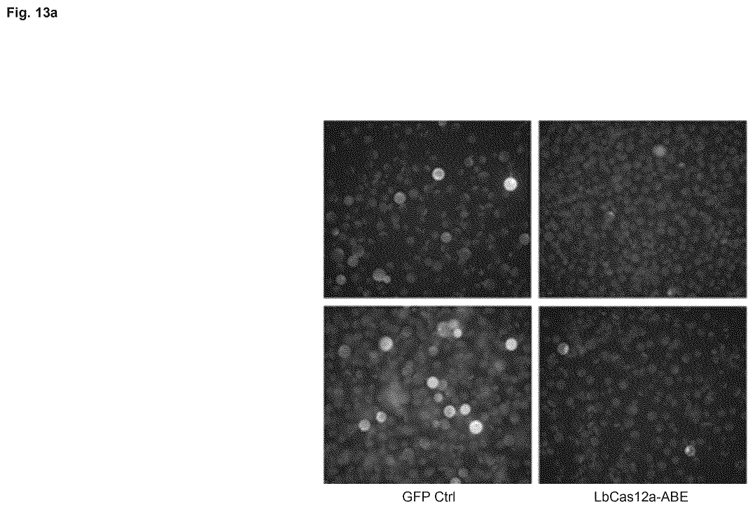

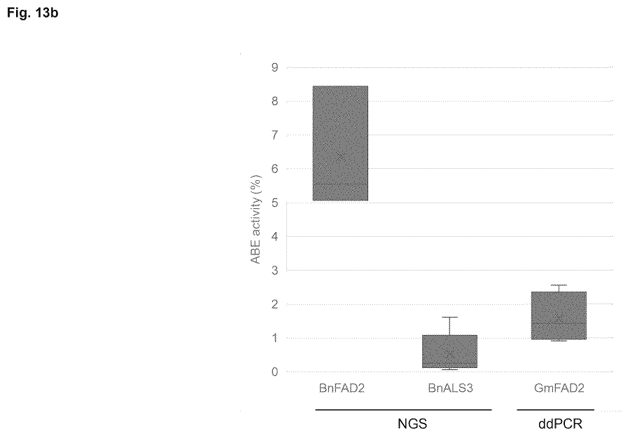

Cas12a-ABE activity was first evaluated using a GFP reporter system similar to that described in wheat. To this end, oilseed rape protoplasts were co-transfected with 3 vectors: (1) a vector encoding a mutated GFP gene containing an early stop codon (SEQ ID NO 118) (2) a Cas12a-ABE expression construct comprising TadA9 as an adenosine deaminase domain and a hexa-GGGGS linker connecting TadA9 to a dLbCas12a (D156R) module located 3′ of TadA9 (see construct 12 in FIG. 1a; SEQ ID NO 119) and (3) a vector encoding a gRNA targeting the dGFP reporter and containing two mature direct repeats 5′ and 3′ of the spacer (SEQ ID NO 120). The Cas12a-ABE vector included the Arabidopsis ubiquitin10 promoter for constitutive expression, while expression of the gRNA was driven by the polymerase III-type promoter of the Arabidopsis U6 snRNA gene. Editing of the TAG stop codon into the original CAG codon restores the GFP coding sequence and results in GFP fluorescence. As a positive control, protoplasts were transfected with a construct harboring wild-type GFP behind a strong cauliflower mosaic virus (CaMV) 35S promoter. As a negative control the Cas12a-ABE fusion protein was tested without the gRNA. Fluorescence imaging at 2 days post transfection revealed approximately 35% GFP-fluorescent cells in the positive control and 4.2% with the Cas12a-ABE (see FIG. 13a). Importantly, no GFP-positive cells could be observed in the absence of the gRNA (data not shown).

To confirm the Cas12a-ABE activity at endogenous target sites, the TadA9>(GGGSS)6×>dLbCas12a-D156R expression construct was co-transfected into oilseed rape or soybean protoplasts along with an expression construct for a Cas12a gRNA targeting the BnFAD2 (SEQ ID NO 121), BnALS3 (SEQ ID NO 122) or GmFAD2 (SEQ ID NO 123) genes, respectively. Transfected oilseed rape protoplasts were cultured in alginate and editing efficiencies were determined at 14 days post transfection by deep amplicon sequencing. Conversely, soybean protoplasts were incubated in WI solution for 72 hours and analyzed via droplet digital PCR. As shown in FIG. 13b, expression of Cas12a-ABE resulted in relatively high editing efficiencies at the FAD2 target site, with up to 8.4% of the sequence reads showing A-to-G substitutions and less than 0.025% showing indel formation. Lower but significant levels of base editing were observed when targeting the BnALS3 or GmFAD2 genes (average of 0.52% and 1.58%, respectively). Together with the data in wheat and maize, these results show that the TadA9-containing ABE is active in both monocot and dicot plants.

1. An adenine base editor (ABE) comprising, in sequential order, the following structural elements:
a.) at least one N-terminal NLS sequence;
b.) a TadA9 adenosine deaminase domain, or a functional variant thereof;
c.) at least one linker domain;
d.) a dCas12a, or a functional fragment thereof, or a nCas12a, or a functional fragment thereof, wherein the dCas12a or the nCas12a, or a functional fragment thereof, comprises at least one or more mutations, wherein the at least one or more mutations confer increased activity and/or enhanced temperature tolerance, wherein one of the at least one or more mutations corresponds to a mutation in a dCas12a ortholog or homolog at a position homologous to D156 of SEQ ID NOs: 14, 15, or 16, E174 of SEQ ID NOs: 17, 18, or 19, and E184 of SEQ ID NOs: 20 to 28, respectively, the at least one mutation conferring increased activity and/or enhanced temperature tolerance, particularly wherein the at least one mutation in the dCas12a ortholog or homolog corresponds to a D to R, an E to R, or a K to D/E mutation at the homologous position of SEQ ID NOs: 14 to 43 as reference, respectively; and
e.) at least one C-terminal NLS sequence;
wherein the at least one N-terminal and the at least one C-terminal NLS sequence can be the same or different.

2. An adenine base editor (ABE) comprising, in sequential order, the following structural elements:
a.) at least one N-terminal NLS sequence;
b.) an adenosine deaminase domain being selected from a TadA8, or a TadA9 domain, or a functional variant thereof;
c.) at least one linker domain, wherein the at least one linker comprises or consists of a hexa-GGGGS linker according to SEQ ID NO: 51;
d.) a dCas12a, or a functional fragment thereof, or a nCas12a, or a functional fragment thereof; and
e.) at least one C-terminal NLS sequence;
wherein the at least one N-terminal and the at least one C-terminal NLS sequence can be the same or different.

3. The adenine base editor according to claim 1, wherein the dCas12a or the nCas12a, or the functional fragment thereof, comprises at least one or more additional mutations as defined in claim 1, wherein one of the at least one or more additional mutations conferring increased activity and/or enhanced temperature tolerance corresponds to a mutation in a dCas12a ortholog or homolog at a position homologous to position D156 of SEQ ID NO: 14, 15, or 16, or to position E174 of SEQ ID NO: 17, 18, or 19, or to position E184 of SEQ ID NO: 20, 21, 22, 23, 24, 25, 26, 27, or 28, or to a homologous position within a Cas12a ortholog or homolog; or wherein one of the at least one or more additional mutations conferring increased activity and/or temperature tolerance corresponds to D156R in comparison to SEQ ID NO: 14, 15, or 16 as reference sequences, or at an homologous position within a Cas12a ortholog or homolog, or wherein one of the at least one or more additional mutations conferring increased activity and/or temperature tolerance corresponds to E174R in comparison to SEQ ID NO: 17, 18, or 19 as reference sequences, or at an homologous position within a Cas12a ortholog or homolog, or wherein one of the at least one or more additional mutations conferring increased activity and/or temperature tolerance corresponds to E184R in comparison to SEQ ID NO: 20, 21, 22, 23, 24, 25, 26, 27, or 28 as reference sequences, or at an homologous position within a Cas12a ortholog or homolog; or
wherein the at least one or more additional mutations correspond to (i) D156R and D832A or (ii) D156R and E925A or (iii) D156R and D832A and E925A in comparison to SEQ ID NO: 1 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog, or wherein the at least one or more additional mutations correspond to (iv) E174R and D908A or (v) E174R and E993A or (vi) E174R and D908A and E993A in comparison to SEQ ID NO: 2 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog,
or wherein the at least one or more additional mutations correspond to (viii) E184R and D917A or (ix) E184R and E1006A or (x) E184R and D917A and E1006A in comparison to SEQ ID NO: 3 as a reference sequence or in comparison to a sequence having at least 75%, 76%, 77%, 78%, 79%, 80%, 81%, 82%; 83%, 84%, 85%, 86%, 87%, 88%, 89%, 90%, 91%, 92%, 93%, 94%, 95%, 96%, 97%, 98% or 99% sequence identity to the corresponding reference sequence, or at homologous positions within a Cas12a ortholog or homolog.

4. The adenine base editor according to claim 1, wherein the at least one N-terminal NLS sequence and/or the at least one C-terminal NLS sequence is/are selected from a triple SV40 NLS of SEQ ID NO: 52, a bipartite SV40 NLS of SEQ ID NO: 53, a SV40 NIS of SEQ ID NO: 54, a FNLS of SEQ ID NO: 55, or a nucNLS of SEQ ID NO: 56, or wherein the at least one N-terminal and the at least one C-terminal NLS sequence is at least one bipartite SV40 NLS of SEQ ID NO: 53, or a functional homolog thereof, or a sequence having at least 95%, 96%, 97%, 98% or at least 99% sequence identity to SEQ ID NO: 53.

5. The adenine base editor according to claim 2, wherein the adenosine deaminase domain is a TadA8e domain according to SEQ ID NO: 57, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 57, or wherein the adenosine deaminase domain is a TadA9 according to SEQ ID NO: 58, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 58.

6. A complex comprising an adenine base editor according to claim 1 and a guide RNA in a functionally associated form, or a nucleic acid molecule encoding the guide RNA, wherein the guide RNA is specific for the dCas12a or for the nCas12a as defined in claim 1, optionally wherein the guide RNA is expressed from a construct comprising a truncated tRNA at the 5′ end and at least one direct repeat structure 5′- and 3′- of the sequence of or encoding the spacer RNA.

7. The complex of claim 6, wherein the guide RNA is encoded by a scaffold architecture as provided with any one of SEQ ID NO: 59, 60, or 61, or a sequence having at least 90%, 91%, 92%, 93%, 94%, 95%, 96%, 97%, 98% or 99% sequence identity to at least one of the corresponding reference sequences of SEQ ID NO: 59, 60, or 61, respectively.

8. A nucleic acid molecule encoding the adenine base editor according to claim 1, and/or a nucleic acid molecule encoding a guide RNA in a functionally associated form, wherein the guide RNA is specific for the dCas12a or for the nCas12a as defined in claim L optionally wherein the guide RNA is expressed from a construct comprising a truncated tRNA at the 5′ end and at least one direct repeat structure 5′- and 3′- of the sequence of or encoding the spacer RNA.

9. An expression construct or a vector comprising a nucleic acid sequence according to the nucleic acid molecule of claim 8, wherein the nucleic acid sequence encoding the adenine base editor and/or the nucleic acid sequence encoding the guide RNA are present (i) on the same expression construct or vector, or (ii) wherein the nucleic acid sequence encoding the adenine base editor and/or the nucleic acid sequence encoding the guide RNA are present on at least two individual expression constructs or vectors, optionally wherein an expression construct or vector encoding a guide RNA is present and wherein the guide RNA is expressed from an RNA polymerase III promoter or an RNA polymerase II promoter.

10. A cell comprising an adenine base editor according to claim 1.

11. A method of adenine base editing of a target site in a genome of interest in at least one cell of a prokaryotic or eukaryotic organism, the method comprising the following steps:
(a) providing at least one adenine base editor or at least one complex according to claim 1, or a nucleic acid molecule or expression construct encoding the same, to the at least one cell;
(b) optionally: allowing functional expression and/or assembly of a complex into a functionally associated form;
(c) contacting the genome of interest of the at least one cell with at least one functionally associated form of a complex comprising at least one adenine base editor according to claim 1 to obtain at least one modified cell;
(d) optionally: selecting the at least one modified cells; and
(e) obtaining at least one cell containing at least one adenine base edit at the target site, wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes and further wherein the method excludes the treatment of a human or animal body by therapy, optionally, where the method comprises the following step:
(f) regenerating at least one population of edited cells, tissues, organs, materials or whole organisms from the at least one edited cell.

12. The method according to claim 11, wherein the at least one cell is from a plant, algae, yeast or fungus organism.

13. An edited cell, or a tissue, organ, material or whole organism obtained by or obtainable by a method according to claim 11.

14. A kit comprising
(a) the adenine base editor according to claim 1,
and comprising
(b) a container containing reaction components including buffers
and optionally comprising
(c) instructions for use.

15. (canceled)

16. The adenine base editor according to claim 1, wherein the at least one linker comprises or consists of a hexa-GGGGS linker according to SEQ ID NO: 51.

17. The adenine base editor according to claim 2, wherein the adenosine deaminase domain is a TadA8e, or a functional variant thereof.

18. The expression construct or a vector of claim 9, wherein the promoter is U3, U6, H1, or a ubiquitin promoter.

19. The method according to claim 11, wherein the at least one cell is a plant cell belonging to superfamily Viridiplantae, or is a plant cell from fodder or forage legumes, ornamental plants, food crops, trees or shrubs.

20. The adenine base editor according to claim 1, wherein the adenosine deaminase domain is a TadA9 according to SEQ ID NO: 58, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to SEQ ID NO: 58.


SEQUENCE LISTING (123 sequences)

SEQ ID NO: 1 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
  source 1..1307: note = strain BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1300 aa, Francisella tularensis
  source 1..1300: note = locus WP_216370596.1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 4 - protein, 1300 aa, Francisella tularensis
  source 1..1300: note = subsp. novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 5 - protein, 1300 aa, Francisella tularensis
  source 1..1300: note = subspecies novicida; note = strain U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 6 - protein, 1262 aa, Porphyromonas crevioricanis
MPWIDLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYEPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 7 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNLFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPWVFLSSKKGMETYKPSAYILEGYKQNKHLKSSKDFDITFCHDLIDYFKNCIAIHPEWKNFGFEFSDTSTYEDISGFYREVELQGYKIDWTYISEKDIELLQEKGQLYLFQIYNKDFSKKSTGNDNLHTMYLKNLFSEENLKDIVLKLNGKAEIFFRKSSIKNPIIHKKGSILVNRTYEAEEKEQFGNIRIVRKNIPENIYQELYKYYNDKSDKELSDEAAKLKDIVGHHEATKDIVKDYRYTYDKYFLHMPITINFKANKTSFINDRILQYIATEKELHVIGIDRGERNLIYVSVIDTCGNIVEQKSFNIVNGYDYQIKLKQQEGARQTARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSNWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYL

SEQ ID NO: 8 - protein, 1227 aa, Methanomethylophilus alvi
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 9 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 10 - protein, 1206 aa, Butyrivibrio sp.
  source 1..1206: note = strain NC3005
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 11 - protein, 1298 aa, Thiomicrospira sp.
  source 1..1298: note = strain XS5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 12 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 13 - protein, 1222 aa, unidentified
  source 1..1222: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLLKRPAATKKAGQAKKKK

SEQ ID NO: 14 - protein, 1228 aa, synthetic construct
  REGION 1..1228: note = dead Cas12a variant dLbCas12a - D832A mutant protein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 15 - protein, 1228 aa, synthetic construct
  REGION 1..1228: note = dead Cas12a variant dLbCas12a - E925A mutant protein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 16 - protein, 1228 aa, synthetic construct
  REGION 1..1228: note = dead Cas12a variant dLbCas12a - D832A/E925A mutant protein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRRERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 17 - protein, 1307 aa, synthetic construct
  REGION 1..1307: note = dead Cas12a variant dASCas12a D908A mutant protein
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 18 - protein, 1307 aa, synthetic construct
  REGION 1..1307: note = dead Cas12a variant dASCas12a - E993A mutant protein
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 19 - protein, 1307 aa, synthetic construct
  REGION 1..1307: note = dead Cas12a variant dAsCas12a - D908A/E993A mutant protein
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1300 aa, synthetic construct
  REGION 1..1300: note = dead Cas12a variant dFtCas12a - D917A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 21 - protein, 1300 aa, synthetic construct
  REGION 1..1300: note = dead Cas12a variant dFtCas12a - E1006A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVIFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 22 - protein, 1300 aa, synthetic construct
  REGION 1..1300: note = dead Cas12a variant dFtCas12a - D917A/E1006A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVIFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 23 - protein, 1300 aa, synthetic construct
  REGION 1..1300: note = dead Cas12a variant dFnCas12a - D917A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 24 - protein, 1300 aa, synthetic construct
  REGION 1..1300: note = dead Cas12a variant dFnCas12a - E1006A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 25 - protein, 1300 aa, synthetic construct
  REGION 1..1300: note = dead Cas12a variant dFnCas12a - D917A/E1006A protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEHNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 26 - protein, 1300 aa, synthetic construct
  REGION 1..1300: note = dead Cas12a variant dFnCas12a (strain U112) - D917A mutantprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 27 - protein, 1300 aa, synthetic construct
  REGION 1..1300: note = dead Cas12a variant dFnCas12a (strain U112) - E1006A mutantprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 28 - protein, 1300 aa, synthetic construct
  REGION 1..1300: note = dead Cas12a variant dFnCas12a (strain U112) - D917A/E1006A mutantprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 29 - protein, 1228 aa, synthetic construct
  REGION 1..1228: note = thermotolerant dead Cas12a variant LbCas12a - D832A/D156R mutantprotein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 30 - protein, 1228 aa, synthetic construct
  REGION 1..1228: note = thermotolerant dead Cas12a variant LbCas12a - E925A/D156R mutantprotein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 31 - protein, 1228 aa, synthetic construct
  REGION 1..1228: note = thermotolerant dead Cas12a variant LbCas12a - D832A/E925A/D156Rmutant protein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 32 - protein, 1307 aa, synthetic construct
  REGION 1..1307: note = thermotolerant dead Cas12a variant AsCas12a - D908A/E174R
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 33 - protein, 1307 aa, synthetic construct
  REGION 1..1307: note = thermotolerant dead Cas12a variant AsCas12a - E993A/E174R
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 34 - protein, 1307 aa, synthetic construct
  REGION 1..1307: note = thermotolerant dead Cas12a variant AsCas12a - D908A/E993A/E174R
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 35 - protein, 1300 aa, synthetic construct
  REGION 1..1300: note = thermotolerant dead Cas12a variant dFtCas12a - D917A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 36 - protein, 1300 aa, synthetic construct
  REGION 1..1300: note = thermotolerant dead Cas12a variant dFtCas12a - E1006A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVIFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 37 - protein, 1300 aa, synthetic construct
  REGION 1..1300: note = thermotolerant dead Cas12a variant dFtCas12a - D917A/E1006A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVIFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 38 - protein, 1300 aa, synthetic construct
  REGION 1..1300: note = thermotolerant dead Cas12a variant dFnCas12a - D917A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 39 - protein, 1300 aa, synthetic construct
  REGION 1..1300: note = thermotolerant dead Cas12a variant dFnCas12a - E1006A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 40 - protein, 1300 aa, synthetic construct
  REGION 1..1300: note = thermotolerant dead Cas12a variant dFnCas12a - D917A/E1006A/E184Rprotein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEHNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 41 - protein, 1300 aa, synthetic construct
  REGION 1..1300: note = thermotolerant dead Cas12a variant dFnCas12a (strain U112) -D917A/E184R mutant protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 42 - protein, 1300 aa, synthetic construct
  REGION 1..1300: note = thermotolerant dead Cas12a variant dFnCas12a (strain U112) -E1006A/E184R mutant protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 43 - protein, 1300 aa, synthetic construct
  REGION 1..1300: note = thermotolerant dead Cas12a variant dFnCas12a (strain U112) -D917A/E1006A/E184R mutant protein
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHRNRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 44 - protein, 1228 aa, synthetic construct
  REGION 1..1228: note = LbCas12a RR mutant protein (G532R/K595R), recognizes TYCV PAMsites
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 45 - protein, 1228 aa, synthetic construct
  REGION 1..1228: note = LbCas12a RVR mutant protein (G532R/K538V/Y542R), recognizes TATVPAM sites
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 46 - protein, 1228 aa, synthetic construct
  REGION 1..1228: note = LbCas12a RVRR mutant protein (G532R/K538V/Y542R/K595R),recognizes TACV, CTCV, and CCCV PAM sites
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 47 - protein, 1228 aa, synthetic construct
  REGION 1..1228: note = enLbCas12a mutant protein (D156R/G532R/K538R), recognizes TTYN,VTTV, and TRTV PAM sites, plus the RVRR PAM sites
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 48 - protein, 32 aa, synthetic construct
  REGION 1..32: note = XTEN 32aa linker
SGGSSGGSSGSETPGTSESATPESSGGSSGGS

SEQ ID NO: 49 - protein, 48 aa, synthetic construct
  REGION 1..48: note = XTEN 48aa linker
SGSETPGTSESATPESSGGSSGGSSGSETPGTSESATPESSGGSSGGS

SEQ ID NO: 50 - protein, 5 aa, synthetic construct
  REGION 1..5: note = GGGGS linker
GGGGS

SEQ ID NO: 51 - protein, 30 aa, synthetic construct
  REGION 1..30: note = Hexa-GGGGS linker
GGGGSGGGGSGGGGSGGGGSGGGGSGGGGS

SEQ ID NO: 52 - protein, 29 aa, synthetic construct
  REGION 1..29: note = Triple SV40 nuclear localization signal sequence
PKKKRKVSGGSPKKKRKVSGGSPKKKRKV

SEQ ID NO: 53 - protein, 17 aa, synthetic construct
  REGION 1..17: note = Bipartite SV40 nuclear localization signal sequence
KRTADGSEFEPKKKRKV

SEQ ID NO: 54 - protein, 7 aa, synthetic construct
  REGION 1..7: note = SV40 nuclear localization signal sequence
PKKKRKV

SEQ ID NO: 55 - protein, 31 aa, synthetic construct
  REGION 1..31: note = Flag-tagged SV40 nuclear localization signal sequence
DYKDHDGDYKDHDIDYKDDDDKMAPKKKRKV

SEQ ID NO: 56 - protein, 16 aa, synthetic construct
  REGION 1..16: note = nucNLS
KRPAATKKAGQAKKKK

SEQ ID NO: 57 - protein, 164 aa, synthetic construct
  REGION 1..164: note = Transfer RNA adenine deaminase 8e sequence (TadA8e)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSS

SEQ ID NO: 58 - protein, 164 aa, synthetic construct
  REGION 1..164: note = Transfer RNA adenine deaminase 9 sequence (TadA9)
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSS

SEQ ID NO: 59 - DNA, 118 bp, synthetic construct
  misc_feature 1..118: note = Sequence for scaffold architecture for LbCas12a
  misc_feature 89: note = n is a, c, g, or t
  source 1..118: mol_type = other DNA
ccagtggtctagtggtagaatagtaccctgccacggtacagacccgggttcgattcccggctggaaataatttctactaagtgtagatntaatttctactaagtgtagattttttttt

SEQ ID NO: 60 - DNA, 116 bp, synthetic construct
  misc_feature 1..116: note = Sequence for scaffold architecture for AsCas12a
  misc_feature 88: note = n is a, c, g, or t
  source 1..116: mol_type = other DNA
ccagtggtctagtggtagaatagtaccctgccacggtacagacccgggttcgattcccggctggaaataatttctactcgtgtagatntaatttctactcgtgtagattttttttt

SEQ ID NO: 61 - DNA, 116 bp, synthetic construct
  misc_feature 1..116: note = Sequence for scaffold architecture for FnCas12a
  misc_feature 88: note = n is a, c, g, or t
  source 1..116: mol_type = other DNA
ccagtggtctagtggtagaatagtaccctgccacggtacagacccgggttcgattcccggctggaaataatttctactggtgtagatntaatttctactggtgtagattttttttt

SEQ ID NO: 62 - DNA, 11321 bp, synthetic construct
  misc_feature 1..11321: note = pCG392 : pZmUBI-BP-TadA8e-XTENlinker-LbCas12a(D156R-D832A)-BP-G7T
  source 1..11321: mol_type = other DNA
ggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtgcgactgtctagaatcgcgtatgcgagtgtatctagaatcgcgtatgcgagtgtatgagctcgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagacttccgactgagcctttcgttttatttgatgcctggcagttccctactctcgcgttaacgctagcatggatgttttcccagtcacgacgttgtaaaacgacggccagtcttaagctcgggcccacctccgatccattcggtccccagattagccttttcaatttcagaaagaatgctaacccacagatggttagagaggcttacgcagcacgtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacgagctaacagtattcagtcgactggtaccaacaggctccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtcaggttctggttcaggttcaggttctggatctggaagtggatctggatcagcggccgcatctgaattcaagcgtgaagagcaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaactgcctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtca

SEQ ID NO: 63 - DNA, 11321 bp, synthetic construct
  misc_feature 1..11321: note = pCG434 : pZmUBI-BP-TadA9-XTEN linker-LbCas12a(D156R-D832A)-BP-G7T
  source 1..11321: mol_type = other DNA
ggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtgcgactgtctagaatcgcgtatgcgagtgtatctagaatcgcgtatgcgagtgtatgagctcgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagacttccgactgagcctttcgttttatttgatgcctggcagttccctactctcgcgttaacgctagcatggatgttttcccagtcacgacgttgtaaaacgacggccagtcttaagctcgggcccacctccgatccattcggtccccagattagccttttcaatttcagaaagaatgctaacccacagatggttagagaggcttacgcagcacgtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacgagctaacagtattcagtcgactggtaccaacaggctccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtcaggttctggttcaggttcaggttctggatctggaagtggatctggatcagcggccgcatctgaattcaagcgtgaagagcaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaactgcctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtactcgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacgggtgttcaatgctcagaagaaggcccagagctccatcaactccggaggatctagcggaggctcctctggctctgagacacctggcacaagcgagagcgcaacacctgaaagcagcgggggcagcagcggggggtca

SEQ ID NO: 64 - DNA, 11315 bp, synthetic construct
  misc_feature 1..11315: note = pCG463: pZmUBI-BP-TadA8e-6xGGGGSlinker-LbCas12a(D15 6R-D832A)-BP-G7T
  source 1..11315: mol_type = other DNA
ggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtgcgactgtctagaatcgcgtatgcgagtgtatctagaatcgcgtatgcgagtgtatgagctcgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagacttccgactgagcctttcgttttatttgatgcctggcagttccctactctcgcgttaacgctagcatggatgttttcccagtcacgacgttgtaaaacgacggccagtcttaagctcgggcccacctccgatccattcggtccccagattagccttttcaatttcagaaagaatgctaacccacagatggttagagaggcttacgcagcacgtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacgagctaacagtattcagtcgactggtaccaacaggctccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtcaggttctggttcaggttcaggttctggatctggaagtggatctggatcagcggccgcatctgaattcaagcgtgaagagcaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaactgcctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaacggtggcggaggttctggcggtggaggatccggaggtggcgggtccggtggtggcggatcaggtggaggaggttcgggaggcggaggtagt

SEQ ID NO: 65 - DNA, 11315 bp, synthetic construct
  misc_feature 1..11315: note = pCG466: pZmUBI-BP-TadA9-6xGGGGSlinker-LbCas12a(D156R-D832A)-BP-G7T
  source 1..11315: mol_type = other DNA
ggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtgcgactgtctagaatcgcgtatgcgagtgtatctagaatcgcgtatgcgagtgtatgagctcgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagacttccgactgagcctttcgttttatttgatgcctggcagttccctactctcgcgttaacgctagcatggatgttttcccagtcacgacgttgtaaaacgacggccagtcttaagctcgggcccacctccgatccattcggtccccagattagccttttcaatttcagaaagaatgctaacccacagatggttagagaggcttacgcagcacgtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacgagctaacagtattcagtcgactggtaccaacaggctccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtcaggttctggttcaggttcaggttctggatctggaagtggatctggatcagcggccgcatctgaattcaagcgtgaagagcaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaactgcctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtactcgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacgggtgttcaatgctcagaagaaggcccagagctccatcaacggtggcggaggttctggcggtggaggatccggaggtggcgggtccggtggtggcggatcaggtggaggaggttcgggaggcggaggtagt

SEQ ID NO: 66 - DNA, 3349 bp, synthetic construct
  misc_feature 1..3349: note = pTaU3-tRNA-matureDR- spacer-matureDR-polyT
  misc_feature 625..647: note = n is a, c, g, or t
  source 1..3349: mol_type = other DNA
ggtctcaacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatnnnnnnnnnnnnnnnnnnnnnnntaatttctactaagtgtagatttttttaacatgagaccgaattctcgccctatagtgagtcgtattacctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgaggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgagctatttaggtgacactatagaagtgaagctt

SEQ ID NO: 67 - DNA, 3349 bp, synthetic construct
  misc_feature 1..3349: note = pCG406: pTaU3-tRNA-matureDR-TS60A spacer-matureDR-polyT
  source 1..3349: mol_type = other DNA
ggtctcaacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagattctcctacaaagctagagtatcataatttctactaagtgtagatttttttaacatgagaccgaattctcgccctatagtgagtcgtattacctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgaggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgagctatttaggtgacactatagaagtgaagctt

SEQ ID NO: 68 - DNA, 3349 bp, synthetic construct
  misc_feature 1..3349: note = pCG408: pTaU3-tRNA-matureDR-TS112A gRNA-matureDR-polyT
  source 1..3349: mol_type = other DNA
ggtctcaacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagataagataaatatgagtcagaccgttaatttctactaagtgtagatttttttaacatgagaccgaattctcgccctatagtgagtcgtattacctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgaggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgagctatttaggtgacactatagaagtgaagctt

SEQ ID NO: 69 - DNA, 525 bp, synthetic construct
  misc_feature 1..525: note = Promoter
  source 1..525: mol_type = other DNA
catgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagca

SEQ ID NO: 70 - DNA, 858 bp, synthetic construct
  misc_feature 1..858: note = Promoter
  source 1..858: mol_type = other DNA
gaattcatcctcacgttcaacaccacatagggggcatacctcagtatcctcgaggtgccgtgaagccttgttggcccaagtagcaagtgaatttgtgacaacccttcatgcatatacggtagttttatttattttactctctacattgttctcatgatattcatccatgcagcctctctttcctggggaaaattcagttgatcaactggttgagattatcaaggtgaaatcttgtagtatgtacgacatttggaactgctttctcctgcgacatgcattctttacaatgacatgttcagccatagtactccaagaagggaagagaccaagtgcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagccctaccaacctcacgggccagcaattacgagtccttctaaaacgtcccgccgagggcgcgtggccgtgctgtgcagcagcacgtctaacattagtcccacctcgccagtttacagggagcagaaccagcttataagcggaggcgcggcaccaagaagca

SEQ ID NO: 71 - DNA, 363 bp, synthetic construct
  misc_feature 1..363: note = Promoter
  source 1..363: mol_type = other DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttg

SEQ ID NO: 72 - DNA, 415 bp, synthetic construct
  misc_feature 1..415: note = Promoter
  source 1..415: mol_type = other DNA
actctgcgaggacttctcttgtgaaaagctcgaaataaatctacatacacgtttatgttaaaatgtgagaagaatgtagaatgactagtataaaaacggagtcaaataaccagcgttctcccccacggtgaaaaaagaacctatcacatcggtagaccatttttttataggcaaatcatatcggcagactaacaggaaggctcactattccgtactagcacgatggcccattacggtcttccactattttgggccacaccgagagatccagcctactcgtagcccaccgcacgttccgacggcgcttccgtctggacaagcaacggggggagcagaaggctcgtttgtttagtcccacatcggctagcgaaagggagaacgaccagtttataagccacgcgctgcacacagactc

SEQ ID NO: 73 - DNA, 639 bp, synthetic construct
  misc_feature 1..639: note = Promoter
  source 1..639: mol_type = other DNA
ctcacagagtgcccctcatttatgtcaaactatacacttatcatgtctgacatgactttgcagttaatttttttgaacggtgtcatgctttgcacaattgtcctccatctataatgttttacaagaaactatttaatactgtattaacctttgcagtaggcatacttggtaccataattatctacatgctgtcatgtggagctcgtggtacatacagtagtttttttgttactaaatacggagtacattcgtatatgatatgttccctaattagttgcatcacttttttgtcctcattgctgctctactctaaacttatgttccagacaagcctacaactgagtcagcctgtcagagttctactgtgtttggatagatgttctagtgcaactttcttcttgtatgtttagttaatcataaggtctcattttcttactacctccgggacagatgtttctcagatgggccgggctctctctgcccagcaggcagcagcagcccgacctcatgggccagcacccaccacgagttccagctagggggtgttgctgcctaacactaacactaacattagtcccacctcgccagtttgcagggatcggaaccagcttataagctgagctgaggaagaaggtagca

SEQ ID NO: 74 - DNA, 79 bp, synthetic construct
  misc_feature 1..79: note = Promoter
  source 1..79: mol_type = other DNA
ggagtgatcaaaagtcccacatcgatcaggtgatatatagcagcttagtttatataatgatagagtcgacatagcgatt

SEQ ID NO: 75 - DNA, 446 bp, synthetic construct
  misc_feature 1..446: note = Promoter
  source 1..446: mol_type = other DNA
ttcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattg

SEQ ID NO: 76 - DNA, 306 bp, synthetic construct
  misc_feature 1..306: note = Promoter
  source 1..306: mol_type = other DNA
aaaataaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggattg

SEQ ID NO: 77 - DNA, 353 bp, synthetic construct
  misc_feature 1..353: note = Promoter
  source 1..353: mol_type = other DNA
atgcctatcttatatgatcaatgaggcatttaattgggtgcatatgatggtgaaaaaaggtgcagctcctggcttgggaatgatgactcatgtggaatttggtcttaaatttatcacatccttttgggatgtgatgattgtatcacttgttcattttgcaaagacaaggtgcactgctacaaactttggtttaatctgaaataaaacaaaactcactgagaggaagatgcatcccagtaggtgaaagtcgagaaggatttgcatgttactattacacttgctttttagtcccacatcgtctgaaacataaaatatttcagcgtttaaatacttcaagcgaaccagtaggcttg

SEQ ID NO: 78 - DNA, 102 bp, synthetic construct
  misc_feature 1..102: note = Promoter
  source 1..102: mol_type = other DNA
ataagcttatgatttcttttttcttacgaattttgcgtcccacatcggtaagcgagtgaagaaataactgctttatatatggctacaaagcaccattggtca

SEQ ID NO: 79 - DNA, 23 bp, synthetic construct
  misc_feature 1..23: note = Target site used to evaluate ABE activity
  source 1..23: mol_type = other DNA
tctcctacaaagctagagtatca

SEQ ID NO: 80 - DNA, 23 bp, synthetic construct
  misc_feature 1..23: note = Target site used to evaluate ABE activity
  source 1..23: mol_type = other DNA
tctcctacaaagctagagtaaca

SEQ ID NO: 81 - DNA, 23 bp, synthetic construct
  misc_feature 1..23: note = Target site used to evaluate ABE activity
  source 1..23: mol_type = other DNA
tctcctacaaagctagagtaagg

SEQ ID NO: 82 - DNA, 23 bp, synthetic construct
  misc_feature 1..23: note = Target site used to evaluate ABE activity
  source 1..23: mol_type = other DNA
gtcttacaagaggaaaggtgggg

SEQ ID NO: 83 - DNA, 23 bp, synthetic construct
  misc_feature 1..23: note = Target site used to evaluate ABE activity
  source 1..23: mol_type = other DNA
aagataaatatgagtcagaccgt

SEQ ID NO: 84 - DNA, 23 bp, synthetic construct
  misc_feature 1..23: note = Target site used to evaluate ABE activity
  source 1..23: mol_type = other DNA
aagataaatacgagtcagacggt

SEQ ID NO: 85 - DNA, 23 bp, synthetic construct
  misc_feature 1..23: note = Target site used to evaluate ABE activity
  source 1..23: mol_type = other DNA
aagataaatatgagtcagacggt

SEQ ID NO: 86 - DNA, 23 bp, synthetic construct
  misc_feature 1..23: note = Target site used to evaluate ABE activity
  source 1..23: mol_type = other DNA
tctccctaatattgctcgtcttt

SEQ ID NO: 87 - DNA, 23 bp, synthetic construct
  misc_feature 1..23: note = Target site used to evaluate ABE activity
  source 1..23: mol_type = other DNA
acacgggaaattacagcaggaga

SEQ ID NO: 88 - DNA, 23 bp, synthetic construct
  misc_feature 1..23: note = Target site used to evaluate ABE activity
  source 1..23: mol_type = other DNA
ggaaggcgcagatcgagtccgcg

SEQ ID NO: 89 - DNA, 23 bp, synthetic construct
  misc_feature 1..23: note = Target site used to evaluate ABE activity
  source 1..23: mol_type = other DNA
tcgtacgtacgtaccatgcatgc

SEQ ID NO: 90 - DNA, 23 bp, synthetic construct
  misc_feature 1..23: note = Target site used to evaluate ABE activity
  source 1..23: mol_type = other DNA
catagcactagcacctgcttttg

SEQ ID NO: 91 - DNA, 18 bp, synthetic construct
  misc_feature 1..18: note = Forward primer
  source 1..18: mol_type = other DNA
acgacactaaagatgtgc

SEQ ID NO: 92 - DNA, 20 bp, synthetic construct
  misc_feature 1..20: note = Reverse primer
  source 1..20: mol_type = other DNA
gggtagaaagaatggtaagt

SEQ ID NO: 93 - DNA, 22 bp, synthetic construct
  misc_feature 1..22: note = Reference probe
  source 1..22: mol_type = other DNA
acatgcctctcccttccagtag

SEQ ID NO: 94 - DNA, 20 bp, synthetic construct
  misc_feature 1..20: note = BE probe
  source 1..20: mol_type = other DNA
ctctcctacaaagctagagt

SEQ ID NO: 95 - DNA, 17 bp, synthetic construct
  misc_feature 1..17: note = Forward primer
  source 1..17: mol_type = other DNA
tccactgaccaaccaag

SEQ ID NO: 96 - DNA, 21 bp, synthetic construct
  misc_feature 1..21: note = Reverse primer
  source 1..21: mol_type = other DNA
ggtagactgaaagtaccaaga

SEQ ID NO: 97 - DNA, 19 bp, synthetic construct
  misc_feature 1..19: note = Reference probe
  source 1..19: mol_type = other DNA
tgcaaggtacagctgcagc

SEQ ID NO: 98 - DNA, 22 bp, synthetic construct
  misc_feature 1..22: note = BE probe
  source 1..22: mol_type = other DNA
agataaatatgagtcagaccgt

SEQ ID NO: 99 - DNA, 27 bp, synthetic construct
  misc_feature 1..27: note = GFP Stop upper strand
  source 1..27: mol_type = other DNA
tttgctgaagcactacacgccgtaggt

SEQ ID NO: 100 - DNA, 27 bp, synthetic construct
  misc_feature 1..27: note = GFP Stop lower strand
  source 1..27: mol_type = other DNA
acctacggcgtgtagtgcttcagcaaa

SEQ ID NO: 101 - DNA, 27 bp, synthetic construct
  misc_feature 1..27: note = GFP Gln upper strand
  source 1..27: mol_type = other DNA
tttgctgaagcactgcacgccgtaggt

SEQ ID NO: 102 - DNA, 27 bp, synthetic construct
  misc_feature 1..27: note = GFP Gln lower strand
  source 1..27: mol_type = other DNA
acctacggcgtgcagtgcttcagcaaa

SEQ ID NO: 103 - DNA, 29703 bp, synthetic construct
  source 1..29703: mol_type = other DNA
gggccctcttagaggatccccgggtaccgagctcggatatctactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtctacaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccgcacgtgtcagtaccgtctgtagatgacaacaacccgtcgtccacagtcatgtccaaaatatcctttcttcttttttttcgattcggatatgtatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggacccaccgagccccgcaagggtgctgacatcctcgtcgagtccctcgagcgctgcggcgtccgcgacgtctttgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagactcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaagtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaaacttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcctcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaagatatccaattcggcgttaattcagtacattaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagagcttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagagggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctccattactcgcatccattctcaggctgtctcgtctcgtctcctcgagatacctgatgtgcctgctatgcttaaatacatacctcgcccagtagctttccaatatgggaacatcaattgtacatcgggccgggataatcatgtcgtcacggaacttactgtaagagtaataattcaaaagagatgtcggtttgctagttcacgtaaaggtgcctcgcgccacctctaagtaagtgagccgtcgagacattatccctgattttctcactactattagtactcacggcgcaataccaccacagccttgtctcgccagaatgccggtcagcatatggaagacctcaaggcaggtcaaataacttcgtatagcatacattatacgaagttatgtattctagagctgggagttcgtagacggaaacaaacgcagaatccaagcctcgagatacctgcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgttttttctataaaaaaagggaaactgcccctcatttacagcaaattgtccgctgcctgtcgtccagatacaatgaacgtacctagtaggaactcttttacacgctcggtcgctcgccgcggatcggagtcccaggaacacgacaccactgtgtaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgctacctccgctttagttctgcatacagtcaacctaacacacccgagcatatcacagtgacagacacggctatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagagcactgaaggtcctcaatcgcactggaaacatcaaggtcgctcgagatacctaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgatcgatcgcgtcactcacgggtagctcatggtcgagcgtagcatgcaggaacttatttgccgtgcgctcccagctctccgctcgcgtgccttccagtctgtctcacactagctgctgtgggacgatcgaagtgggtgtgtcagctagctagctgcgccgtgaccacgcacatgaccgcagtgcgcgcggggctgatcaagggaaagtgatccggctatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgatcaggataactagctgagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgtattctagactgacctcctgccagcaatagtaagacaacacgcaaagtcctcgagatacctccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgatgactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagagctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacggctatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaagtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagaccaggatacatagattaccacaactccgagcccttccaccctcgagatacctgctgagctaagacggcgtcggtacatagcggtcttacggtcagtcgtaattcctcacgagtcccgtccagttgagcgtatcactcccaatgtacaagcaacccgagaaggctgtgcttggagtcaatcggatgtaggatgatctccagacacggggccaccactcttcacgcgtaaagcaagaacgtcgagcagtcatgaaagtcttagtaccggacgtgccgttttactgcgaatattacctgaagctgtaccgttattggggggcaaagatggagtcctcctcttatcatatttgtatataacttcgtatagcatacattatacgaagttatgtattctagactcgttcgctgccacctaagaatactctacggtcacatacctcgagataccttaagctaggtagtaactagtgcgactgaagcttatcgcgtatgcgagtgactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcaggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatacacgggaaattacagcaggagataatttctactaagtgtagatttttttcattactcgcatccattctccatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatggaaggcgcagatcgagtccgcgtaatttctactaagtgtagatttttttgctgggagttcgtagacggacatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagattcgtacgtacgtaccatgcatgctaatttctactaagtgtagatttttttgcactgaaggtcctcaatcgcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatcatagcactagcacctgcttttgtaatttctactaagtgtagatttttttgtatctagaatcgcgtatgcgagtgtatctagaatcgcgtatgcgagtgtattctagacaagacgctggctctgacatttccgctactgaactactcgctcgagataccttgaatggccggaagtggtacggacgcaatatgcgcgggtgagagggcaaataggcaggttcgcctacgttacgctagggggcgattctataagaatgcacattgcatcgatacataagatgtctcgaccgcatgcgcaacttgtgaagtgtctactatccctaagcccatttctcgcacaataacccctgaatgtgtccgcatctgatgttaccggggttgagttagtgtcgagctcgcggaacttattgcatgagtagagatatgtaagagctgttagatagctcgctgagctaatagtgtattctagagttccttatcatctggcgaatcggacccacaagagcactg

SEQ ID NO: 104 - DNA, 29703 bp, synthetic construct
  source 1..29703: mol_type = other DNA
gggccctcttagaggatccccgggtaccgagctcggatatctactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtctacaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccgcacgtgtcagtaccgtctgtagatgacaacaacccgtcgtccacagtcatgtccaaaatatcctttcttcttttttttcgattcggatatgtatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggacccaccgagccccgcaagggtgctgacatcctcgtcgagtccctcgagcgctgcggcgtccgcgacgtctttgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagactcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaagtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaaacttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcctcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaagatatccaattcggcgttaattcagtacattaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagagcttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagagggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctccattactcgcatccattctcaggctgtctcgtctcgtctcctcgagatacctgatgtgcctgctatgcttaaatacatacctcgcccagtagctttccaatatgggaacatcaattgtacatcgggccgggataatcatgtcgtcacggaacttactgtaagagtaataattcaaaagagatgtcggtttgctagttcacgtaaaggtgcctcgcgccacctctaagtaagtgagccgtcgagacattatccctgattttctcactactattagtactcacggcgcaataccaccacagccttgtctcgccagaatgccggtcagcatatggaagacctcaaggcaggtcaaataacttcgtatagcatacattatacgaagttatgtattctagagctgggagttcgtagacggaaacaaacgcagaatccaagcctcgagatacctgcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgttttttctataaaaaaagggaaactgcccctcatttacagcaaattgtccgctgcctgtcgtccagatacaatgaacgtacctagtaggaactcttttacacgctcggtcgctcgccgcggatcggagtcccaggaacacgacaccactgtgtaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgctacctccgctttagttctgcatacagtcaacctaacacacccgagcatatcacagtgacagacacggctatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagagcactgaaggtcctcaatcgcactggaaacatcaaggtcgctcgagatacctaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgatcgatcgcgtcactcacgggtagctcatggtcgagcgtagcatgcaggaacttatttgccgtgcgctcccagctctccgctcgcgtgccttccagtctgtctcacactagctgctgtgggacgatcgaagtgggtgtgtcagctagctagctgcgccgtgaccacgcacatgaccgcagtgcgcgcggggctgatcaagggaaagtgatccggctatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgatcaggataactagctgagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgtattctagactgacctcctgccagcaatagtaagacaacacgcaaagtcctcgagatacctccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgatgactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagagctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacggctatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaagtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagaccaggatacatagattaccacaactccgagcccttccaccctcgagatacctgctgagctaagacggcgtcggtacatagcggtcttacggtcagtcgtaattcctcacgagtcccgtccagttgagcgtatcactcccaatgtacaagcaacccgagaaggctgtgcttggagtcaatcggatgtaggatgatctccagacacggggccaccactcttcacgcgtaaagcaagaacgtcgagcagtcatgaaagtcttagtaccggacgtgccgttttactgcgaatattacctgaagctgtaccgttattggggggcaaagatggagtcctcctcttatcatatttgtatataacttcgtatagcatacattatacgaagttatgtattctagactcgttcgctgccacctaagaatactctacggtcacatacctcgagataccttaagctaggtagtaactagtgcgactgaagcttatcgcgtatgcgagtgactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtactcgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacgggtgttcaatgctcagaagaaggcccagagctccatcaactccggaggatctagcggaggctcctctggctctgagacacctggcacaagcgagagcgcaacacctgaaagcagcgggggcagcagcggggggtcaggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatacacgggaaattacagcaggagataatttctactaagtgtagatttttttcattactcgcatccattctccatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatggaaggcgcagatcgagtccgcgtaatttctactaagtgtagatttttttgctgggagttcgtagacggacatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagattcgtacgtacgtaccatgcatgctaatttctactaagtgtagatttttttgcactgaaggtcctcaatcgcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatcatagcactagcacctgcttttgtaatttctactaagtgtagatttttttgtatctagaatcgcgtatgcgagtgtatctagaatcgcgtatgcgagtgtattctagacaagacgctggctctgacatttccgctactgaactactcgctcgagataccttgaatggccggaagtggtacggacgcaatatgcgcgggtgagagggcaaataggcaggttcgcctacgttacgctagggggcgattctataagaatgcacattgcatcgatacataagatgtctcgaccgcatgcgcaacttgtgaagtgtctactatccctaagcccatttctcgcacaataacccctgaatgtgtccgcatctgatgttaccggggttgagttagtgtcgagctcgcggaacttattgcatgagtagagatatgtaagagctgttagatagctcgctgagctaatagtgtattctagagttccttatcatctggcgaatcggacccacaagagcactg

SEQ ID NO: 105 - DNA, 29697 bp, synthetic construct
  source 1..29697: mol_type = other DNA
gggccctcttagaggatccccgggtaccgagctcggatatctactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtctacaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccgcacgtgtcagtaccgtctgtagatgacaacaacccgtcgtccacagtcatgtccaaaatatcctttcttcttttttttcgattcggatatgtatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggacccaccgagccccgcaagggtgctgacatcctcgtcgagtccctcgagcgctgcggcgtccgcgacgtctttgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagactcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaagtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaaacttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcctcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaagatatccaattcggcgttaattcagtacattaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagagcttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagagggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctccattactcgcatccattctcaggctgtctcgtctcgtctcctcgagatacctgatgtgcctgctatgcttaaatacatacctcgcccagtagctttccaatatgggaacatcaattgtacatcgggccgggataatcatgtcgtcacggaacttactgtaagagtaataattcaaaagagatgtcggtttgctagttcacgtaaaggtgcctcgcgccacctctaagtaagtgagccgtcgagacattatccctgattttctcactactattagtactcacggcgcaataccaccacagccttgtctcgccagaatgccggtcagcatatggaagacctcaaggcaggtcaaataacttcgtatagcatacattatacgaagttatgtattctagagctgggagttcgtagacggaaacaaacgcagaatccaagcctcgagatacctgcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgttttttctataaaaaaagggaaactgcccctcatttacagcaaattgtccgctgcctgtcgtccagatacaatgaacgtacctagtaggaactcttttacacgctcggtcgctcgccgcggatcggagtcccaggaacacgacaccactgtgtaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgctacctccgctttagttctgcatacagtcaacctaacacacccgagcatatcacagtgacagacacggctatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagagcactgaaggtcctcaatcgcactggaaacatcaaggtcgctcgagatacctaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgatcgatcgcgtcactcacgggtagctcatggtcgagcgtagcatgcaggaacttatttgccgtgcgctcccagctctccgctcgcgtgccttccagtctgtctcacactagctgctgtgggacgatcgaagtgggtgtgtcagctagctagctgcgccgtgaccacgcacatgaccgcagtgcgcgcggggctgatcaagggaaagtgatccggctatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgatcaggataactagctgagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgtattctagactgacctcctgccagcaatagtaagacaacacgcaaagtcctcgagatacctccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgatgactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagagctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacggctatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaagtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaatcagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgagtattctagaccaggatacatagattaccacaactccgagcccttccaccctcgagatacctgctgagctaagacggcgtcggtacatagcggtcttacggtcagtcgtaattcctcacgagtcccgtccagttgagcgtatcactcccaatgtacaagcaacccgagaaggctgtgcttggagtcaatcggatgtaggatgatctccagacacggggccaccactcttcacgcgtaaagcaagaacgtcgagcagtcatgaaagtcttagtaccggacgtgccgttttactgcgaatattacctgaagctgtaccgttattggggggcaaagatggagtcctcctcttatcatatttgtatataacttcgtatagcatacattatacgaagttatgtattctagactcgttcgctgccacctaagaatactctacggtcacatacctcgagataccttaagctaggtagtaactagtgcgactgaagcttatcgcgtatgcgagtgactataagctaggtagtaactagtacctgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaacaatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtactcgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacgggtgttcaatgctcagaagaaggcccagagctccatcaacggtggcggaggttctggcggtggaggatccggaggtggcgggtccggtggtggcggatcaggtggaggaggttcgggaggcggaggtagtggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgactgcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtactcgacggccgagtactataagctaggtagtaactagtacctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatacacgggaaattacagcaggagataatttctactaagtgtagatttttttcattactcgcatccattctccatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatggaaggcgcagatcgagtccgcgtaatttctactaagtgtagatttttttgctgggagttcgtagacggacatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagattcgtacgtacgtaccatgcatgctaatttctactaagtgtagatttttttgcactgaaggtcctcaatcgcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagcaccagtggtctagtggtagaatagtaccctgccacggtacagacccgggttcgattcccggctggaaataatttctactaagtgtagatcatagcactagcacctgcttttgtaatttctactaagtgtagatttttttgtatctagaatcgcgtatgcgagtgtatctagaatcgcgtatgcgagtgtattctagacaagacgctggctctgacatttccgctactgaactactcgctcgagataccttgaatggccggaagtggtacggacgcaatatgcgcgggtgagagggcaaataggcaggttcgcctacgttacgctagggggcgattctataagaatgcacattgcatcgatacataagatgtctcgaccgcatgcgcaacttgtgaagtgtctactatccctaagcccatttctcgcacaataacccctgaatgtgtccgcatctgatgttaccggggttgagttagtgtcgagctcgcggaacttattgcatgagtagagatatgtaagagctgttagatagctcgctgagctaatagtgtattctagagttccttatcatctggcgaatcggacccacaagagcactg

SEQ ID NO: 106 - DNA, 21 bp, synthetic construct
  source 1..21: mol_type = other DNA
ggaaagttcctgcccaagaga

SEQ ID NO: 107 - DNA, 18 bp, synthetic construct
  source 1..18: mol_type = other DNA
tggtctcccatccaggcg

SEQ ID NO: 108 - DNA, 20 bp, synthetic construct
  source 1..20: mol_type = other DNA
tgcgcatcgactcgaagatc

SEQ ID NO: 109 - DNA, 19 bp, synthetic construct
  source 1..19: mol_type = other DNA
ccaatgtcgtggtctccgg

SEQ ID NO: 110 - DNA, 19 bp, synthetic construct
  source 1..19: mol_type = other DNA
agcccgaactcccaaacac

SEQ ID NO: 111 - DNA, 20 bp, synthetic construct
  source 1..20: mol_type = other DNA
gtgaggaggtggaagacgtc

SEQ ID NO: 112 - DNA, 20 bp, synthetic construct
  source 1..20: mol_type = other DNA
agagagggagttgcagttgc

SEQ ID NO: 113 - DNA, 19 bp, synthetic construct
  source 1..19: mol_type = other DNA
ggatgggccctgaagcaat

SEQ ID NO: 114 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 115 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 116 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 117 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSIN

SEQ ID NO: 118 - DNA, 5491 bp, synthetic construct
  source 1..5491: mol_type = other DNA
ttgcgatcgctctagagtatacgcgttaacgcggccgctgtacagggcccgggttaattaagcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacctgagccgaggtgaaattcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccaattgcctgcaggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagc

SEQ ID NO: 119 - DNA, 8720 bp, synthetic construct
  source 1..8720: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgctacgtacctgcaggcccgggttaattaagcggccgcaggcctaactggcccttaagggtaccgatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatcaggtgttagtttctagtttgtgcgatcgaatttgtcgattaatctgagtttttctgattaacaggtttaaacaaaaccatgaaaagaaccgccgacggcagcgaattcgagcctaagaagaagaggaaggtttccggcggcagctcaggaggttcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtactcgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacgggtgttcaatgctcagaagaaggcccagagctccatcaactccggaggatctagcggaggctcctctggctctgagacacctggcacaagcgagagcgcaacacctgaaagcagcgggggcagcagcggggggtcaggctccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttccgcaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctacccaaggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgcccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcactcaggtaaaagaaccgccgacggcagcgaattcgagcctaagaagaagaggaaggtttgattgctagcacgcgtccctagacttgtacatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 120 - DNA, 3228 bp, synthetic construct
  source 1..3228: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatttaattaattcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaatttctactaagtgtagatacctcggctcaggtcttgtagtttaatttctactaagtgtagatttttttttggcgcgccgatgagaccgaggtcgtgatgtctgaagtacgccaatacatctatggcatcggtacgcagtcctaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 121 - DNA, 3228 bp, synthetic construct
  source 1..3228: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatttaattaattcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaatttctactaagtgtagattcctacctcatctgggacatcattaatttctactaagtgtagatttttttttggcgcgccgatgagaccgaggtcgtgatgtctgaagtacgccaatacatctatggcatcggtacgcagtcctaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 122 - DNA, 3229 bp, synthetic construct
  source 1..3229: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatttaattaattcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaatttctactaagtgtagataaagtgccaccacttgggatcatctaatttctactaagtgtagatttttttttggcgcgccgatgagaccgaggtcgtgatgtctgaagtacgccaatacatctatggcatcggtacgcagtcctaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 123 - DNA, 3106 bp, synthetic construct
  source 1..3106: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatttaattaaaataaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggattgccagtggtctagtggtagaatagtaccctgccacggtacagacccgggttcgattcccggctggaaataatttctactaagtgtagatgtcccttatttctcatggaaaattaatttctactaagtgtagatttttttttggcgcgccgatgagaccgaggtcgtgatgtctgaagtacgccaatacatctatggcatcggtacgcagtcctaggatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca